(19) United States
(12) Patent Application Publication
(10) Pub. No.: US 2026/0209773 A1
(43) Pub. Date: Jul. 23, 2026

(54) COMPOSITIONS AND METHODS FOR TUNABLE REGULATION OF RNAi

(71) Applicant: OBSIDIAN THERAPEUTICS, INC., Cambridge, MA (US)

(72) Inventors: Jan ter Meulen, Cambridge, MA (US); Dhruv Sethi, Westwood, MA (US); Mara Christine Inniss, Beverly, MA (US)

(73) Assignee: OBSIDIAN THERAPEUTICS, INC., Cambridge, MA (US)

(21) Appl. No.: 19/453,178

(22) Filed: Jan. 20, 2026

Related U.S. Application Data
(60) Provisional application No. 63/747,704, filed on Jan. 21, 2025.
(60) Provisional application No. 63/839,528, filed on Jul. 7, 2025.

(51) Int. Cl. C12N 15/113; A61P 37/06; C12N 15/85

(52) U.S. Cl. CPC C12N 15/1137; A61P 37/06; C12N 15/1136; C12N 15/85; C12N 2310/141; C12N 2310/531; C12N 2830/002; C12N 2830/34; C12Y 105/01003; C12Y 402/01001

(57) ABSTRACT

The present disclosure provides compositions and methods related to drug responsive domain (DRD)-regulatable RNA interference (RNAi). Such compositions and methods provide for inhibitory RNA molecule expression driven by regulated transcriptional activity.

### CROSS REFERENCE TO RELATED APPLICATION

This application claims priority to U.S. Provisional Application No. 63/747,704, filed Jan. 21, 2025, and U.S. Provisional Application No. 63/839,528, filed Jul. 7, 2025, both of which are incorporated by reference herein in their entirety.

### SEQUENCE LISTING

The instant application contains a Sequence Listing that has been filed electronically in .xml format and is hereby incorporated by reference in its entirety. Said .xml copy, created on Jan. 18, 2026, is named “108407-1540226-059US1.xml” and is 115,551 bytes in size.

### BACKGROUND

RNA interference (RNAi) is a naturally occurring mechanism that involves sequence-specific down regulation of messenger RNA (mRNA) to reduce the amount of protein that is expressed. Methods for inducing RNA interference involve, for example, the use of short hairpin RNA (shRNA) to target a gene of choice. RNAi gene therapy approaches have been proposed as treatment for various disorders, however, current technologies do not allow titration of the timing or levels of target gene regulation. This has rendered many potential RNAi therapy applications difficult or impossible to safely and effectively deploy. A need exists for regulation of the RNAi for optimization of therapeutic benefits and to facilitate its widespread adoption.

### BRIEF SUMMARY

Provided herein are sets of nucleic acid sequences comprising a first nucleic acid sequence that encodes a transcription factor activation domain, a second nucleic acid sequence that encodes a transcription factor DNA binding domain that binds to a specific nucleotide binding site, a third nucleic acid sequence that encodes a drug responsive domain (DRD), wherein at least one of the transcription factor activation domain, the transcription factor DNA binding domain, or the combination of the transcription factor activation domain and the transcription factor DNA binding domain is operably linked to the DRD, and a fourth nucleic acid sequence that encodes an inhibitory RNA molecule, the fourth nucleic acid sequence being operably linked to an inducible promoter comprising the specific nucleotide binding site, wherein the transcription factor activation domain and the transcription factor DNA binding domain interact to form a transcription factor capable of activating transcription of the inhibitory RNA molecule upon binding to the specific nucleotide binding site. The set of nucleic acid sequences can be carried in a single vector or in separate vectors, in various configurations.

Also provided herein are engineered cells comprising the set of nucleic acid sequences, and pharmaceutical compositions comprising the engineered cells. Also provided are methods of producing an engineered cell comprising introducing into the cell the set of nucleic acid sequences. Also provided are methods of delivering a regulatable inhibitory RNA molecule to a subject and methods for treating a disease in a subject in need thereof comprising administering the set of nucleic acids, engineered cells, or pharmaceutical compositions to the subject. Also provided are methods for regulation of an immune cell comprising administering to the immune cell the set of nucleic acid sequences. A kit comprising the set of nucleic acid sequences, the engineered cells, or the pharmaceutical compositions, and a ligand, are also provided.

### DESCRIPTION OF THE DRAWINGS

The present application includes the following figures intended to illustrate certain embodiments and/or features of the methods and compositions herein, and to supplement any description(s) of the methods and compositions. The figures do not limit the scope of the methods and compositions herein.

FIG. 1 is a schematic showing an exemplary construct comprising the set of nucleic acid sequences described herein.

FIG. 2 is an exemplary graph of ligand (drug)-regulated shRNA expression, and consequent target protein or RNA expression, according to the compositions and methods provided herein.

FIG. 3A are graphs showing GFP expression in HEK293 T cells transduced with constructs TXN-041 and TXN-051 delivered in dual vectors.

FIG. 3B are graphs showing mCherry expression, and thus shRNA expression, in HEK293 T cells transduced with constructs TXN-041 and TXN-051 delivered in dual vectors.

FIG. 4 is a western blot showing apolipoprotein E (apoE) expression compared an actin control in HepG2 cells transduced with constructs TXN-041 and ApoE_Ri-004 delivered in dual vectors.

FIG. 5 are graphs showing apoE and mCherry expression in in HepG2 cells transduced with constructs TXN-041 and ApoE_Ri-004 delivered in dual vectors.

FIG. 6 is a schematic showing the experimental design for western blot analysis of Huntingtin (HTT) expression in U87 cells transduced with constructs TXN-041 and TXN-051 delivered in dual vectors.

FIG. 7 is a western blot showing HTT expression compared to an actin control in in U87 cells transduced with constructs TXN-041 and TXN-051 delivered in dual vectors.

FIG. 8 is a flow cytometry plot showing GFP expression in HEK293 T cells transduced with EGFP-038 and single vector construct TXN-059.

FIG. 9A is a graph showing GFP expression as a function of cell count in HEK293 T cells transduced with EGFP-038 and single vector construct TXN-059.

FIG. 9B is a graph showing GFP expression with various virus volumes in HEK293 T cells transduced with EGFP-038 and single vector construct TXN-059.

FIG. 10A is a graph showing mCherry, and thus shRNA, expression as a function of cell count in HEK293 T cells transduced with EGFP-038 and single vector construct TXN-059.

FIG. 10B is a graph showing mCherry, and thus shRNA, expression with various virus volumes in HEK293 T cells transduced with EGFP-038 and single vector construct TXN-059.

FIG. 11A are flow cytometry plots showing mCherry, and thus shRNA, expression in Jurkat T cells transduced with constructs TXN-077, -078, and -079 delivered in a single vector.

FIG. 11B is a graph showing NFAT-luciferase, and thus ZAP70 signaling activity in Jurkat T cells transduced with constructs TXN-077, -078, and -079 delivered in a single vector.

FIG. 12 is a graph showing NFAT-luciferase, and thus LAT signaling activity in Jurkat T cells transduced with 10 or 30 μl constructs TXN-080, -081, and -082 delivered in a single vector.

FIG. 13 is a graph showing NFAT-luciferase, and thus Lck signaling activity in Jurkat T cells transduced with 10 or 30 μl constructs TXN-123, -124, and -125 delivered in a single vector.

FIG. 14A is a graph showing caspase 3/7 activity, and thus Nalm6 target cell killing, in CD19 CAR T cells transduced with TXN-081 at a 3:1 effector:target (E:T) cell ratio.

FIG. 14B is a graph showing caspase 3/7 activity, and thus Nalm6 target cell killing, in CD19 CAR T cells transduced with TXN-082 at a 3:1 effector:target (E:T) cell ratio.

FIG. 14C is a graph showing caspase 3/7 activity, and thus Nalm6 target cell killing, in CD19 CAR T cells transduced with TXN-080 at a 3:1 effector:target (E:T) cell ratio.

FIG. 15A is a graph showing caspase 3/7 activity, and thus Nalm6 target cell killing, in CD19 CAR T cells transduced with TXN-078 at a 10:1 effector:target (E:T) cell ratio.

FIG. 15B is a graph showing caspase 3/7 activity, and thus Nalm6 target cell killing, in CD19 CAR T cells transduced with TXN-078 at a 10:1 effector:target (E:T) cell ratio.

FIG. 15C is a graph showing caspase 3/7 activity, and thus Nalm6 target cell killing, in CD19 CAR T cells transduced with TXN-124 at a 10:1 effector:target (E:T) cell ratio.

FIG. 15D is a graph showing caspase 3/7 activity, and thus Nalm6 target cell killing, in CD19 CAR T cells transduced with TXN-076 at a 10:1 effector:target (E:T) cell ratio.

FIG. 16A is a graph showing mCherry, and thus shRNA, expression in T cells transduced with TXN-078.

FIG. 16B is a graph showing mCherry, and thus shRNA, expression in T cells transduced with TXN-080.

FIG. 16C is a graph showing mCherry, and thus shRNA, expression in T cells transduced with TXN-081.

FIG. 16D is a graph showing mCherry, and thus shRNA, expression in T cells transduced with TXN-082.

### DETAILED DESCRIPTION

The following description recites various aspects and embodiments of the present compositions and methods. No particular embodiment is intended to define the scope of the compositions and methods. Rather, the embodiments merely provide non-limiting examples that are at least included within the scope of the disclosed compositions and methods.

The present disclosure provides compositions (e.g., sets of nucleic acid sequences and cells), methods, and kits for regulating RNA interference (RNAi) via transcriptional regulation of inhibitory RNA molecules.

### Nucleic Acids

Provided herein is a set of nucleic acid sequences comprising a first nucleic acid sequence that encodes a transcription factor activation domain; a second nucleic acid sequence that encodes a transcription factor DNA binding domain that binds to a specific nucleotide binding site; a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and a fourth nucleic acid sequence that encodes an inhibitory RNA molecule, the fourth nucleic acid sequence being operably linked to an inducible promoter comprising the specific nucleotide binding site; wherein the transcription factor activation domain and the transcription factor DNA binding domain interact to form a transcription factor capable of activating transcription of the inhibitory RNA molecule upon binding to the specific nucleotide binding site.

As used throughout, the term nucleic acid or polynucleotide refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar properties as the reference nucleic acid. A nucleic acid sequence can comprise combinations of deoxyribonucleic acids and ribonucleic acids. Such deoxyribonucleic acids and ribonucleic acids include both naturally occurring molecules and synthetic analogues. Nucleic acids also encompass all forms of sequences including, but not limited to, single-stranded forms, double-stranded forms, hairpins, stem-and-loop structures, and the like.

As used herein, a transcription factor is a protein that binds to DNA, preferably to a sequence-specific site on the DNA (i.e., a specific nucleotide binding site) located in or near a promoter, which facilitates the binding of the transcription machinery to the promoter, thus activating transcription of the DNA sequence. Optionally, a transcription factor for use in the compositions and methods described herein comprises a transcription factor DNA binding domain and a transcription factor activation domain which combine to result in a functional transcription factor.

Optionally, the transcription factor activation domain described herein is selected from the group consisting of p65, VP16, NFAT1, VP64, p300, synergistic activation mediator (SAM), VPR. Optionally, the transcription factor activation domain is p65. Optionally, the transcription factor activation domain comprises SEQ ID NO: 37.

The transcription factor DNA binding domain may be derived from an existing nucleic acid binding protein. For example, a DNA-binding sequence or domain of an existing DNA binding protein may be used as or further modified to generate the transcription factor DNA binding domain of the present disclosure. Optionally, the transcription factor DNA binding domain described herein is selected from the group consisting of a zinc finger protein, Cas9, Cas12, and TAL. Optionally, the transcription factor DNA binding domain is an engineered zinc finger protein. Optionally, the transcription factor DNA binding domain is zinc finger homeodomain 1 (ZFHD1). ZFHD1 is a zinc finger-homeodomain fusion protein designed by Pomerantz, J. L., et al. (see Pomerantz, J. L., et al., Science, 267:5194, (1995)). ZFHD1 comprises fingers 1 and 2 of Zif268, a gly-gly-arg-arg linker, and the OCT-1 homeodomain. ZFHD1 can bind to a nucleic acid sequence comprising the sequence TAATGATGGGCG (SEQ ID NO: 40). In some embodiments, the transcription factor DNA binding domain consists of or comprises the amino acid sequence of ZFHD1. Optionally, the transcription factor DNA binding domain comprises SEQ ID NO: 38.

The transcription factor activation domain and the transcription factor DNA binding domain may be separately selected and combined to form the transcription factor. The combination of transcription factor activation domain and transcription factor DNA binding domain may be selected by one skilled in the art based on desired characteristics, including DNA binding domains of DNA binding proteins discussed below. Optionally, the transcription factor activation domain is p65 (SEQ ID NO: 37) and the transcription factor DNA binding domain is ZFHD1 (SEQ ID NO: 38).

The recognition of DNA sequences by transcription factors occurs by chemical interactions of the amino acid side chains of a transcription factor protein with base pair residues of the DNA that functions as regulatory sequence. The DNA sequence that a transcription factor DNA binding domain binds to may be referred to as a transcription factor-binding site or response element, or as used interchangeably herein, a specific nucleotide binding site. The specific nucleotide binding site described herein may be located in or near a promoter. Optionally, the specific nucleotide binding site is located within a promoter. Optionally, the specific nucleotide binding site is located near (e.g., within 500 base pairs upstream or downstream of) a promoter. Thus, optionally, the encoded transcription factor (i.e., the transcription factor activation domain and the transcription factor DNA binding domain) binds the specific nucleotide binding site and regulates expression of a nucleic acid sequence controlled by the promoter comprising or near the specific nucleotide binding site. Pairs of transcription factors (including engineered or synthetic transcription factors comprising a transcription factor activation domain and a transcription factor DNA binding domain as described herein) and their corresponding nucleotide binding sites are known in the art. Also known are DNA binding domains of DNA binding proteins along with their corresponding nucleotide binding sites and method for identifying new DNA binding domain sequences and corresponding nucleotide binding sites that can be used for the design of synthetic transcription factors and corresponding synthetic promoters.

Optionally, the promoter comprising or near the specific nucleotide binding site is an exogenous inducible promoter. Optionally, the promoter is an RNA polymerase II promoter, meaning any promoter to which RNA polymerase II binds. Optionally, the promoter is derived from ubiquitin C (UbiC), phosphoglycerate kinase (PGK), cytomegalovirus (CMV), beta-actin with an upstream CMV IV enhancer (CAGGS), or elongation factor 1 alpha (EF1A). Optionally, the promoter is derived from ubiquitin C (UbiC), phosphoglycerate kinase (PGK), cytomegalovirus (CMV), beta-actin with an upstream CMV IV enhancer (CAGGS), or elongation factor 1 alpha (EF1A). Optionally, the promoter is a variant of ubiquitin C (UbiC), phosphoglycerate kinase (PGK), cytomegalovirus (CMV), beta-actin with an upstream CMV IV enhancer (CAGGS), or elongation factor 1 alpha (EF1A). Optionally, the promoter is a UbiC promoter, e.g., as described in Giering et al., Molecular Therapy, 2008, Lebbink et al., PLOS One, 2011, or Zhou et al., Nucleic Acids Res., 2005. Optionally, the promoter is human ubiquitin C (hUbC). Optionally, the promoter comprises SEQ ID NO: 1. Optionally, the promoter comprises SEQ ID NO: 2. Optionally, the promoter comprises SEQ ID NO: 3.

Optionally, the promoter is a minimal promoter (also referred to as a “min promoter” or “core promoter”). The term “minimal promoter” refers to a minimal structure that enables the formation of the initiation complex. A minimal promoter may comprise an RNA polymerase binding site, TATA box and transcription start site. A minimal promoter may be coupled with one or more response elements (such as enhancers or transcription factor binding sites) to generate an inducible promoter. Additional details regarding minimal promoters and coupling of minimal promoters to response elements is provided by Ede and colleagues (Ede et al., ACS Synth Biol. 2016 May 20; 5 (5): 395-404). Optionally, the inducible promoter of a transcription factor system or component thereof of the present disclosure comprises a minimal promoter selected from the following minimal promoters: minCMV, CMV53 (minCMV with the addition of an upstream GC box), minSV40 (minimal simian virus 40 promoter), miniTK (the −33 to +32 region of the Herpes simplex thymidine kinase promoter), MLP (the −38 to +6 region of the adenovirus major late promoter), pJB42CAT5 (a minimal promoter derived from the human junB gene), YB_TATA (a synthetic minimal promoter developed by Benenson and colleagues (Hansen, J. et al. Proc Natl Acad Sci USA. 2014; 111:15705-15710)), and the TATA box alone.

As described above, the specific nucleotide binding site may comprise at least one nucleic acid site with a specific sequence that is recognized and bound by the transcription factor DNA binding domain. Optionally, the specific nucleotide binding site comprises two or more nucleic acid sites, each with a specific sequence that is recognized and bound by the transcription factor DNA binding domain. Pairing of DNA binding domains with their corresponding nucleotide binding sites is discussed above.

In addition to comprising the nucleic acid sequences described herein, the set of nucleic acid sequences of the present disclosure may comprise additional nucleic acid sequences. Additional nucleic acid sequences include, but are not limited to, regulatory elements, polyadenylation sequences, linkers, and cleavage sites.

Exemplary nucleic acid constructs that may be used individually (as a single construct) or in combination as part of a transcription factor system are described in Table 1.

TABLE 1
Exemplary constructs of transcription
factor systems and components thereof.

| DESCRIPTION | SEQ ID NO: |
|---|---|
| Promoter UbiC | 1 |
| HUbC promoter short | 2 |
| UbiC promoter + miR-30-like hairpin | 3 |
| EGFP shRNA (TXN-051, -059) | 4 |
| ZAP70 shRNA (TXN-077) | 5 |
| ZAP70 shRNA (TXN-078) | 6 |
| ZAP70 shRNA (TXN-079) | 7 |
| LAT shRNA (TXN-080) | 8 |
| LAT shRNA (TXN-081) | 9 |
| LAT shRNA (TXN-082) | 10 |
| ApoE shRNA (ApoE_Ri-001) | 11 |
| ApoE shRNA (ApoE_Ri-002) | 12 |
| ApoE shRNA (ApoE_Ri-003) | 13 |
| ApoE shRNA (ApoE_Ri-004) | 14 |
| HTT shRNA (HTT-001) | 15 |
| HTT shRNA (HTT-002) | 16 |
| HTT shRNA (HTT-003) | 17 |
| TXN-036 | 18 |
| (HubC short promoter- PEST-tagged EGFP) | |
| TXN-037 | 19 |
| (UbiC promoter- PEST-tagged EGFP) | |
| TXN-038 | 20 |
| (UbiC promoter plus miR-30-like hairpin- | |
| PEST-tagged EGFP) | |
| TXN-041 | 21 |
| (ZFHD1-hDHFR) | |
| TXN-051 | 22 |
| (mCherry-shRNA targeting GFP) | |
| ApoE_Ri-004 | 23 |
| (mCherry-shRNA targeting ApoE) | |
| HTT-001 | 24 |
| (mCherry-shRNA targeting HTT) | |
| TXN-059 | 25 |
| (12xZFHD1 YB-TATA-mCherry-shRNA | |
| targeting GFP-EFS-ZFHD1-hDHFR-MND- | |
| Thy 1.2) | |
| TXN-077 | 26 |
| (12xZFHD1 YB-TATA-mCherry-shRNA | |
| targeting ZAP70 -EFS-ZFHD1-hDHFR- | |
| MND-CD19 CAR) | |
| TXN-078 | 27 |
| (12xZFHD1 YB-TATA-mCherry-shRNA | |
| targeting ZAP70-EFS-ZFHD1-hDHFR- | |
| MND-CD19 CAR) | |
| TXN-079 | 28 |
| (12xZFHD1 YB-TATA-mCherry-shRNA | |
| targeting ZAP70-EFS-ZFHD1-hDHFR- | |
| MND-CD19 CAR) | |
| TCR-004 (EF1a-TCR control C128) | 29 |
| Carbonic anhydrase II (CA2) | 30 |
| Human FKBP (FK506 binding protein) | 31 |
| E. coli dihydrofolate reductase (ecDHFR) | 32 |
| Human dihydrofolate reductase (hDHFR) | 33 |
| Human estrogen receptor (ER) | 34 |
| Phosphodiesterase 5 (PDE5), full-length | 35 |
| Phosphodiesterase 5 (PDE5), ligand binding | 36 |
| domain | |
| P65 transcription factor activation domain | 37 |
| ZFHD1 transcription factor DNA binding | 38 |
| domain | |
| EGFP-038 | 39 |
| (EF1a-EGFP-PEST) | |
| ZFHD1 binding sequence | 40 |
| LCK shRNA (TXN-123) | 41 |
| LCK shRNA (TXN-124) | 42 |
| LCK shRNA (TXN-125) | 43 |
| TXN-076 | 44 |
| (12xZHFD1-YB-TATA-mCherry-shRNA | |
| targeting EGFP-EFS-Nls-Zfhd1-ierNLS-p65- | |
| GGSGGGSG-hDHFR(134)-MND- | |
| CD19CAR) | |
| TXN-080 (12xZHFD1-YB-TATA-mCherry- | 45 |
| shRNA targeting LAT-EFS-Nls-Zfhd1- | |
| ierNLS-p65-GGSGGGSG-hDHFR(134)- | |
| MND-CD19CAR) | |
| TXN-081 (12xZHFD1-YB-TATA-mCherry- | 46 |
| shRNA targeting LAT-EFS-Nls-Zfhd1- | |
| ierNLS-p65-GGSGGGSG-hDHFR(134)- | |
| MND-CD19CAR) | |
| TXN-082 | 47 |
| (12xZHFD1-YB-TATA-mCherry-shRNA | |
| targeting LAT-EFS-Nls-Zfhd1-ierNLS-p65- | |
| GGSGGGSG-hDHFR(134)-MND- | |
| CD19CAR) | |
| TXN-123 | 48 |
| (12xZHFD1-YB-TATA-mCherry-shRNA | |
| targeting Lck-EFS-Nls-Zfhd1-ierNLS-p65- | |
| GGSGGGSG-hDHFR(134)-MND- | |
| CD19CAR) | |
| TXN-124 | 49 |
| (12xZHFD1-YB-TATA-mCherry-shRNA | |
| targeting Lck-EFS-Nls-Zfhd1-ierNLS-p65- | |
| GGSGGGSG-hDHFR(134)-MND- | |
| CD19CAR) | |
| TXN-125 | 50 |
| (12xZHFD1-YB-TATA-mCherry-shRNA | |
| targeting Lck-EFS-Nls-Zfhd1-ierNLS-p65- | |
| GGSGGGSG-hDHFR(134)-MND- | |
| CD19CAR) | |

Optionally, the transcription factor activation domain and transcription factor DNA binding domain described herein interact to regulate transcription of an inhibitory RNA molecule operably linked to an inducible promoter comprising the specific nucleotide binding site.

As used herein, “inhibitory RNA” or “inhibitory RNA molecule” refers to an RNA molecule which inhibits or decreases the mRNA expression level and/or the activity of a target gene. An inhibitory RNA molecule may inhibit or decrease transcription of the gene, for example, by binding to a specific nucleotide binding site on the gene and/or inhibiting interaction between the gene and another protein or nucleic acid. Optionally, the inhibitory RNA molecule decreases mRNA expression for a target gene at least about 5%, about 10%, about 20%, about 30%, about 40%, about 50%, about 60%, about 70%, about 80%, about 90%, about 95%, about 99%, about 100% of the mRNA level found in the cell without the presence of the inhibitory RNA molecule. In one preferred embodiment, the mRNA levels are decreased by at least about 70%, about 80%, about 90%, about 95%, about 99%, about 100%. Optionally, the inhibitory RNA molecule is a small interfering RNA (siRNA), an antisense RNA, microRNA (miRNA), a CRISPR RNA, or short hairpin RNA (shRNA) molecule.

Optionally, the inhibitory RNA molecule is a shRNA molecule. As used herein, “shRNA” or an “shRNA molecule” refers to an artificial RNA molecule with a hairpin turn that can be used to silence target gene expression via the small interfering RNA (siRNA) it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors are discussed in detail below, and include, but are not limited to, adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA molecule is exported from the nucleus, then processed by Dicer and loaded into the RNA-induced silencing complex (RISC). The sense strand is degraded by RISC and the antisense strand directs RISC to an mRNA that has a complementary sequence. In the RISC, Ago2 cleaves the mRNA, or in some cases, represses translation of the mRNA, leading to its destruction and an eventual reduction in the protein encoded by the mRNA. Thus, the shRNA molecule leads to targeted gene silencing.

Optionally, the shRNA molecule is derived from a micro RNA (miRNA)-based scaffold. MicroRNAs, i.e. miRNA, are guide strands that originate from double stranded RNA molecules that are endogenously expressed e.g. in mammalian cells. A miRNA is processed from a pre-miRNA precursor molecule, similar to the processing of an shRNA, as described above, by the RNAi machinery and incorporated in an activated RNA-induced silencing complex (RISC) (See, e.g., Tijsterman & Plasterk, Cell, 2; 1 17 (1): 1-3 (2004). A pre-miRNA is a hairpin RNA molecule that can be part of a larger RNA molecule (pri-miRNA), e.g. comprised in an intron, which is first processed by Drosha to form a pre-miRNA hairpin molecule. The pre-miRNA molecule is an shRNA-like molecule that can subsequently be processed by Dicer to result in an siRNA-like double stranded RNA duplex. The miRNA, i.e. the guide strand, that is part of the double stranded RNA duplex is subsequently incorporated in RISC.

An RNA molecule such as present in nature, i.e. a pri-miRNA, a pre-miRNA or a miRNA duplex, may be used as a scaffold for producing the shRNA molecule that targets a gene of choice. Based on the predicted RNA structure of the shRNA molecule, e.g. as predicted using e.g. m-fold software using standard settings (see, e.g., Zuker, Nucleic Acids Res. 31 (13), 3406-3415, 2003), the natural miRNA sequence as it is present in the RNA structure (i.e. duplex, pre-miRNA or pri-miRNA), and the sequence present in the structure that is substantially complementary therewith are removed and replaced with a nucleic acid sequence encoding an shRNA molecule as described herein (e.g., SEQ ID NOs: 4-8) (see, e.g., Liu, Nucleic Acids Res., 36(9):281 1-24 (2008)).

Optionally, the inhibitory RNA molecule is an siRNA molecule. As discussed above, “siRNA” or an “siRNA molecule” refers to a nucleic acid that forms a double stranded RNA, which double stranded RNA has the ability to reduce or inhibit expression of a gene or target gene when the siRNA is present or expressed in the same cell as the target gene. Optionally, the inhibitory RNA molecule is an antisense RNA molecule. As used herein, “antisense RNA” or “antisense RNA molecule” refers to a single-stranded RNA that is complementary and binds to mRNA and blocks its translation into proteins. Optionally, the inhibitory RNA molecule is a micro RNA (miRNA) molecule. As used herein, “miRNA” or “miRNA molecule” refers to a class of small RNAs, some of which are known to regulate the expression of protein-coding genes at the posttranscriptional level, specifically by modulating the productive utilization of mRNA.

Optionally, the inhibitory RNA molecule targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a gene. A target sequence in the gene may be a portion of the gene comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides.

Optionally, the inhibitory RNA molecule described herein may target a gene encoding a pathogenic protein. As used herein, “pathogenic protein” refers to a protein which is, e.g., dysfunctional, overexpressed, or underexpressed, such that the protein underlies, at least in part, pathogenesis. Optionally, the inhibitory RNA molecule targets a gene encoding a pathogenic protein involved in a neurodegenerative disease, for example, Alzheimer's disease, Huntington's disease, Parkinson's disease, multiple system atrophy, amyotrophic lateral sclerosis, ataxia, Lewy body disease, frontotemporal dementia, and vascular dementia. Optionally, the inhibitory RNA molecule targets a gene encoding a pathogenic protein involved in Alzheimer's disease or Huntington's disease. Optionally, the inhibitory RNA molecule targets a gene encoding apolipoprotein E (apoE) or Huntingtin (HTT). Optionally, the inhibitory RNA molecule comprises a nucleic acid sequence of SEQ ID NOs: 5-7. Optionally, the inhibitory RNA molecule comprises a nucleic acid sequence of SEQ ID NOs: 8-10. Optionally, the inhibitory RNA molecule comprises a nucleic acid sequence of SEQ ID NOs: 11-14. Optionally, the inhibitory RNA molecule comprises a nucleic acid sequence of SEQ ID NOs: 15-17. Optionally, the inhibitory RNA molecule targets a gene encoding a pathogenic protein involved in a degenerative or inflammatory joint disease, for example osteoarthritis, rheumatoid arthritis, spondyloarthritis, fibromyalgia, systemic lupus, or gout. Optionally, the inhibitory RNA molecule targets a gene encoding a pathogenic protein involved in an ocular disease, for example age-related macular degeneration.

Optionally, the inhibitory RNA molecule targets a gene responsible for T cell receptor signaling. Optionally, the inhibitory RNA molecule targets Lck, zeta-chain-associated protein kinase-70 (ZAP70), unc119, or linker for activation of T cells (LAT). Optionally, the inhibitory RNA molecule comprises a nucleic acid sequence of SEQ ID NOs: 5-7. Optionally, the inhibitory RNA molecule comprises a nucleic acid sequence of SEQ ID NOs: 8-10. Optionally, the inhibitory RNA molecule comprises a nucleic acid sequence of SEQ ID NOs: 11-14. Optionally, the inhibitory RNA molecule comprises a nucleic acid sequence of SEQ ID NOs: 15-17.

Optionally, the transcription factor activation domain, the transcription factor DNA binding domain, and/or the combination of the transcription factor activation domain and the transcription factor DNA binding domain may be operably linked to a drug responsive domain (DRD). Such arrangement, i.e., the DRD operably linked to the transcription factor activation domain, the transcription factor DNA binding domain, or the combination of the transcription factor activation domain and the transcription factor DNA binding domain, is collectively referred to herein as DRD-TF. Without meaning to be limited by theory, DRDs are thought to be unstable polypeptides that degrade in the absence of their corresponding stabilizing ligand (also referred to as the paired ligand or ligand), but whose stability is rescued by binding to the stabilizing ligand. Because binding of the ligand to the DRD is reversible, later removal of the ligand results in the DRD unfolding, becoming unstable, and ultimately being tagged for degradation by the ubiquitin-proteasome system (“UPS”). Accordingly, it is believed that when a DRD is operably linked to a payload, like the transcription factor activation domain, the transcription factor DNA binding domain, and/or the combination of the transcription factor activation domain and the transcription factor DNA binding domain, the entire construct (i.e., the DRD and transcription factor activation domain and/or transcription factor DNA binding domain) itself is rendered unstable and degraded by the UPS. However, in the presence of the paired ligand, the construct is stabilized, and the payload remains available. Further, it is believed that the conditional nature of DRD stability allows a rapid and non-perturbing switch from stable protein to unstable UPS substrate and may facilitate regulation and/or modulation of the payload's activity level.

Payloads should be understood to include one or more polypeptides having one or more functions, such as one or more biological activities, desired to be regulated. Reference to a biological activity is understood to mean a desired activity under appropriate conditions even if not so stated. Payloads suitable for the present disclosure include transcription factor activation domains, transcription factor DNA binding domains, or a combination of the transcription factor activation domain and the transcription factor DNA binding domain.

Because the abundance and availability of a payload are related to the activity of a payload, for purposes of this disclosure, the terms abundance, availability, activity, and the phrase abundance and/or activity (and similarly level of abundance, level of availability, level of activity, and level of abundance and/or activity) are used interchangeably throughout this disclosure and are generally referred to as activity, unless explicitly stated otherwise or nonsensical in context. Further, measurements of abundance or availability are used as a proxy for activity level and may be used herein to reflect the activity level. Consequently, changes in the abundance or availability of a payload in the presence of an effective amount of ligand as compared to in the absence of ligand optionally serves as a proxy for measuring changes in activity level.

As used herein, operably linked generally means that two moieties are directly or indirectly linked such that one moiety influences the other moiety. In the context of the DRD being operably linked to at least one of the transcription factor activation domain, the transcription factor DNA binding domain, or the combination of the transcription factor activation domain and the transcription factor DNA binding domain, operable linkage means that the DRD is linked to the transcription factor activation domain and/or the transcription factor DNA binding domain directly or indirectly so as to alter a measurable characteristic of the transcription factor activation domain and/or the transcription factor DNA binding domain in the presence of the paired ligand. For example, ligand-bound DRD can alter the amount or activity of the transcription factor activation domain and/or the transcription factor DNA binding domain as compared to the level of activity in the absence of the paired ligand. Optionally, the measured amount and/or activity of the transcription factor activation domain and/or the transcription factor DNA binding domain increases in the presence of an effective amount of ligand as compared to the measured level of amount or activity in the absence of ligand.

When a DRD operably linked to at least one of the transcription factor activation domain, the transcription factor DNA binding domain, or the combination of the transcription factor activation domain and the transcription factor DNA binding domain, is exposed to a paired ligand, the DRD-TF is stabilized. The stabilized DRD-TF is then able to bind to the specific nucleotide binding site to which the transcription factor DNA binding domain binds, and thus regulate transcription of the nucleic acid sequence encoding the inhibitory RNA molecule as described herein. In the absence of the exogenous stabilizing ligand, the DRD-TF is degraded and unable to activate transcription of the inhibitory RNA molecule. Thus, both the amount and the timing of inhibitory RNA molecule expression can be controlled by administering the ligand to the cell or organism.

The DRDs, by way of example, can be chosen from carbonic anhydrase II (CA2; SEQ ID NO: 30), FK506 binding protein (FKBP; SEQ ID NO: 31), E. coli dihydrofolate reductase (ecDHFR; SEQ ID NO: 32), human dihydrofolate reductase (hDHFR; SEQ ID NO: 33), human estrogen receptor (ER; SEQ ID NO: 34), phosphodiesterase 5 (PDE5) full-length (SEQ ID NO: 35), PDE5 ligand binding domain (SEQ ID NO: 36), and or a ligand-binding portion thereof. Optionally, the DRD is hDHFR (SEQ ID NO: 33). Optionally, the DRD is CA2 (SEQ ID NO: 30). As used herein, a ligand-binding portion thereof refers to a portion of any of the foregoing DRDs that maintains DRD function, or an amino acid sequence having at least 85%, 86%, 87%, 88%, 89%, 90%, 91%, 92%, 93%, 94%, 95%, 96%, 97%, 98%, 99% or 100% identity to SEQ ID NOs: 21-27 or the DRD functional portion thereof. U.S. Pat. Nos. 9,487,787 and 10,137,180, U.S. Publication Nos.: 2019/0192691; 2020/0101142; 2020/0172879; 2021/0069248, and U.S. Pat. App. Nos.: 17,251,635; and Ser. No. 17/288,373, the contents of each of which are hereby incorporated by reference in their entirety, provide examples of DRDs (and their paired ligands) according to this disclosure. Certain of these and other exemplary DRDs suitable for use according to this disclosure are also provided elsewhere in this specification.

The DRD suitable for use in the present disclosure may comprise one or more mutations. One or more mutations (including truncations, substitutions, and deletions or any combination thereof) in the amino acid sequence of CA2, FKBP, ecDHFR, hDHFR, ER, and PDE5, for example, can be advantageous to further destabilize the DRD in the absence of the ligand. Optionally, the DRD is derived from hDHFR and comprises Y122I mutation relative to SEQ ID NO: 33. Optionally, the hDHFR mutant further comprises at least one mutation selected from the group consisting of: Q36E, Q36S, Q36T, Q36H, Q36R, K55R, N65K, N65L, N65R, N65H, N65W, Q103E, Q103S, Q103H, N108D, E162G, and K174N relative to SEQ ID NO: 33. Optionally, a DRD of the present disclosure may be derived from CA2 and comprise amino acids 2-260 of the parent CA2 sequence (SEQ ID NO: 30). This is referred to herein as a CA2 M1 deletion (M1del) mutation. Optionally, a DRD of the present disclosure comprises a region of or the whole human carbonic anhydrase 2, and further comprises one or more mutations selected from Mldel, L156H, and S56N relative to SEQ ID NO: 30.

Examples of stabilizing ligands and their uses for specific DRDs described herein are shown in Table 2 and are described in U.S. Pat. No. 9,487,787, filed Mar. 33, 2012; U.S. Pat. No. 10,137,180, filed Sep. 6, 2013; PCT Application No. PCT/US2018/037005, filed Jun. 12, 2018; PCT Application No. PCT/US2019/036654, filed Jun. 12, 2019; PCT Application No. PCT/US2019/057698, filed Oct. 23, 2019; PCT Application No. PCT/US2020/021596, filed Mar. 6, 2020; and U.S. application Ser. No. 16/558,224 filed Sep. 2, 2019, the disclosures of all of the aforereferenced applications are incorporated herein by reference in their entireties.

TABLE 2
Listing of DRD and exemplary ligands.

| | SEQ ID | |
|---|---|---|
| DRD Protein | NO: | Exemplary Ligands |
| Carbonic anhydrase II (CA2) (Uniprot | 30 | Celecoxib |
| ID: P00918) | | Acetazolamide |
| Human FKBP (FK506 binding protein) | 31 | Shield-1 |
| (Uniprot ID: P62942) | | |
| E. coli dihydrofolate reductase | 32 | Methotrexate (MTX) |
| (ecDHFR) (Uniprot ID: P0ABQ4) | | Trimethoprim (TMP) |
| Human dihydrofolate reductase | 33 | Methotrexate (MTX) |
| (hDHFR) (Uniprot ID: P00374) | | Trimethoprim (TMP) |
| Human estrogen receptor (ER) | 34 | Bazedoxifene |
| Uniprot ID: P03372.2) | | Raloxifene |
| Phosphodiesterase 5 (PDE5), full-length | 35 | Sildenafil; |
| (Uniprot ID: Uniprot ID O76074) | | Vardenafil; |
| | | Tadalafil |
| Phosphodiesterase 5 (PDE5), ligand | 36 | Sildenafil; |
| binding domain (Uniprot ID: Uniprot ID | | Vardenafil; |
| O76074) | | Tadalafil |

The first nucleic acid sequence encoding the transcription factor activation domain, the second nucleic acid sequence encoding the transcription factor DNA binding domain, the third nucleic acid sequence encoding the DRD, and the fourth nucleic acid sequence encoding the inhibitory RNA molecule may each be carried in a separate vector or in a single vector. Optionally, the first nucleic acid sequence encoding the transcription factor activation domain, the second nucleic acid sequence encoding the transcription factor DNA binding domain, and the third nucleic acid sequence encoding the DRD are each carried in a single vector. Optionally, the vector comprising the first nucleic acid sequence encoding the transcription factor activation domain, the second nucleic acid sequence encoding the transcription factor DNA binding domain, the third nucleic acid sequence encoding the DRD, and the fourth nucleic acid sequence encoding the inhibitory RNA molecule comprises a nucleic acid sequence of one of SEQ ID NOs: 16-19. Optionally, the first nucleic acid sequence encoding the transcription factor activation domain and the third nucleic acid sequence encoding the DRD are each carried in a single vector. Optionally, the second nucleic acid sequence encoding the transcription factor DNA binding domain and the third nucleic acid sequence encoding the DRD are each carried in a single vector. Optionally, the vector comprising the second nucleic acid sequence encoding the transcription factor DNA binding domain and the third nucleic acid sequence encoding the DRD comprises a nucleic acid sequence of SEQ ID NO: 21.

The vector can be arranged any number of ways to achieve the desired configuration of the DRD being operably linked to at least one of the transcription factor activation domain, the transcription factor DNA binding domain, or the combination of the transcription factor activation domain and the transcription factor DNA binding domain, wherein the transcription factor activation domain and the transcription factor DNA binding domain interact to form a transcription factor capable of activating transcription of the inhibitory RNA molecule upon binding to the specific nucleotide binding site. The vector optionally includes or encodes additional elements, such as a promoter sequence and other regulatory elements (enhancers, translational control elements (e.g., IRES), and/or elements that control half-life). The vector optionally comprises or can comprises nucleic acid sequences that encode elements that control translation (e.g., IRES, WPRE, and the like).

The vector can be chosen from viral vectors or non-viral vectors (e.g., plasmids, cosmids, and artificial chromosomes). Optionally, the vector is a viral vector derived from adenovirus, adeno-associated virus (AAV), alphavirus, flavivirus, herpes virus, measles virus, rhabdovirus, retrovirus, lentivirus, Newcastle disease virus (NDV), poxvirus, and picornavirus.

Vectors are optionally transferred to cells by non-viral methods by physical methods such as needles, electroporation, sonoporation, hydroporation; by carriers (such as inorganic particles (e.g., calcium phosphate, silica, gold)), and/or by chemical methods. Optionally, synthetic or natural biodegradable agents are used for delivery such agents including cationic lipids, lipid nano emulsions, nanoparticles, peptide-based vectors, or polymer-based vectors.

### Engineered Cells

Also provided herein are engineered cells comprising the set of nucleic acid sequences described above and elsewhere herein. As used herein, the terms engineered and modified are used interchangeably and mean that the cell is changed from its original state. For example, cells may be changed to express proteins they do not naturally express or changed to express more or less of the protein than they would normally express.

Optionally, the engineered cell is an immune cell. As used herein, an immune cell refers to any cell of the immune system that originates from a hematopoietic stem cell in the bone marrow. Hematopoietic stem cells gives rise to two major cell lineages, a myeloid progenitor cell lineage (which includes myeloid cells, such as monocytes, macrophages, dendritic cells, megakaryocytes and granulocytes) and a lymphoid progenitor cell lineage (which includes lymphoid cells, such as T cells, B cells, and natural killer (NK) cells). Immune cells useful in the present disclosure may be tumor infiltrating lymphocytes, T cells, natural killer T (NK-T) cells, natural killer (NK) cells, macrophages, or B cells. The immune cells may be allogeneic (i.e., the donor subject is not the recipient subject) or autologous (i.e., the donor subject is the recipient subject). Optionally, the immune cell is a T cell, an NK cell, or a tumor infiltrating lymphocyte.

Optionally, the engineered cell is a cancer cell. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancer cells may be tumors or hematological malignancies, and include but are not limited to, all types of lymphomas and leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Cells for engineering can be isolated from any biological sample, including for example, blood (e.g., umbilical cord blood or peripheral blood), bone marrow, embryonic or fetal tissue (e.g., human embryonic stem cells), or tumors. Cells can be modified cells, such as a stem cell modified to be pluripotent (e.g., an induced pluripotent stem cell (iPSC)) or chimeric antigen receptor (CAR) T cells, prior to introduction of the described nucleic acids sequences.

### Pharmaceutical Compositions

Also provided herein are pharmaceutical compositions comprising the engineered cells described above or elsewhere herein and a pharmaceutically acceptable carrier. Optionally, the engineered cells in the pharmaceutical composition are a mixed population of cells comprising a subpopulation of engineered cells and unmodified cells (e.g., cells engineered to express the set of nucleic acid sequences described herein together with untransduced or unengineered cells).

The term carrier means a compound, composition, substance, or structure that, when in combination with a compound or cells, aids, or facilitates preparation, storage, administration, delivery, effectiveness, selectivity, or any other feature of the compound or cells for its intended use or purpose. For example, a carrier can be selected to minimize any degradation of the cells in the pharmaceutical composition and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water. By pharmaceutically acceptable is meant a material that is not biologically or otherwise undesirable, which can be administered to an individual along with the cells in the pharmaceutical composition without causing unacceptable biological effects or interacting in a deleterious manner.

Optionally the pharmaceutical composition further comprises a cryoprotectant (cryopreservative). Such a cryoprotectant serves to prevent unacceptable cell lysis or damage should the cells be frozen for future use. Cryoprotectants are known in the art. Such cryoprotectants can be selected from among glycerol, ethylene glycol, propylene glycol, or dimethylsulfoxide (DMSO).

The pharmaceutical compositions described herein optionally further comprise one or more pharmaceutically acceptable excipients ((e.g., human serum albumin or polymeric materials (e.g., PEG)).

The compositions of the present disclosure can be formulated in any manner suitable for delivery. The cells can be administered in nanoparticles, poly (lactic-co-glycolic acid) (PLGA) microspheres, lipidoids, lipoplex, liposome, polymers, carbohydrates (including simple sugars), cationic lipids, or combinations thereof.

Although the descriptions of pharmaceutical compositions provided herein are principally directed to pharmaceutical compositions suitable for administration to humans, it will be understood by the skilled artisan that such compositions are generally suitable for administration to any other animal, e.g., to non-human mammals. Subjects to which administration of the pharmaceutical compositions is contemplated include, but are not limited to, agricultural animals, such as cattle, horses, chickens, and pigs; domestic animals, such as cats, dogs; or research animals such as mice, rats, rabbits, dogs, and non-human primates.

### Methods for Producing Engineered Cells

Also disclosed herein are methods of producing the engineered cell described herein comprising introducing into the cell the set of nucleic acid sequences described herein. As used herein, introducing in the context of introducing a nucleic acid sequence described herein, refers to the translocation of the nucleic acid sequence from outside a cell to inside the cell. In some cases, introducing refers to translocation of the nucleic acid sequence from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As described above, the engineered cell produced by the present methods may be an immune cell, for example, a T cell, an NK cell, or a tumor infiltrating lymphocyte. Optionally, the engineered cell is a cancer cell. As described above, cells for engineering may be isolated from any biological sample, and cells may be modified (e.g., CAR-T cells) or unmodified, prior to introduction of the nucleic acid sequences described herein.

The set of nucleic acids may be introduced into the cell using any suitable means. Optionally, the set of nucleic acids are packaged into a vector, e.g., a viral vector or non-viral vector, as described above and elsewhere herein. Optionally, the first nucleic acid sequence encoding the transcription factor activation domain, the second nucleic acid sequence encoding the transcription factor DNA binding domain, the third nucleic acid sequence encoding the DRD, and the fourth nucleic acid sequence encoding the inhibitory RNA molecule may each be carried in a separate vector or in a single vector. Optionally, the vector comprising the first nucleic acid sequence encoding the transcription factor activation domain, the second nucleic acid sequence encoding the transcription factor DNA binding domain, the third nucleic acid sequence encoding the DRD, and the fourth nucleic acid sequence encoding the inhibitory RNA molecule comprises a nucleic acid sequence of one of SEQ ID NOs: 16-19. Optionally, the first nucleic acid sequence encoding the transcription factor activation domain, the second nucleic acid sequence encoding the transcription factor DNA binding domain, and the third nucleic acid sequence encoding the DRD are each carried in a single vector. Optionally, the first nucleic acid sequence encoding the transcription factor activation domain and the third nucleic acid sequence encoding the DRD are each carried in a single vector. Optionally, the second nucleic acid sequence encoding the transcription factor DNA binding domain and the third nucleic acid sequence encoding the DRD are each carried in a single vector. Optionally, the vector comprising the second nucleic acid sequence encoding the transcription factor DNA binding domain and the third nucleic acid sequence encoding the DRD comprises a nucleic acid sequence of SEQ ID NO: 21.

Optionally, the vector is a viral vector or a non-viral vector as described above and elsewhere herein. Optionally, the vector is a viral vector derived from adenovirus, adeno-associated virus (AAV), alphavirus, flavivirus, herpes virus, measles virus, rhabdovirus, retrovirus, lentivirus, Newcastle disease virus

Vectors may be transferred to cells by non-viral methods by physical methods such as needles, electroporation, sonoporation, hydroporation; chemical carriers such as inorganic particles (e.g. calcium phosphate, silica, gold) and/or chemical methods. In some embodiments, synthetic or natural biodegradable agents may be used for delivery such as cationic lipids, lipid nano emulsions, nanoparticles, peptide based vectors, or polymer based vectors. In some embodiments, vectors may be transferred to cells by temporary membrane disruption, for example, by high speed cell deformation.

### Methods of Delivering a Regulatable Inhibitory RNA Molecule

Disclosed herein are methods of delivering a regulatable inhibitory RNA molecule to a subject comprising administering to the subject the set of nucleic acid sequences, the engineered cells, or the pharmaceutical compositions described above and elsewhere herein. As used herein, subject is not limited to human subjects, and also includes rodent (e.g., mouse or rat), canine, feline, avian, or monkey subjects.

The methods may further comprise controlling the dose or duration of administration of an inhibitory RNA molecule to a subject. Optionally, the DRD operably linked to at least one of the transcription factor activation domain, the transcription factor DNA binding domain, or the combination of the transcription factor activation domain and the transcription factor DNA binding domain, is stabilized upon exposure to a paired ligand, the DRD-TF is stabilized. The stabilized DRD-TF is then able to bind to the specific nucleotide binding site to which the transcription factor DNA binding domain binds, and thus regulate transcription of the nucleic acid sequence encoding the inhibitory RNA molecule as described herein. In the absence of the exogenous stabilizing ligand, the DRD-TF is degraded and unable to activate transcription of the inhibitory RNA molecule. Thus, both the amount and the timing of inhibitory RNA molecule expression can be controlled by administering the ligand to the subject in accordance with the present method.

In certain examples, the present method comprises alternatively administering to the subject varying selected amounts of ligand, to achieve varying selected activity levels of DRD-TF and thus inhibitory RNA molecule expression. For example, the method optionally further comprises administering to the subject a selected amount of one or more paired ligands to deliver a selected activity of the inhibitory RNA molecule to the subject. The one or more ligands can be delivered to achieve continuous or intermittent inhibitory RNA molecule activity in the subject. Inhibitory RNA molecule activity may be a substantially consistent level of activity, or the level of activity may be modulated. Intermittent activity, between the off-state and on-state includes modulating activity between the off-state and a substantially consistent on-state, or between the off-state and varying on-state activity levels. A higher dose or longer duration of administration of the ligand is administered when more activity of the inhibitory RNA molecule is desired, and reduction or elimination of the ligand dose is chosen when less activity is desired.

The dose and duration of administration of the one or more ligands and the resulting activity of the inhibitory RNA molecule may be selected to avoid unacceptable or undesired side effects or toxicity in the subject. Dosages of ligand and schedules for administering the dosages of ligand may be determined empirically by one skilled in the art based on the amount, activity, or effect of the resulting inhibitory RNA molecule. The ranges for administration of the one or more ligands range from any amount above zero to a saturating dose and the resulting inhibitory RNA molecule expression ranges from a basal level to a maximal level, optionally with a sufficient dynamic range that allows for the desired dose-response to the ligand and concomitant activity range for the inhibitory RNA molecule activity (e.g., the range of difference in off-state and maximum inhibitory RNA molecule activity would result from at least a 10 fold range of ligand). This sufficient dynamic range allows for fine tuning and a dose response curve that is not unacceptably steep. In certain embodiments, the dosage or frequency of administration of ligand and resulting abundance and activity of inhibitory RNA molecule is chosen to avoid, mitigate against, or limit unacceptable or undesired adverse side effects and will vary with the age, condition, and/or sex of the subject, and type of condition being treated, the extent of the condition, or, and whether other therapeutic agents are included in the treatment regimen. Guidance can be found in the literature for appropriate dosages for given classes of ligands.

As used herein, administering is understood to mean providing an agent (such as a ligand) to a target (such as a DRD) such that the agent and target may come into contact with one another. Administration can be in vitro, ex vivo, or in vivo, whereby the agent is added to cell culture medium or administered to the subject. As a non-limiting example, administering also includes providing the ligand to a cell, wherein the DRD is located intracellularly, such that the ligand reaches the cytoplasm or the nucleus or other cellular organelles. Similarly, administration can be in vivo, for example by administering a ligand to a subject such that the ligand reaches a cell or the DRD contained on the surface or in the interior of the cell. In each case, the ligand needs to reach a minimum intracellular concentration to exert its stabilizing effect on the DRD.

### Methods for Treatment

Also provided herein are methods for treating a disease in a subject in need thereof comprising administering to the subject the set of nucleic acid sequences, the engineered cells, or the pharmaceutical compositions described above and elsewhere herein. Optionally, the disease is a neurodegenerative disease, for example, Alzheimer's disease, Huntington's disease, Parkinson's disease, multiple system atrophy, amyotrophic lateral sclerosis, ataxia, Lewy body disease, frontotemporal dementia, and vascular dementia. Optionally, the neurodegenerative disease is Alzheimer's disease or Huntington's disease. Optionally, the disease is a degenerative or inflammatory joint disease, for example osteoarthritis, rheumatoid arthritis, spondyloarthritis, fibromyalgia, systemic lupus, or gout. Optionally, the disease is an ocular disease, for example age-related macular degeneration.

As used herein, the terms treatment or treating denote an approach for obtaining a beneficial or desired result. Such beneficial or desired clinical results include, but are not limited to, one or more of the following: reducing the proliferation of (or destroying) diseased cells, promoting an anti-disease immune response, decreasing symptoms resulting from the disease, increasing the quality of life of those suffering from the disease, decreasing the dose of other medications required to treat the disease, delaying the progression of the disease, and/or prolonging survival of individuals.

As described above, the present methods may further comprise administering to the subject varying selected amounts of ligand, to achieve varying selected activity levels of DRD-TF and thus inhibitory RNA molecule expression. The recipient subject is optionally monitored for the outcome of the treatment. For example, for the progression of the disease using imaging or other modalities, the number of diseased cells in a sample, or the symptoms of the disease. If the desired end point is achieved (e.g., showing successful treatment of the disease), the ligand can be reduced or discontinued so as to reduce or eliminate the amount or activity of the inhibitory RNA molecule. Similarly, if the subject develops an undesired reaction to the treatment, for example, an allergic reaction, or other adverse effect from the treatment, the ligand can be reduced or discontinued. Administration of the ligand can optionally be increased or restarted after the reduction or discontinuation.

### Methods for Regulation of Immune Cells

Also provided herein are methods for regulation of an immune cell comprising administering to the immune cell the set of nucleic acid sequences described above or elsewhere herein. The present method may be useful, for example, to prevent excessive immune responses.

Immune cell regulation resulting from the present methods may be observed by any suitable method. For example, immunofluorescence staining or flow cytometry may be used to observe immune cell activity and surface marker expression.

As described above, the immune cell is optionally a T cell, an NK cell, or a TIL. Optionally the immune cell is a T cell. Optionally, the T cell is modified to express a chimeric antigen receptor (CAR). Optionally, the inhibitory RNA molecule of the set of nucleic acid sequences described herein downregulates transcription of a T cell receptor signaling gene. Optionally, the T cell receptor signaling gene is selected from the group consisting of Lck, zeta-chain-associated protein kinase-70 (ZAP70), unc119, or linker for activation of T cells (LAT).

### Kits

Provided herein is a kit comprising the set of nucleic acid sequences or the engineered cell described above and elsewhere herein, and a ligand to which the DRD of the set of nucleic acid sequences or the engineered cell wherein the DRD is responsive. Optionally, the ligand is Acetazolamide (ACZ), Methotrexate (MTX), or Trimethoprim (TMP).

Optionally, the set of nucleic acid sequences or the engineered cell provided herein will be in solution in a diluent. The kit may have containers containing a diluent, solubilizer, emulsifier, and/or preservative to be used with the methods disclosed herein. Optionally, the kits described further comprise one or more devices for therapeutic delivery of the set of nucleic acid sequences or the engineered cell to the subject according to the methods described herein. The one or more devices for therapeutic delivery may be a carrier container being compartmentalized to receive in close confinement one or more containers such as vials, tubes, and the like, with one or more of the containers comprising the set of nucleic acid sequences or the engineered cell provided herein. The kit may also have containers containing buffer(s) and/or a container comprising a reporter-means, such as a biotin-binding protein, such as avidin or streptavidin, bound to a reporter molecule, such as an enzymatic or fluorescent label.

The kit may have containers containing a diluent, solubilizer, emulsifier, and/or preservative to be used with the methods disclosed herein. The kit optionally contains a diluent formulated for intravenous infusion of the set of nucleic acid sequences or the engineered cell provided herein.

Optionally, the kit comprises the set of nucleic acid sequences or the engineered cell provided herein in a single unit dosage form or as separate unit doses. The dose and form of the unit dose (e.g., tablet, capsule, immediate release, delayed release, etc.) can determined by one skilled in the art depending upon, for example, the intended route of administration, delivery format and desired dosage. Optionally, the kit may include the set of nucleic acid sequences or the engineered cell provided herein in a single-dose administration unit. Optionally, kits containing single or multi-chambered pre-filled syringes comprising the set of nucleic acid sequences or the engineered cell are included.

Disclosed are materials, compositions, and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of components of the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. A number of embodiments have been described. Nevertheless, it will be understood that various modifications may be made. Accordingly, other embodiments are within the scope of the claims.

### EXAMPLES

The following examples are provided by way of illustration only and not by way of limitation. Those of skill in the art will readily recognize a variety of non-critical parameters that could be changed or modified to yield essentially the same or similar results.

### Example 1. Determining an Optimal Promoter for Regulated RNAi

In a first set of experiments, constructs TXN-036 (SEQ ID NO: 18), TXN-037 (SEQ ID NO: 19), and TXN-038 (SEQ ID NO: 20) were constructed to determine the optimal promoter for expressing shRNA. Each of constructs TXN-036, -037, and -038 contained a different Ubiquitin promoter variant to regulate transcription of a nucleic acid sequence encoding a shRNA molecule targeting PEST-tagged enhanced green fluorescent protein (PEST-tagged EGFP). TXN-036 contained an HubC promoter with a minimal termination signal and shRNA placed at +1 (the first downstream nucleosome) from the promoter, referred to herein as an HubC “short” promoter (see, e.g., Giering et al., Molecular Therapy, 2008); TXN-037 contained an UbiC promoter (see, e.g., Lebbink et al., PLOS One, 2011); and TXN-038 contained a UbiC promoter plus a hairpin mimicking a human micro RNA miR-30 structure in a downstream intron (see, e.g., Zhou et al., Nucleic Acids Res., 2005). TXN-036, -037, and -038 are further described in Table 1 above.

Fluorescence was measured using flow cytometry. TXN-037, which employed a UbiC promoter without the hairpin in a downstream intron, had the highest amount of GFP knockdown and was used for further experiments unless otherwise specified. GFP knockdown was not observed from TXN-036, which employed a HubC short promoter.

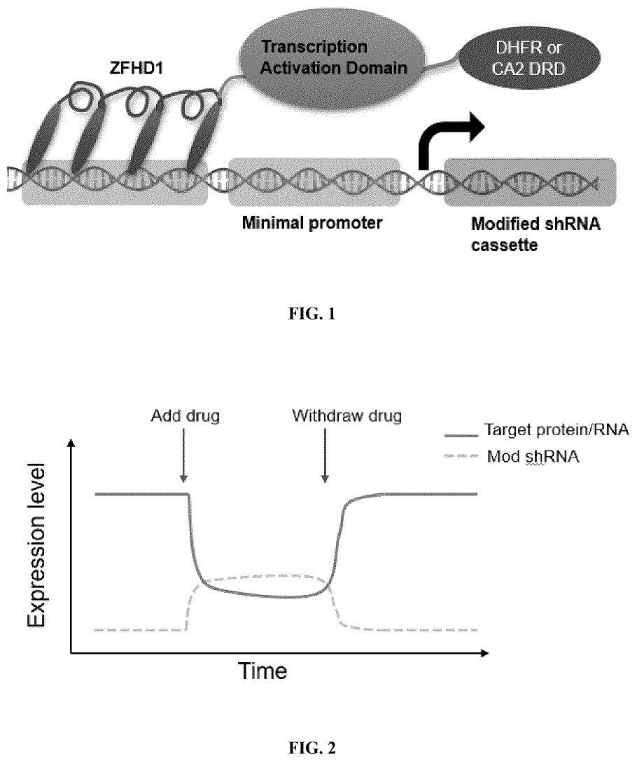

FIG. 1 shows a schematic of a transcription cassette comprising the components described herein. FIG. 2 shows an exemplary graph of ligand (drug)-regulated shRNA expression, and consequent target protein or RNA expression

### Example 2. Assessing DRD-Regulated RNAi-Based Regulation of GFP in Dual Vector Systems

In this series of experiments, DRD-regulated RNAi-based regulation of GFP was assessed in HEK293 T cells. HEK293 T cells were transduced with the TXN-041 (SEQ ID NO: 21) construct, in which ZFHD1 was operably linked to a hDHFR DRD, and sorted by cell type based on Thy 1.2 expression. Next, the HEK293 T cells were transduced with the TXN-051 (SEQ ID NO: 22) construct containing a UbiC promoter upstream of mCherry-tagged shRNA targeting GFP. The HEK293 T cells were treated with trimethoprim (TMP) for 3 days at 0, 50, and 300 μl. Cells were analyzed by flow cytometry for mCherry and GFP expression. TXN-041 and -051 are described in Table 1 above.

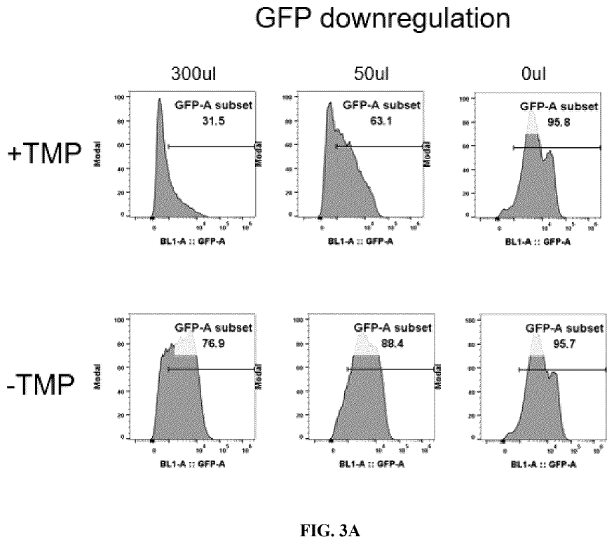

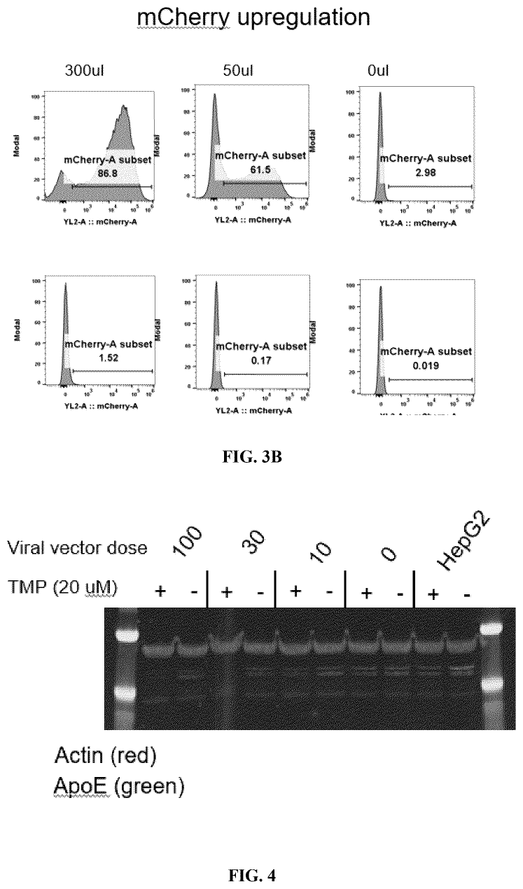

As shown in FIG. 3A, GFP expression in TXN-041 and TXN-051-transduced HEK293T cells was downregulated upon TMP addition, and further decreased as TMP dose increased from 50 to 300 μl. Similarly, as shown in FIG. 3B, mCherry expression, and thus shRNA expression, in TXN-041 and TXN-051-transduced HEK293T cells was upregulated upon TMP addition, and further increased as TMP dose increased from 50 to 300 μl.

### Example 3. Assessing DRD-Regulated RNAi-Based Regulation of ApoE in Dual Vector Systems

Next, ApoE knockdown was assessed using the DRD-regulated RNAi-based regulation described herein. HepG2 cells, derived from a human liver cancer cell line, were transduced with TXN-041 (SEQ ID NO: 21) at a 30% transduction rate in order to obtain a single vector copy number, and sorted by cell type based on Thy 1.2 expression. Next, the HepG2 cells were transduced with the ApoE_Ri-004 (SEQ ID NO: 23) construct, described in Table 1 above, containing a UbiC promoter upstream of mCherry-tagged shRNA targeting ApoE, at 0, 10, 30, or 100 μl virus/well. Cells were treated with TMP for 4 days at 20 μM TMP. and with Brefeldin A for 90 minutes.

A subset of cells were harvested for western blot, trypsinized, and centrifuged at 300G for 5 minutes. Supernatant was aspirated and the remaining cell pellet was frozen at −80° C. Cells were treated with the antibodies in Table 3 below for western blot analysis.

TABLE 3
Antibodies used in Example 3.

| | Dilution | Purpose |
|---|---|---|
| Primary Antibody | | |
| Mouse anti-actin (catalog # 3700) | 1:1000 | cell signaling |
| Rabbit anti-ApoE (catalog # 13366) | 1:1000 | cell signaling |
| Secondary Antibody | | |
| LICOR IRDye 800CW (green) | 1:2000 | anti-rabbit |
| LICOR IRDye 680RD (red) | 1:2000 | anti-mouse |

The remaining cells were stained for flow cytometry analysis. Cells were surface stained for Thy 1.2 with BV421, Brilliant Violet 421™ anti-mouse CD90.2 (Thy-1.2) Antibody, Rat IgG2a, κ (Biolegend, catalog #140327) at 1:100 and intracellular staining for ApoE with ApoE (pan) (D719N) Rabbit mAb (Alexa Fluor® 488 Conjugate) (CST catalog #76772) at 1:100. Cells were processed for flow cytometry using the Invitrogen Fix and Perm Kit (Thermo Fisher) according to the manufacturer's instructions.

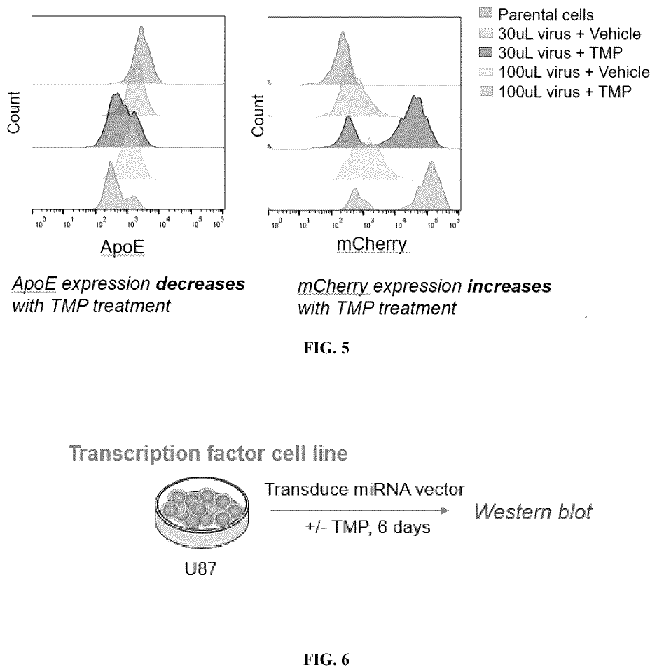

Western blot analysis showed apoE expression was decreased with increasing viral vector dose and TMP treatment (FIG. 4), which was further confirmed by flow cytometry analysis (FIG. 5, left panel). Conversely, mCherry, and thus shRNA, expression increased with increasing viral vector dose and TMP treatment (FIG. 5, right panel).

### Example 4. Assessing DRD-Regulated RNAi-Based Regulation of HTT in Dual Vector Systems

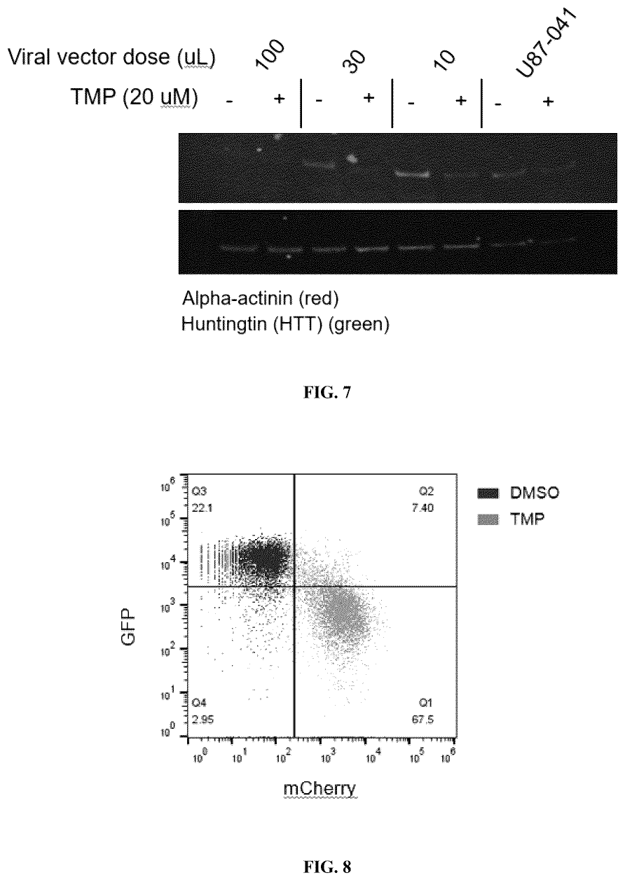

HTT knockdown was assessed using the DRD-regulated RNAi-based regulation described herein. The experimental design is depicted in FIG. 6. Briefly, U87 cells, derived from a human glioma cell line, were transduced with TXN-041 (SEQ ID NO: 21) at a 30% transduction rate in order to obtain a single vector copy number, and sorted by cell type based on Thy 1.2 expression. Next, the cells were transduced with the HTT-001 (SEQ ID NO: 24) construct, containing a UbiC promoter upstream of mCherry-tagged shRNA targeting HTT, at 0, 10, 30, or 100 μl virus/well. Cells were treated with TMP for 6 days at 20 μM TMP.

A subset of cells were harvested for western blot, trypsinized, and centrifuged at 300G for 5 minutes. Supernatant was aspirated and the remaining cell pellet was frozen at −80° C. Cells were treated with the antibodies in Table 4 below for western blot analysis.

TABLE 4
Antibodies used in Example 4.

| | Dilution | Purpose |
|---|---|---|
| Primary Antibody | | |
| mouse alpha actinin (catalog # | 1:1000 | cell signaling |
| 69758S) | | |
| rabbit huntingtin (catalog # 5656S) | 1:1000 | cell signaling |
| Secondary Antibody | | |
| LICOR IRDye 800CW (green) | 1:2000 | anti-rabbit |
| LICOR IRDye 680RD (red) | 1:2000 | anti-mouse |

Western blot analysis showed HTT expression was decreased with increasing viral vector dose and TMP treatment (FIG. 7).

### Example 5. Assessing DRD-Regulated RNAi-Based Regulation of GFP in a Single Vector System

Next, DRD-regulated RNAi-based regulation of GFP in HEK293 T cells using a single vector system was assessed. GFP-expressing HEK293 T cells were generated by transducing HEK293 T cells with EGFP-038 (SEQ ID NO: 39), comprising a destabilized PEST-tagged GFP, at a 30% transduction rate in order to obtain a single vector copy number. Cells were then sorted based on Thy 1.1 marker expression to create a stable cell pool. Next, the GFP-transduced HEK239T cells were transduced with the TXN-059 (SEQ ID NO: 25) construct at 10, 30, 100, or 250 μl virus/well, and sorted by cell type based on Thy 1.2 expression. Cells were treated with trimethoprim (TMP) for 3 days at 20 μM. Cells were analyzed by flow cytometry for mCherry and GFP expression. EGFP-038 and TXN-059 are described in Table 1 above.

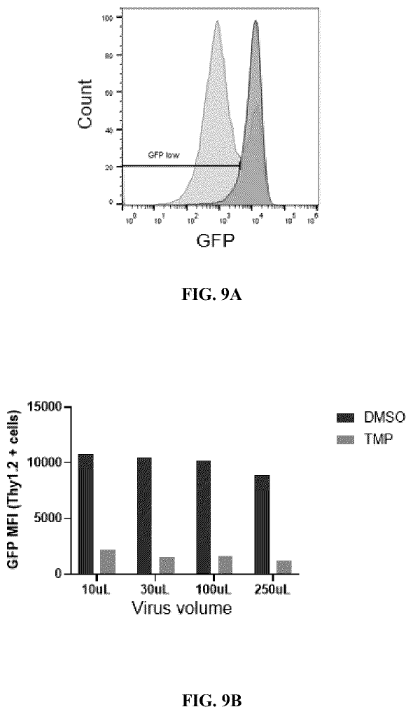

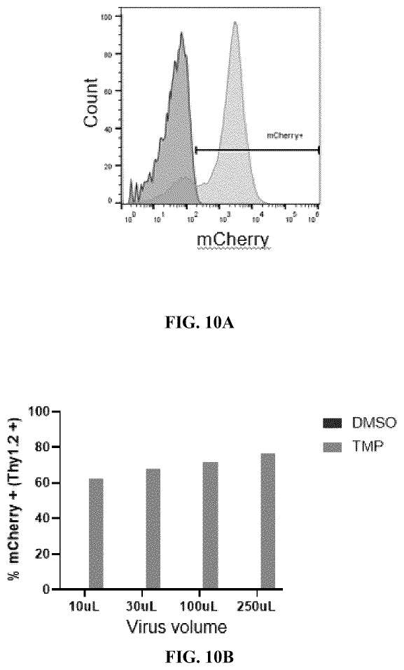

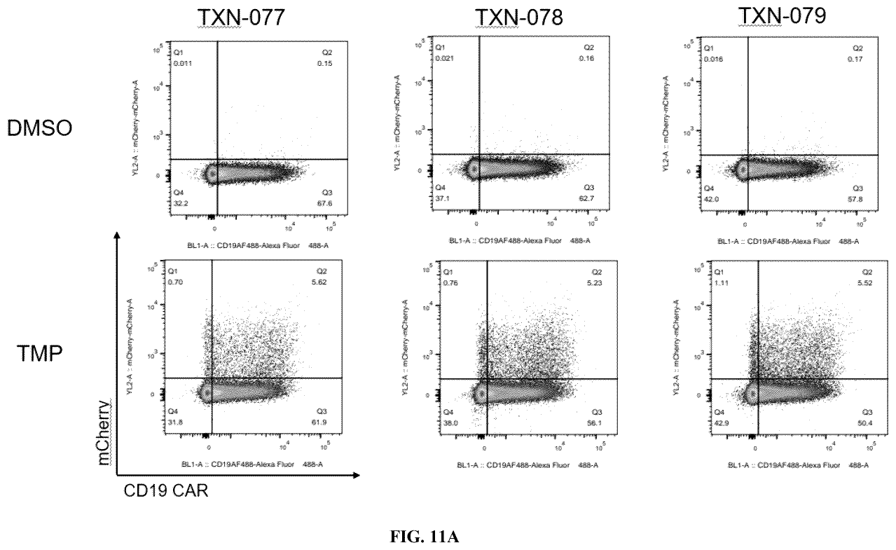

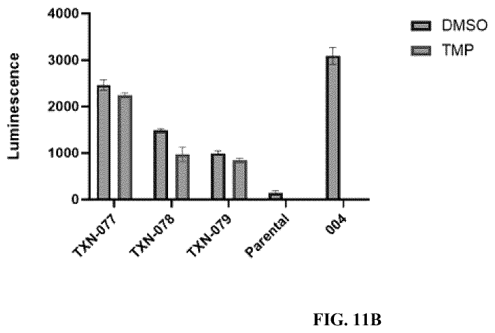

GFP expression in EGFP-038 and TXN-059-transduced HEK293T cells was downregulated upon TMP addition, and decreased as viral vector dose increased from 10 to 250 μl (FIGS. 8 and 9A-B). Similarly, as shown in FIG. 10A-B, mCherry expression, and thus shRNA expression, in EGFP-038 and TXN-059-transduced HEK293T cells was upregulated upon TMP addition, and increased as viral vector dose increased from 10 to 250 μl.

### Example 6. Assessing DRD-Regulated RNAi-Based Regulation of TCR Signaling in a Single Vector System

Next, DRD-regulated RNAi-based regulation of T cell receptor (TCR) signaling genes ZAP70, LAT, and Lck in T cells using a single vector system was assessed. An NFAT-luciferase reporter T cell line was generated by transducing CD8+TCR knockout NFAT-luciferase reporter Jurkat T cells (BPS Bioscience, catalot #78757) with the TCR-004 construct (SEQ ID NO: 29).

The TCR-004 NFAT-luciferase reporter Jurkat T cells were then transduced with constructs described in Table 5 below, at 10 or 30 μl virus/well. After 48 hours, cells were prepared for flow cytometry analysis by staining with eBioscience™ Fixable Viability Dye eFluor™ 780 antibody at 1:1000, G4S Linker (E702V) Rabbit mAb (Alexa Fluor® 488 Conjugate) #50515, at 1:50, and Brilliant Violet 421™ anti-human CD3 Antibody at 1:100. Cells were treated with either 20 μM TMP or DMSO. Cells were gated by CD19 expression.

TABLE 5
Constructs used in Example 6.

| | SEQ | Gene |
|---|---|---|
| Name | ID NO: | target |
| TXN-077 (12xZFHD1 YB-TATA-mCherry-shRNA | 17 | ZAP70 |
| targeting ZAP70-EFS-ZFHD1-hDHFR-MND-CD19 CAR) | | |
| TXN-078 (12xZFHD1 YB-TATA-mCherry-shRNA | 18 | ZAP70 |
| targeting ZAP70-EFS-ZFHD1-hDHFR-MND-CD19 CAR) | | |
| TXN-079 (12xZFHD1 YB-TATA-mCherry-shRNA | 19 | ZAP70 |
| targeting ZAP70-EFS-ZFHD1-hDHFR-MND-CD19 CAR) | | |
| TXN-080 (12xZHFD1-YB-TATA-mCherry-shRNA | 34 | LAT |
| targeting LAT-EFS-Nls-Zfhd1-ierNLS-p65- | | |
| GGSGGGSG-hDHFR(134)-MND-CD19CAR) | | |
| TXN-081 (12xZHFD1-YB-TATA-mCherry-shRNA | 35 | LAT |
| targeting LAT-EFS-Nls-Zfhd1-ierNLS-p65- | | |
| GGSGGGSG-hDHFR(134)-MND-CD19CAR) | | |
| TXN-082 (12xZHFD1-YB-TATA-mCherry-shRNA | 36 | LAT |
| targeting LAT-EFS-Nls-Zfhd1-ierNLS-p65- | | |
| GGSGGGSG-hDHFR(134)-MND-CD19CAR) | | |
| TXN-123 (12xZHFD1-YB-TATA-mCherry-shRNA | 37 | Lck |
| targeting Lck-EFS-Nls-Zfhd1-ierNLS-p65- | | |
| GGSGGGSG-hDHFR(134)-MND-CD19CAR) | | |
| TXN-124 (12xZHFD1-YB-TATA-mCherry-shRNA | 38 | Lck |
| targeting Lck-EFS-Nls-Zfhd1-ierNLS-p65- | | |
| GGSGGGSG-hDHFR(134)-MND-CD19CAR) | | |
| TXN-125 (12xZHFD1-YB-TATA-mCherry-shRNA | 39 | Lck |
| targeting Lck-EFS-Nls-Zfhd1-ierNLS-p65- | | |
| GGSGGGSG-hDHFR(134)-MND-CD19CAR) | | |

After 7 days of TMP treatment, a subset of cells underwent an APC co-culture assay. SW260 colorectal cancer cells were plated at 1*10\hat{ }5 mL cells per 100 μL per plate. Treated and experimental wells were treated 50 μL of 40 μM C1-28 peptide. Cells were incubated for 1 hour at 37° C. without CO2. The TCR-004 NFAT-luciferase reporter Jurkat T cells transduced with the constructs in Table 5 were added to the culture. The ONE-Step® luciferase assay system was used to assess NFAT luminescence. Briefly, Component A and Component B from ONE-Step® luciferase assay (BPS Bioscience, San Diego, CA) was thawed in a 37° C. water bath. Component A and Component B were added at a 1:100 ratio to make the working solution. 100 μL of spent media was removed from culture without disturbing the cell pellet, and set aside. 100 μL of the working solution was added to the culture medium, rocked on a plate shaker at 300 rpm for 30 minutes, while covered with foil. Luminescence was read using a Biotek Synergy H1 microplate reader.

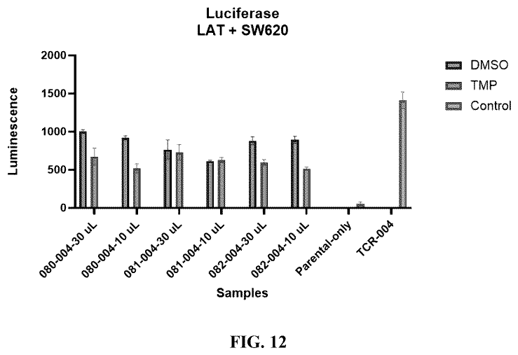

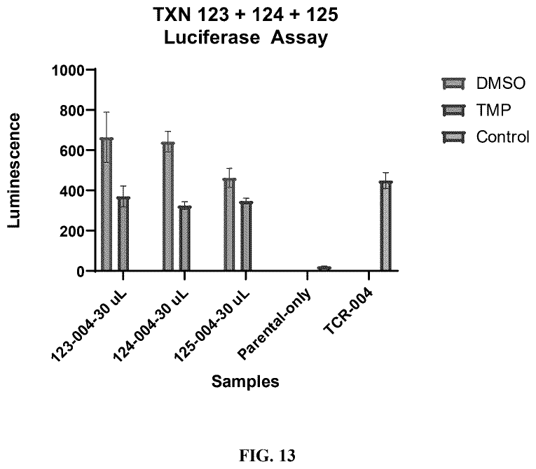

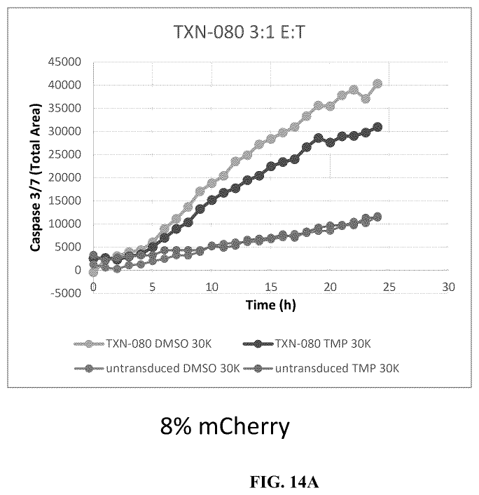

As shown in FIG. 11A, mCherry, and thus shRNA, expression was increased with TMP treatment in cells transduced with TXN-077, -078, and -079. Consequently, ZAP70 knockdown decreases TCR signaling, assessed through NFAT-luciferase activity which decreased upon TMP treatment in cells transduced with 30 μL TXN-078 (FIG. 11B). As shown in FIG. 12, LAT knockdown decreases TCR signaling, assessed through NFAT-luciferase activity which decreased upon TMP treatment in cells transduced with 10 μL and 30 μL TXN-080 and TXN-082. As shown in FIG. 13, Lck knockdown decreases TCR signaling, assessed through NFAT-luciferase activity which decreased upon TMP treatment in cells transduced with 30 μL TXN-123, -124, and -125. Transduction efficiency was assessed for each of TXN-123, -124, and -125 (FIG. 13). These data show knockdown of ZAP70, LAT, or Lck by regulated shRNA interferes with TCR signaling.

### Example 7. Assessing Cytotoxicity of CAR-T Cells Using DRD-Regulated RNAi-Based Regulation of TCR Signaling

Next, cytotoxicity of CAR-T cells using DRD-regulated RNAi-based regulation of TCR signaling was assessed using a CAR-T cell and Nalm6 leukemia cell line co-culture. Human T cells were thawed and activated with a 3:1 ratio of CD3/CD28 beads. T cells were transduced with TXN-080, -081, or -082 (SEQ ID NOs: 34, 35, and 36, respectively), targeting LAT expression. Cells were treated with either 20 μM TMP or DMSO. Cells were expanded and TMP or DMSO treatment maintained over 5-7 days.

Transduction efficiency was analyzed by flow cytometry. Briefly, 10⁰ cells were spun down and resuspended in 100 μL staining buffer (Alexa Fluor® 488 Conjugate) (CST catalog #76772) at 1:50 and LIVE/DEAD™ Fixable Far Red Dead Cell Stain Kit at 1:1000. Cells were rested for 20 minutes at room temperature, washed, and resuspended in staining buffer or PBS for flow cytometry analysis on an Attune NxT acoustic focusing flow cytometer (Thermo Fisher).

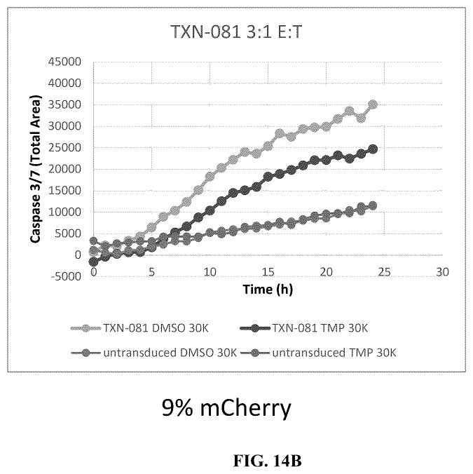

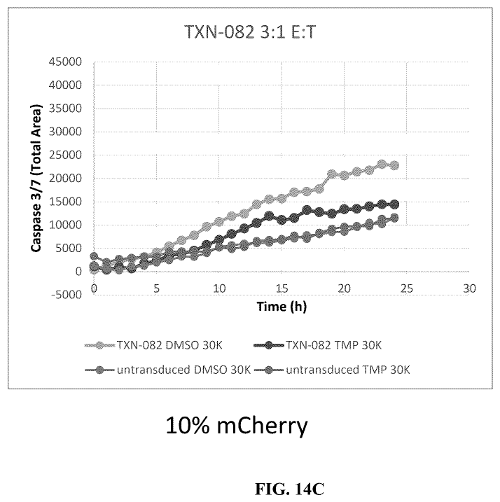

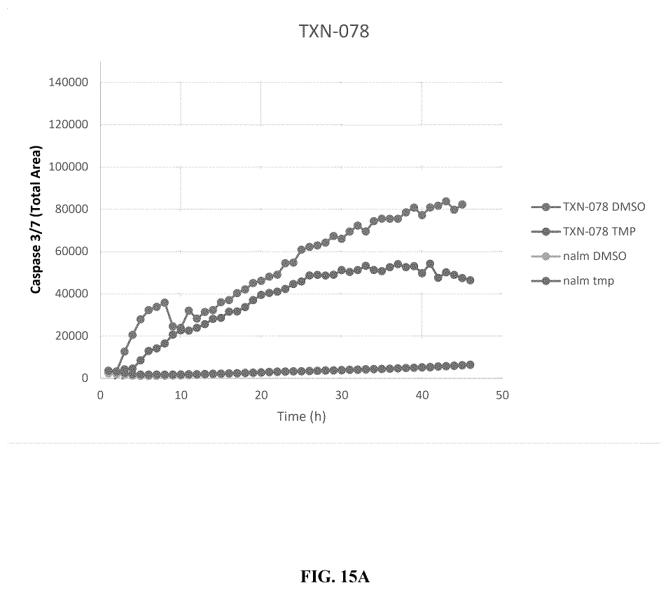

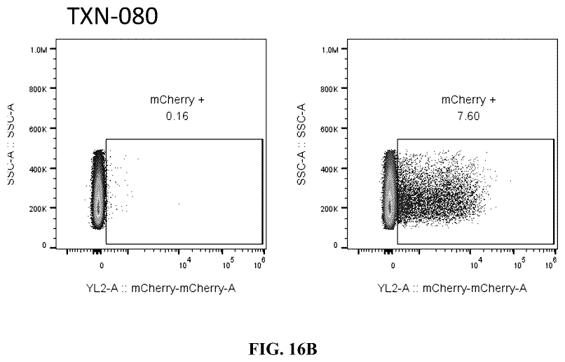

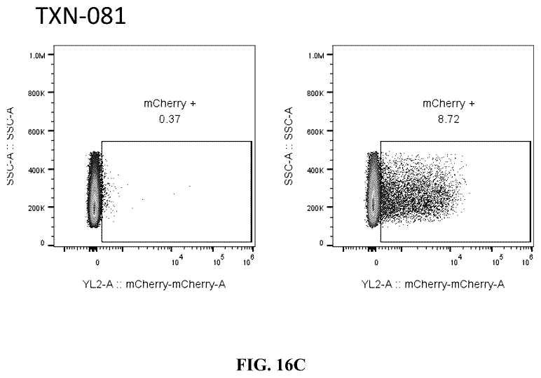

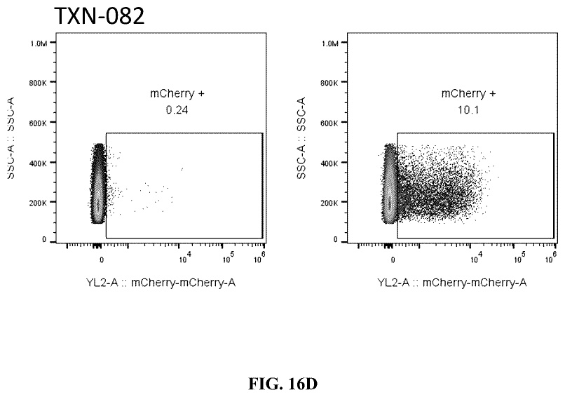

For the cytotoxicity assay, a tissue-culture treated 96-well flat bottom plate was coated with 50 μL of poly-L-Lysine (at stock concentration) and incubated for 1 hour at room temperature before aspiration removal. 1*10\hat{ }6 Nalm6 cells expressing CD19 were resuspend in 20 mL of T-cell media as described above for a concentration of 0.1*10\hat{ }6 cells per well. 100 μL of target cells were added after poly-L-Lysine removal and rested for 40 minutes at room temperature. CD19 CAR-T cells were transduced with TXN-078, -080, -081, -082, -124, and -076 (SEQ ID NOs: 18, 34, 35, 36, 38, and 33, respectively) and diluted in T-cell media as described above. 100 μL of CAR-T cells were added on top of target cells at a 3:1 (FIGS. 14A-14C) or 10:1 (FIGS. 16A-16D) CAR-T cell to target cell ratio (effector:target or E:T ratio). A 20× concentration stock of DMSO or TMP was made in fresh T cell media and 10 μL of DMSO or TMP stock was added to appropriate wells. Incucyte® Caspase-3/7 Green Dye (Sartorius) was diluted at 1:20 and 2 μL of diluted dye was added to each well for a final concentration of 1:2,000. Green and phase fluorescence was evaluated in Incucyte® (Sartorius) every hour at a 10× objective for 48 hours.

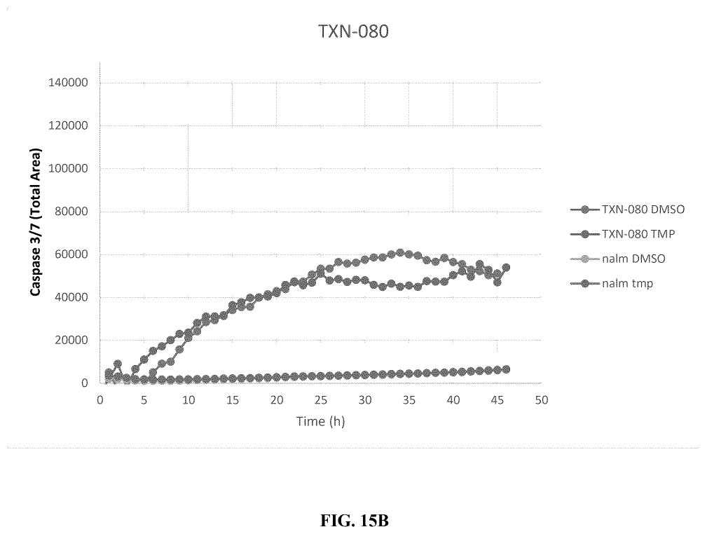

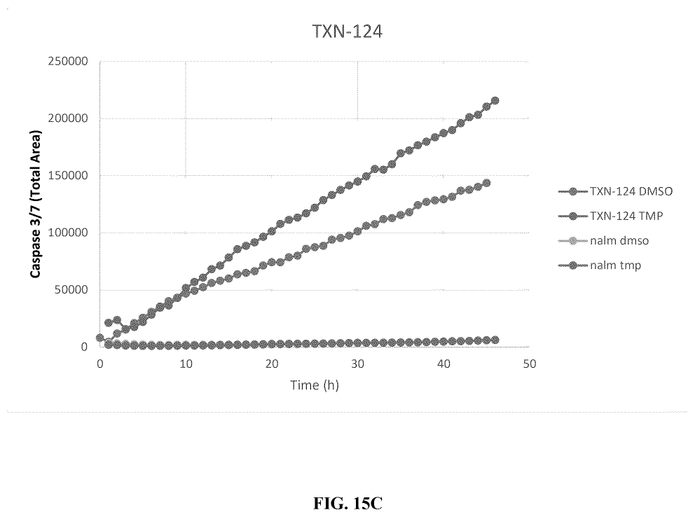

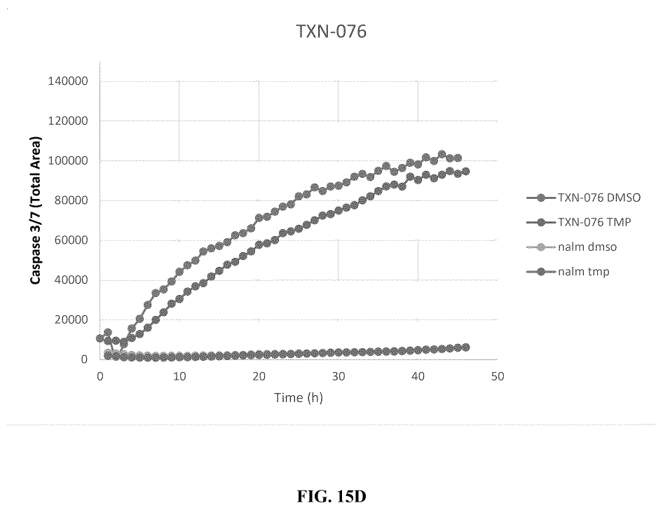

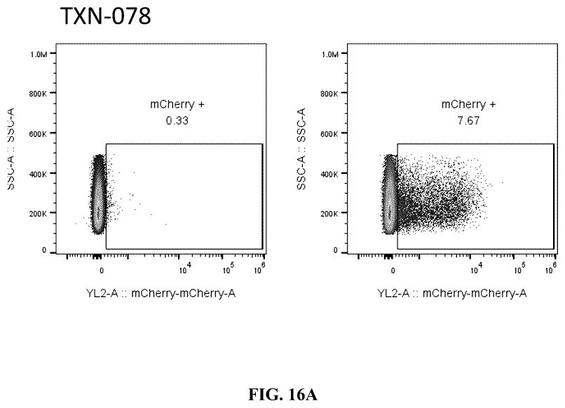

As shown in FIGS. 14A-14C, some regulation was seen in each of TXN-080, -081, and -082 at a 3:1 E:T ratio. Cells transduced with TXN-078 are not shown in FIGS. 14A-C due to technical issues with plating in the co-culture assay, but are shown below in FIG. 16A. TXN-082 demonstrated the highest basal activity knockdown (FIG. 14C). At a 10:1 E:T ratio, some regulation was seen in TXN-078, -080, -124, and -076 (FIGS. 15A-15D). As shown in FIGS. 16A-16D, mCherry, and thus ZAP70 and LAT shRNA, expression was increased with TMP treatment in cells transduced with TXN-078, -080, -081, and -082. These data show knockdown of ZAP70 or LAT by regulated shRNA can interfere with CAR signaling decreasing cell killing activity.

INFORMAL SEQUENCE LISTING

| | | SEQ ID |
|---|---|---|
| DESCRIPTION | NUCLEIC ACID SEQUENCE | NO: |
| Promoter UbiC | GGCCTCCGCGCCGGGTTTTGGCGCCTCCCGCGGGCGCCCCC | 1 |
| | CTCCTCACGGCGAGCGCTGCCACGTCAGACGAAGGGCGCA | |
| | GCGAGCGTCCTGATCCTTCCGCCCGGACGCTCAGGACAGC | |
| | GGCCCGCTGCTCATAAGACTCGGCCTTAGAACCCCAGTATC | |
| | AGCAGAAGGACATTTTAGGACGGGACTTGGGTGACTCTAG | |
| | GGCACTGGTTTTCTTTCCAGAGAGCGGAACAGGCGAGGAA | |
| | AAGTAGTCCCTTCTCGGCGATTCTGCGGAGGGATCTCCGTG | |
| | GGGCGGTGAACGCCGATGATTATATAAGGACGCGCCGGGT | |
| | GTGGCACAGCTAGTTCCGTCGCAGCCGGGATTTGGGTCGC | |
| | GGTTCTTGTTTGTGGATCGCTGTGATCGTCACTTGGT | |
| HUbC promoter | GATCTGGCCTCCGCGCCGGGTTTTGGCGCCTCCCGCGGGCG | 2 |
| short | CCCCCCTCCTCACGGCGAGCGCTGCCACGTCAGACGAAGG | |
| | GCGCAGGAGCGTCCTGATCCTTCCGCCCGGACGCTCAGGA | |
| | CAGCGGCCCGCTGCTCATAAGACTCGGCCTTAGAACCCCA | |
| | GTATCAGCAGAAGGACATTTTAGGACGGGACTTGGGTGAC | |
| | TCTAGGGCACTGGTTTTCTTTCCAGAGAGCGGAACAGGCG | |
| | AGGAAAAGTAGTCCCTTCTCGGCGATTCTGCGGAGGGATC | |
| | TCCGTGGGGCGGTGAACGCCGATGATTATATAAGGACGCG | |
| | CCGGGTGTGGCACAGCT | |
| UbiC promoter + | CGGCCTCCGCGCCGGGTTTTGGCGCCTCCCGCGGGCGCCCC | 3 |
| miR-30-like hairpin | CCTCCTCACGGCGAGCGCTGCCACGTCAGACGAAGGGCGC | |
| | AGCGAGCGTCCTGATCCTTCCGCCCGGACGCTCAGGACAG | |
| | CGGCCCGCTGCTCATAAGACTCGGCCTTAGAACCCCAGTAT | |
| | CAGCAGAAGGACATTTTAGGACGGGACTTGGGTGACTCTA | |
| | GGGCACTGGTTTTCTTTCCAGAGAGCGGAACAGGCGAGGA | |
| | AAAGTAGTCCCTTCTCGGCGATTCTGCGGAGGGATCTCCGT | |
| | GGGGCGGTGAACGCCGATGATTATATAAGGACGCGCCGGG | |
| | TGTGGCACAGCTAGTTCCGTCGCAGCCGGGATTTGGGTCGC | |
| | AGTTCTTGTTTGTGGATCGCTGTGATCGTCACTTGGTGAGT | |
| | AGCGGGCTGCTGGGCTGGCCGGGGCTTTCGTGGCCGCCGG | |
| | GCCGCTCGGTGGGACGGAGGCGTGTGGAGAGACCGCCAAG | |
| | GGCTGTAGTCTGGGTCCGCGAGCAAGGTTGCCCTGAACTG | |
| | GGGGTTGGGGGGAGCGCAGCAAAATGGCGGCTGTTCCCGA | |
| | GTCTTGAATGGAAGACGCTTGTGAGGCGGGCTGTGAGGTC | |
| | GTTGAAACAAGGTGGGGGGCATGGTGGGCGGCAAGAACCC | |
| | AAGGTCTTGAGGCCTTCGCTAATGCGGGAAAGCTCTTATTC | |
| | GGGTGAGATGGGCTGGGGCACCATCTGGGGACCCTGACGT | |
| | GAAGTTTGTCACTGACTGGAGAACTCGGTTTGTCGTCTGTT | |
| | GCGGGGGCGGCAGTTATGGCGGTGCCGTTGGGCAGTGCAC | |
| | CCGTACCTTTGGGAGCGCGCGCCCTCGTCGTGTCGTGACGT | |
| | CACCCGTTCTGTTTGCTGTTGACAGTGAGCGATCAAGCTGA | |
| | CCCTGAAGTTCATTAGTGAAGCCACAGATGTAATGAACTTC | |
| | AGGGTCAGCTTGCCTGCCTACTGCCTCGGACTTCAAGGGGG | |
| | CTTATAATGCAGGGTGGGGCCACCTGCCGGTAGGTGTGCG | |
| | GTAGGCTTTTCTCCGTCGCAGGACGCAGGGTTCGGGCCTAG | |
| | GGTAGGCTCTCCTGAATCGACAGGCGCCGGACCTCTGGTG | |
| | AGGGGAGGGATAAGTGAGGCGTCAGTTTCTCTGGTCGGTT | |
| | TTATGTACCTATCTTCTTAAGTAGCTGAAGCTCCGGTTTTG | |
| | AACTATGCGCTCGGGGTTGGCGAGTGTGTTTTGTGAAGTTT | |
| | TTTAGGCACCTTTTGAAATGTAATCATTTGGGTCAATATGT | |
| | AATTTTCAGTGTTAGACTAGTAAATTGTCCGCTAAATTCTG | |
| | GCCGTTTTTGGCTTTTTTGTTAG | |
| EGFP shRNA | TCAAGCTGACCCTGAAGTTCAT | 4 |
| (TXN-051, -059) | | |
| ZAP70 shRNA | CAGGCGTAGATCACCAGAATA | 5 |
| (TXN-077) | | |
| ZAP70 shRNA | CGCAACGTCCTGCTGGTTAAC | 6 |
| (TXN-078) | | |
| ZAP70 shRNA | GAACTTGGCTGCGGCAACTTT | 7 |
| (TXN-079) | | |
| LAT shRNA | CCTACTCTGTGTAATAGAATA,, | 8 |
| (TXN-080) | | |
| LAT shRNA | GGGCTCCAGATTACGAGAATC | 9 |
| (TXN-081) | | |
| LAT shRNA | GTCCATTGATGATTACGTGAA | 10 |
| (TXN-082) | | |
| ApoE shRNA | GCTGATGGACGAGACCATGAA | 11 |
| (ApoE_Ri-001) | | |
| ApoE shRNA | GAAGGAGTTGAAGGCCTACAA | 12 |
| (ApoE_Ri-002) | | |
| ApoE shRNA | TGCCCAGCGACAATCACTGAA | 13 |
| (ApoE_Ri-003) | | |
| ApoE shRNA | GACAATCACTGAACGCCGAAG | 14 |
| (ApoE_Ri-004) | | |
| HTT shRNA | TGGTTCAGTTACGGGTTAATT | 15 |
| (HTT-001) | | |
| HTT shRNA | TGTTGCCGCAGCATCACTAAT | 16 |
| (HTT-002) | | |
| HTT shRNA | GCACTCAAGAAGGACACAATA | 17 |
| (HTT-003) | | |
| TXN-036 | GATCTGGCCTCCGCGCCGGGTTTTGGCGCCTCCCGCGGGCG | 18 |
| (HubC short | CCCCCCTCCTCACGGCGAGCGCTGCCACGTCAGACGAAGG | |
| promoter-PEST- | GCGCAGGAGCGTCCTGATCCTTCCGCCCGGACGCTCAGGA | |
| tagged EGFP) | CAGCGGCCCGCTGCTCATAAGACTCGGCCTTAGAACCCCA | |
| | GTATCAGCAGAAGGACATTTTAGGACGGGACTTGGGTGAC | |
| | TCTAGGGCACTGGTTTTCTTTCCAGAGAGCGGAACAGGCG | |
| | AGGAAAAGTAGTCCCTTCTCGGCGATTCTGCGGAGGGATC | |
| | TCCGTGGGGCGGTGAACGCCGATGATTATATAAGGACGCG | |
| | CCGGGTGTGGCACAGCTCATATGGGCAAGCTGACCCTGAA | |
| | GTTCATCTGTCAAGAGGTCTACTTGAAGTCCCAGTCGAACG | |
| | GGAATTCGTTTCAAAAGTAGA | |
| TXN-037 | GGCCTCCGCGCCGGGTTTTGGCGCCTCCCGCGGGCGCCCCC | 19 |
| (UbiC promoter- | CTCCTCACGGCGAGCGCTGCCACGTCAGACGAAGGGCGCA | |
| PEST-tagged EGFP) | GCGAGCGTCCTGATCCTTCCGCCCGGACGCTCAGGACAGC | |
| | GGCCCGCTGCTCATAAGACTCGGCCTTAGAACCCCAGTATC | |
| | AGCAGAAGGACATTTTAGGACGGGACTTGGGTGACTCTAG | |
| | GGCACTGGTTTTCTTTCCAGAGAGCGGAACAGGCGAGGAA | |
| | AAGTAGTCCCTTCTCGGCGATTCTGCGGAGGGATCTCCGTG | |
| | GGGCGGTGAACGCCGATGATTATATAAGGACGCGCCGGGT | |
| | GTGGCACAGCTAGTTCCGTCGCAGCCGGGATTTGGGTCGC | |
| | GGTTCTTGTTTGTGGATCGCTGTGATCGTCACTTGGTCGCT | |
| | AGCGCTACCGGTCGCCACCATGTTGAGCAAGGGCGAGGAG | |
| | GACAACATGGCCATCATCAAGGAGTTCATGCGCTTCAAGG | |
| | TGCACATGGAGGGCTCCGTGAACGGCCACGAGTTCGAGAT | |
| | CGAGGGCGAGGGCGAGGGCCGCCCCTACGAGGGCACCCA | |
| | GACCGCCAAGCTGAAGGTGACCAAGGGCGGCCCCCTGCCC | |
| | TTCGCCTGGGACATCCTGTCCCCTCAGTTCATGTACGGCTC | |
| | CAAGGCCTACGTGAAGCACCCCGCCGACATCCCCGACTAC | |
| | TTGAAGCTGTCCTTCCCCGAGGGCTTCAAGTGGGAGCGCGT | |
| | GATGAACTTCGAGGACGGCGGCGTGGTGACCGTGACCCAG | |
| | GACTCCTCCCTGCAGGACGGCGAGTTCATCTACAAGGTGA | |
| | AGCTGCGCGGCACCAACTTCCCCTCCGACGGCCCCGTAATG | |
| | CAGAAGAAGACCATGGGCTGGGAGGCCTCCTCCGAGCGGA | |
| | TGTACCCCGAGGACGGCGCCCTGAAGGGCGAGATCAAGCA | |
| | GAGGCTGAAGCTGAAGGACGGCGGCCACTACGACGCCGAG | |
| | GTCAAGACCACCTACAAGGCCAAGAAGCCCGTGCAGCTGC | |
| | CCGGCGCCTACAACGTCAACATCAAGCTGGACATCACCTC | |
| | CCACAACGAGGACTACACCATCGTGGAACAGTACGAGCGC | |
| | GCCGAGGGCCGCCACTCCACCGGCGGCATGGACGAGCTGT | |
| | ACAAGTAATAGGAATTCAAGAAGGTATATTGCTGTTGACA | |
| | GTGAGCGTCAAGCTGACCCTGAAGTTCATTAGTGAAGCCA | |
| | CAGATGTAATGAACTTCAGGGTCAGCTTGCTGCCTACTGCC | |
| | TCGGACTTCAAGGG | |
| TXN-038 | CGGCCTCCGCGCCGGGTTTTGGCGCCTCCCGCGGGCGCCCC | 20 |
| (UbiC promoter plus | CCTCCTCACGGCGAGCGCTGCCACGTCAGACGAAGGGCGC | |
| miR-30-like hairpin- | AGCGAGCGTCCTGATCCTTCCGCCCGGACGCTCAGGACAG | |
| PEST-tagged EGFP) | CGGCCCGCTGCTCATAAGACTCGGCCTTAGAACCCCAGTAT | |
| | CAGCAGAAGGACATTTTAGGACGGGACTTGGGTGACTCTA | |
| | GGGCACTGGTTTTCTTTCCAGAGAGCGGAACAGGCGAGGA | |
| | AAAGTAGTCCCTTCTCGGCGATTCTGCGGAGGGATCTCCGT | |
| | GGGGCGGTGAACGCCGATGATTATATAAGGACGCGCCGGG | |
| | TGTGGCACAGCTAGTTCCGTCGCAGCCGGGATTTGGGTCGC | |
| | AGTTCTTGTTTGTGGATCGCTGTGATCGTCACTTGGTGAGT | |
| | AGCGGGCTGCTGGGCTGGCCGGGGCTTTCGTGGCCGCCGG | |
| | GCCGCTCGGTGGGACGGAGGCGTGTGGAGAGACCGCCAAG | |
| | GGCTGTAGTCTGGGTCCGCGAGCAAGGTTGCCCTGAACTG | |
| | GGGGTTGGGGGGAGCGCAGCAAAATGGCGGCTGTTCCCGA | |
| | GTCTTGAATGGAAGACGCTTGTGAGGCGGGCTGTGAGGTC | |
| | GTTGAAACAAGGTGGGGGGCATGGTGGGCGGCAAGAACCC | |
| | AAGGTCTTGAGGCCTTCGCTAATGCGGGAAAGCTCTTATTC | |
| | GGGTGAGATGGGCTGGGGCACCATCTGGGGACCCTGACGT | |
| | GAAGTTTGTCACTGACTGGAGAACTCGGTTTGTCGTCTGTT | |
| | GCGGGGGCGGCAGTTATGGCGGTGCCGTTGGGCAGTGCAC | |
| | CCGTACCTTTGGGAGCGCGCGCCCTCGTCGTGTCGTGACGT | |
| | CACCCGTTCTGTTTGCTGTTGACAGTGAGCGATCAAGCTGA | |
| | CCCTGAAGTTCATTAGTGAAGCCACAGATGTAATGAACTTC | |
| | AGGGTCAGCTTGCCTGCCTACTGCCTCGGACTTCAAGGGGG | |
| | CTTATAATGCAGGGTGGGGCCACCTGCCGGTAGGTGTGCG | |
| | GTAGGCTTTTCTCCGTCGCAGGACGCAGGGTTCGGGCCTAG | |
| | GGTAGGCTCTCCTGAATCGACAGGCGCCGGACCTCTGGTG | |
| | AGGGGAGGGATAAGTGAGGCGTCAGTTTCTCTGGTCGGTT | |
| | TTATGTACCTATCTTCTTAAGTAGCTGAAGCTCCGGTTTTG | |
| | AACTATGCGCTCGGGGTTGGCGAGTGTGTTTTGTGAAGTTT | |
| | TTTAGGCACCTTTTGAAATGTAATCATTTGGGTCAATATGT | |
| | AATTTTCAGTGTTAGACTAGTAAATTGTCCGCTAAATTCTG | |
| | GCCGTTTTTGGCTTTTTTGTTAG | |
| TXN-041 | ATGGATTATCCTGCCGCCAAGAGAGTGAAGCTGGACAGCA | 21 |
| (ZFHD1-hDHFR) | GAGAGAGGCCCTACGCCTGTCCTGTGGAAAGCTGCGACAG | |
| | ACGGTTCAGCAGAAGCGACGAGCTGACCCGGCACATCAGA | |
| | ATCCACACCGGCCAGAAACCTTTCCAGTGCCGGATCTGCAT | |
| | GCGGAACTTCAGCAGATCCGACCACCTGACCACACACATC | |
| | AGGACACACACAGGCGGAGGCAGACGGCGGAAGAAGAGA | |
| | ACCAGCATCGAGACAAACATCCGCGTGGCCCTGGAAAAGA | |
| | GCTTCCTGGAAAACCAGAAGCCTACCAGCGAAGAGATCAC | |
| | CATGATCGCCGACCAGCTGAACATGGAAAAAGAAGTGATC | |
| | CGCGTCTGGTTCTGCAACCGGCGGCAGAAAGAGAAGCGGA | |
| | TCAACGGCAGCCGGAAGAGATGTGCTGCTGGTGTTGGCGG | |
| | AGGACCTGCTGGATGTCCTGCTCCTGGAAGCACCCCTCTGA | |
| | AGAAGCCTAGAAGAGGCAGCCTGTCTGAGGCCCTGCTGCA | |
| | GCTCCAGTTCGACGATGAAGATCTGGGAGCCCTGCTGGGC | |
| | AACAGCACAGATCCTGCCGTGTTTACCGATCTGGCCAGCGT | |
| | GGACAACAGCGAGTTTCAGCAGCTCCTGAACCAGGGCATC | |
| | CCTGTGGCTCCTCACACCACAGAGCCCATGCTGATGGAATA | |
| | CCCCGAGGCCATCACCAGACTGGTCACCGGTGCTCAAAGA | |
| | CCTCCAGATCCAGCTCCTGCTCCACTGGGAGCACCTGGACT | |
| | GCCTAATGGACTGCTGTCTGGCGACGAGGACTTCAGCTCTA | |
| | TCGCCGACATGGATTTCAGCGCCCTGCTGAGCCAGATCAGC | |
| | TCTGGAGGTAGTGGTGGCGGATCCGGAATGGTGGGCTCTC | |
| | TGAATTGCATTGTGGCCGTGTCTCAGAACATGGGCATCGGC | |
| | AAGAACGGCGACCTTCCTTGGCCTCCTCTGCGGAACGAGCT | |
| | GCGGTACTTCCACAGAATGACCACCACCAGCAGCGTGGAA | |
| | GGCAAGCAGAACCTGGTCATCATGGGCAAGAAAACCTGGT | |
| | TCAGCATCGGCAGACCCCTGAAGGGCAGAATCAACCTGGT | |
| | GCTGAGCAGAGAGCTGAAAGAGCCTCCTCAGGGCGCCCAC | |
| | TTTCTGAGCAGATCTCTGGACGATGCCCTGAAGCTGACCGA | |
| | GCAGCCTGAACTGGCTAACAAGGTGGACATGGTCTGGATC | |
| | GTCGGCGGCAGCTCCGTGATCAAAGAAGCCATGAATCACC | |
| | CCGGCCACCTGAAACTGTTCGTGACCAGAATCATGCAGGA | |
| | CTTCGAGTCCGACACATTCTTCCCAGAGATCGACCTGGAAA | |
| | AGTACAAGCTGCTGCCAGAGTACCCCGGCGTGCTGTCCGA | |
| | TGTGCAAGAGGAAAAGGGCATCAAGTACAAGTTCGAGGTG | |
| | TACGAGAAGAACGATTAAGCTAGCGAACAGAGAAACAGG | |
| | AGAATATGGGCCAAACAGGATATCTGTGGTAAGCAGTTCC | |
| | TGCCCCGGCTCAGGGCCAAGAACAGTTGGAACAGCAGAAT | |
| | ATGGGCCAAACAGGATATCTGTGGTAAGCAGTTCCTGCCC | |
| | CGGCTCAGGGCCAAGAACAGATGGTCCCCAGATGCGGTCC | |
| | CGCCCTCAGCAGTTTCTAGAGAACCATCAGATGTTTCCAGG | |
| | GTGCCCCAAGGACCTGAAATGACCCTGTGCCTTATTTGAAC | |
| | TAACCAATCAGTTCGCTTCTCGCTTCTGTTCGCGCGCTTCTG | |
| | CTCCCCGAGCTCTATATAAGCAGAGCTCGTTTAGTGAACCG | |
| | TCAGATCGCTAGACTAGGCCACCATGAACCCAGCCATCAG | |
| | CGTCGCTCTCCTGCTCTCAGTCTTGCAGGTGTCCCGAGGGC | |
| | AGAAGGTGACCAGCCTGACAGCCTGCCTGGTGAACCAAAA | |
| | CCTTCGCCTGGACTGCCGCCATGAGAATAACACCAAGGAT | |
| | AACTCCATCCAGCATGAGTTCAGCCTGACCCGAGAGAAGA | |
| | GGAAGCACGTGCTCTCAGGCACCCTTGGGATACCCGAGCA | |
| | CACGTACCGCTCCCGCGTCACCCTCTCCAACCAGCCCTATA | |
| | TCAAGGTCCTTACCCTAGCCAACTTCACCACCAAGGATGAG | |
| | GGCGACTACTTTTGTGAGCTTCAAGTCTCGGGCGCGAATCC | |
| | CATGAGCTCCAATAAAAGTATCAGTGTGTATAGAGACAAG | |
| | CTGGTCAAGTGTGGCGGCATAAGCCTGCTGGTTCAGAACA | |
| | CATCCTGGATGCTGCTGCTGCTGCTTTCCCTCTCCCTCCTCC | |
| | AAGCCCTGGACTTCATTTCTCTG | |
| TXN-051 | TAATGATGGGCGCTCGAGTAATGATGGGCGGTCGACTAAT | 22 |
| (mCherry-shRNA | GATGGGCGCTCGAGTAATGATGGGCGTCTAGCTAATGATG | |
| targeting GFP) | GGCGCTCGAGTAATGATGGGCGGTCGACTAATGATGGGCG | |
| | CTCGAGTAATGATGGGCGTCTAGCTAATGATGGGCGCTCG | |
| | AGTAATGATGGGCGGTCGACTAATGATGGGCGCTCGAGTA | |
| | ATGATGGGCGTCTAGAAATAGGGGTTCAATATAAAAAGCC | |
| | GGCAGAGAGCTGTCCAAGTCCGCTAGCGCTACCGGTCGCC | |
| | ACCATGTTGAGCAAGGGCGAGGAGGACAACATGGCCATCA | |
| | TCAAGGAGTTCATGCGCTTCAAGGTGCACATGGAGGGCTC | |
| | CGTGAACGGCCACGAGTTCGAGATCGAGGGCGAGGGCGAG | |
| | GGCCGCCCCTACGAGGGCACCCAGACCGCCAAGCTGAAGG | |
| | TGACCAAGGGCGGCCCCCTGCCCTTCGCCTGGGACATCCTG | |
| | TCCCCTCAGTTCATGTACGGCTCCAAGGCCTACGTGAAGCA | |
| | CCCCGCCGACATCCCCGACTACTTGAAGCTGTCCTTCCCCG | |
| | AGGGCTTCAAGTGGGAGCGCGTGATGAACTTCGAGGACGG | |
| | CGGCGTGGTGACCGTGACCCAGGACTCCTCCCTGCAGGAC | |
| | GGCGAGTTCATCTACAAGGTGAAGCTGCGCGGCACCAACT | |
| | TCCCCTCCGACGGCCCCGTAATGCAGAAGAAGACCATGGG | |
| | CTGGGAGGCCTCCTCCGAGCGGATGTACCCCGAGGACGGC | |
| | GCCCTGAAGGGCGAGATCAAGCAGAGGCTGAAGCTGAAG | |
| | GACGGCGGCCACTACGACGCCGAGGTCAAGACCACCTACA | |
| | AGGCCAAGAAGCCCGTGCAGCTGCCCGGCGCCTACAACGT | |
| | CAACATCAAGCTGGACATCACCTCCCACAACGAGGACTAC | |
| | ACCATCGTGGAACAGTACGAGCGCGCCGAGGGCCGCCACT | |
| | CCACCGGCGGCATGGACGAGCTGTACAAGTAATAGGAATT | |
| | CAAGAAGGTATATTGCTGTTGACAGTGAGCGTCAAGCTGA | |
| | CCCTGAAGTTCATTAGTGAAGCCACAGATGTAATGAACTTC | |
| | AGGGTCAGCTTGCTGCCTACTGCCTCGGACTTCAAGGG | |
| ApoE_Ri-004 | TAATGATGGGCGCACGAGTAATGATGGGCGGACGACTAAT | 23 |
| (mCherry-shRNA | GATGGGCGCACGAGTAATGATGGGCGTCTAGCTAATGATG | |
| targeting ApoE) | GGCGCTAGAGTAATGATGGGCGGTAGACTAATGATGGGCG | |
| | CTCCAGTAATGATGGGCGTTCTAGCTCTAGAGGGTATATAA | |
| | TGGGGGCCACGCTAGCGCTACCGGTCGCCACCATGTTGAG | |
| | CAAGGGCGAGGAGGACAACATGGCCATCATCAAGGAGTTC | |
| | ATGCGCTTCAAGGTGCACATGGAGGGCTCCGTGAACGGCC | |
| | ACGAGTTCGAGATCGAGGGCGAGGGCGAGGGCCGCCCCTA | |
| | CGAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGGACAATCACTGAACGCCGAA | |
| | GTAGTGAAGCCACAGATGTACTTCGGCGTTCAGTGATTGTC | |
| | TGCCTACTGCCTCGGACTTCAAGGG | |
| HTT-001 | TAATGATGGGCGCTCGAGTAATGATGGGCGGTCGACTAAT | 24 |
| (mCherry-shRNA | GATGGGCGCTCGAGTAATGATGGGCGTCTAGCTAATGATG | |
| targeting HTT) | GGCGCTCGAGTAATGATGGGCGGTCGACTAATGATGGGCG | |
| | CTCGAGTAATGATGGGCGTCTAGCTAATGATGGGCGCTCG | |
| | AGTAATGATGGGCGGTCGACTAATGATGGGCGCTCGAGTA | |
| | ATGATGGGCGTCTAGAAATAGGGGTTCAATATAAAAAGCC | |
| | GGCAGAGAGCTGTCCAAGTCCGCTAGCGCTACCGGTCGCC | |
| | ACCATGTTGAGCAAGGGCGAGGAGGACAACATGGCCATCA | |
| | TCAAGGAGTTCATGCGCTTCAAGGTGCACATGGAGGGCTC | |
| | CGTGAACGGCCACGAGTTCGAGATCGAGGGCGAGGGCGAG | |
| | GGCCGCCCCTACGAGGGCACCCAGACCGCCAAGCTGAAGG | |
| | TGACCAAGGGCGGCCCCCTGCCCTTCGCCTGGGACATCCTG | |
| | TCCCCTCAGTTCATGTACGGCTCCAAGGCCTACGTGAAGCA | |
| | CCCCGCCGACATCCCCGACTACTTGAAGCTGTCCTTCCCCG | |
| | AGGGCTTCAAGTGGGAGCGCGTGATGAACTTCGAGGACGG | |
| | CGGCGTGGTGACCGTGACCCAGGACTCCTCCCTGCAGGAC | |
| | GGCGAGTTCATCTACAAGGTGAAGCTGCGCGGCACCAACT | |
| | TCCCCTCCGACGGCCCCGTAATGCAGAAGAAGACCATGGG | |
| | CTGGGAGGCCTCCTCCGAGCGGATGTACCCCGAGGACGGC | |
| | GCCCTGAAGGGCGAGATCAAGCAGAGGCTGAAGCTGAAG | |
| | GACGGCGGCCACTACGACGCCGAGGTCAAGACCACCTACA | |
| | AGGCCAAGAAGCCCGTGCAGCTGCCCGGCGCCTACAACGT | |
| | CAACATCAAGCTGGACATCACCTCCCACAACGAGGACTAC | |
| | ACCATCGTGGAACAGTACGAGCGCGCCGAGGGCCGCCACT | |
| | CCACCGGCGGCATGGACGAGCTGTACAAGTAATAGGAATT | |
| | CAAGAAGGTATATTGCTGTTGACAGTGAGCGTGGTTCAGTT | |
| | ACGGGTTAATTTAGTGAAGCCACAGATGTAAATTAACCCG | |
| | TAACTGAACCATGCCTACTGCCTCGGACTTCAAGGG | |
| TXN-059 | TAATGATGGGCGCTCGAGTAATGATGGGCGGTCGACTAAT | 25 |
| (12xZFHD1 YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| GFP-EFS-ZFHD1- | AGTAATGATGGGCGGTCGACTAATGATGGGCGCTCGAGTA | |
| hDHFR-MND-Thy | ATGATGGGCGTCTAGAATCTAGAGGGTATATAATGGGGGC | |
| 1.2) | CAACCGGTCCTGCAGCGGAGGATCCGCCACCATGTTGAGC | |
| | AAGGGCGAGGAGGACAACATGGCCATCATCAAGGAGTTCA | |
| | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGTCAAGCTGACCCTGAAGTTCA | |
| | TTAGTGAAGCCACAGATGTAATGAACTTCAGGGTCAGCTT | |
| | GCTGCCTACTGCCTCGGACTTCAAGGGACGCGTTGGCTTAC | |
| | CTCCCATTGACCTTATGTACTGGGCAAAACCCATTGGAAAG | |
| | TCCCTATTGACTCAATGTACTTGGCTCCAATGGGACTTTCC | |
| | TGTTGACTCACCCCCTATTGACCTTATGTACTGGGCAAAAC | |
| | CCAATGGAAAGTCCCTATTGAGTCAGTGTACTTGGCTCCAA | |
| | AGGGTTTTTCCCATTGACTAGTCGAGGAAGAGCATGCGTG | |
| | AGGCTCCGGTGCCCGTCAGTGGGCAGAGCGCACATCGCCC | |
| | ACAGTCCCCGAGAAGTTGGGGGGAGGGGTCGGCAATTGAA | |
| | CCGGTGCCTAGAGAAGGTGGCGCGGGGTAAACTGGGAAAG | |
| | TGATGTCGTGTACTGGCTCCGCCTTTTTCCCGAGGGTGGGG | |
| | GAGAACCGTATATAAGTGCAGTAGTCGCCGTGAACGTTCTT | |
| | TTTCGCAACGGGTTTGCCGCCAGAACACAGGCTAGACTAG | |
| | TGCCACCATGGATTATCCTGCCGCCAAGAGAGTGAAGCTG | |
| | GACAGCAGAGAGAGGCCCTACGCCTGTCCTGTGGAAAGCT | |
| | GCGACAGACGGTTCAGCAGAAGCGACGAGCTGACCCGGCA | |
| | CATCAGAATCCACACCGGCCAGAAACCTTTCCAGTGCCGG | |
| | ATCTGCATGCGGAACTTCAGCAGATCCGACCACCTGACCA | |
| | CACACATCAGGACACACACAGGCGGAGGCAGACGGCGGA | |
| | AGAAGAGAACCAGCATCGAGACAAACATCCGCGTGGCCCT | |
| | GGAAAAGAGCTTCCTGGAAAACCAGAAGCCTACCAGCGAA | |
| | GAGATCACCATGATCGCCGACCAGCTGAACATGGAAAAAG | |
| | AAGTGATCCGCGTCTGGTTCTGCAACCGGCGGCAGAAAGA | |
| | GAAGCGGATCAACGGCAGCCGGAAGAGATGTGCTGCTGGT | |
| | GTTGGCGGAGGACCTGCTGGATGTCCTGCTCCTGGAAGCA | |
| | CCCCTCTGAAGAAGCCTAGAAGAGGCAGCCTGTCTGAGGC | |
| | CCTGCTGCAGCTCCAGTTCGACGATGAAGATCTGGGAGCC | |
| | CTGCTGGGCAACAGCACAGATCCTGCCGTGTTTACCGATCT | |
| | GGCCAGCGTGGACAACAGCGAGTTTCAGCAGCTCCTGAAC | |
| | CAGGGCATCCCTGTGGCTCCTCACACCACAGAGCCCATGCT | |
| | GATGGAATACCCCGAGGCCATCACCAGACTGGTCACCGGT | |
| | GCTCAAAGACCTCCAGATCCAGCTCCTGCTCCACTGGGAGC | |
| | ACCTGGACTGCCTAATGGACTGCTGTCTGGCGACGAGGAC | |
| | TTCAGCTCTATCGCCGACATGGATTTCAGCGCCCTGCTGAG | |
| | CCAGATCAGCTCTGGAGGTAGTGGTGGCGGATCCGGAATG | |
| | GTGGGCTCTCTGAATTGCATTGTGGCCGTGTCTCAGAACAT | |
| | GGGCATCGGCAAGAACGGCGACCTTCCTTGGCCTCCTCTGC | |
| | GGAACGAGCTGCGGTACTTCCACAGAATGACCACCACCAG | |
| | CAGCGTGGAAGGCAAGCAGAACCTGGTCATCATGGGCAAG | |
| | AAAACCTGGTTCAGCATCGGCAGACCCCTGAAGGGCAGAA | |
| | TCAACCTGGTGCTGAGCAGAGAGCTGAAAGAGCCTCCTCA | |
| | GGGCGCCCACTTTCTGAGCAGATCTCTGGACGATGCCCTGA | |
| | AGCTGACCGAGCAGCCTGAACTGGCTAACAAGGTGGACAT | |
| | GGTCTGGATCGTCGGCGGCAGCTCCGTGATCAAAGAAGCC | |
| | ATGAATCACCCCGGCCACCTGAAACTGTTCGTGACCAGAA | |
| | TCATGCAGGACTTCGAGTCCGACACATTCTTCCCAGAGATC | |
| | GACCTGGAAAAGTACAAGCTGCTGCCAGAGTACCCCGGCG | |
| | TGCTGTCCGATGTGCAAGAGGAAAAGGGCATCAAGTACAA | |
| | GTTCGAGGTGTACGAGAAGAACGATTAAGCTAGCGAACAG | |
| | AGAAACAGGAGAATATGGGCCAAACAGGATATCTGTGGTA | |
| | AGCAGTTCCTGCCCCGGCTCAGGGCCAAGAACAGTTGGAA | |
| | CAGCAGAATATGGGCCAAACAGGATATCTGTGGTAAGCAG | |
| | TTCCTGCCCCGGCTCAGGGCCAAGAACAGATGGTCCCCAG | |
| | ATGCGGTCCCGCCCTCAGCAGTTTCTAGAGAACCATCAGAT | |
| | GTTTCCAGGGTGCCCCAAGGACCTGAAATGACCCTGTGCCT | |
| | TATTTGAACTAACCAATCAGTTCGCTTCTCGCTTCTGTTCGC | |
| | GCGCTTCTGCTCCCCGAGCTCTATATAAGCAGAGCTCGTTT | |
| | AGTGAACCGTCAGATCGCTAGACTAGGCCACCATGAACCC | |
| | AGCCATCAGCGTCGCTCTCCTGCTCTCAGTCTTGCAGGTGT | |
| | CCCGAGGGCAGAAGGTGACCAGCCTGACAGCCTGCCTGGT | |
| | GAACCAAAACCTTCGCCTGGACTGCCGCCATGAGAATAAC | |
| | ACCAAGGATAACTCCATCCAGCATGAGTTCAGCCTGACCC | |
| | GAGAGAAGAGGAAGCACGTGCTCTCAGGCACCCTTGGGAT | |
| | ACCCGAGCACACGTACCGCTCCCGCGTCACCCTCTCCAACC | |
| | AGCCCTATATCAAGGTCCTTACCCTAGCCAACTTCACCACC | |
| | AAGGATGAGGGCGACTACTTTTGTGAGCTTCAAGTCTCGG | |
| | GCGCGAATCCCATGAGCTCCAATAAAAGTATCAGTGTGTA | |
| | TAGAGACAAGCTGGTCAAGTGTGGCGGCATAAGCCTGCTG | |
| | GTTCAGAACACATCCTGGATGCTGCTGCTGCTGCTTTCCCT | |
| | CTCCCTCCTCCAAGCCCTGGACTTCATTTCTCTG | |
| TXN-077 | TAATGATGGGCGCTCGAGTAATGATGGGCGGTCGACTAAT | 26 |
| (12xZFHD1 YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| ZAP70-EFS- | AGTAATGATGGGCGGTCGACTAATGATGGGCGCTCGAGTA | |
| ZFHD1-hDHFR- | ATGATGGGCGTCTAGAATCTAGAGGGTATATAATGGGGGC | |
| MND-CD19 CAR) | CAACCGGTCCTGCAGCGGAGGATCCGCCACCATGTTGAGC | |
| | AAGGGCGAGGAGGACAACATGGCCATCATCAAGGAGTTCA | |
| | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGCAGGCGTAGATCACCAGAAT | |
| | ATAGTGAAGCCACAGATGTATATTCTGGTGATCTACGCCTG | |
| | TGCCTACTGCCTCGGACTTCAAGGGACGCGTTGGCTTACCT | |
| | CCCATTGACCTTATGTACTGGGCAAAACCCATTGGAAAGTC | |
| | CCTATTGACTCAATGTACTTGGCTCCAATGGGACTTTCCTG | |
| | TTGACTCACCCCCTATTGACCTTATGTACTGGGCAAAACCC | |
| | AATGGAAAGTCCCTATTGAGTCAGTGTACTTGGCTCCAAAG | |
| | GGTTTTTCCCATTGACTAGTCGAGGAAGAGCATGCGTGAG | |
| | GCTCCGGTGCCCGTCAGTGGGCAGAGCGCACATCGCCCAC | |
| | AGTCCCCGAGAAGTTGGGGGGAGGGGTCGGCAATTGAACC | |
| | GGTGCCTAGAGAAGGTGGCGCGGGGTAAACTGGGAAAGTG | |
| | ATGTCGTGTACTGGCTCCGCCTTTTTCCCGAGGGTGGGGGA | |
| | GAACCGTATATAAGTGCAGTAGTCGCCGTGAACGTTCTTTT | |
| | TCGCAACGGGTTTGCCGCCAGAACACAGGCTAGACTAGTG | |
| | CCACCATGGATTATCCTGCCGCCAAGAGAGTGAAGCTGGA | |
| | CAGCAGAGAGAGGCCCTACGCCTGTCCTGTGGAAAGCTGC | |
| | GACAGACGGTTCAGCAGAAGCGACGAGCTGACCCGGCACA | |
| | TCAGAATCCACACCGGCCAGAAACCTTTCCAGTGCCGGAT | |
| | CTGCATGCGGAACTTCAGCAGATCCGACCACCTGACCACA | |
| | CACATCAGGACACACACAGGCGGAGGCAGACGGCGGAAG | |
| | AAGAGAACCAGCATCGAGACAAACATCCGCGTGGCCCTGG | |
| | AAAAGAGCTTCCTGGAAAACCAGAAGCCTACCAGCGAAGA | |
| | GATCACCATGATCGCCGACCAGCTGAACATGGAAAAAGAA | |
| | GTGATCCGCGTCTGGTTCTGCAACCGGCGGCAGAAAGAGA | |
| | AGCGGATCAACGGCAGCCGGAAGAGATGTGCTGCTGGTGT | |
| | TGGCGGAGGACCTGCTGGATGTCCTGCTCCTGGAAGCACC | |
| | CCTCTGAAGAAGCCTAGAAGAGGCAGCCTGTCTGAGGCCC | |
| | TGCTGCAGCTCCAGTTCGACGATGAAGATCTGGGAGCCCT | |
| | GCTGGGCAACAGCACAGATCCTGCCGTGTTTACCGATCTGG | |
| | CCAGCGTGGACAACAGCGAGTTTCAGCAGCTCCTGAACCA | |
| | GGGCATCCCTGTGGCTCCTCACACCACAGAGCCCATGCTGA | |
| | TGGAATACCCCGAGGCCATCACCAGACTGGTCACCGGTGC | |
| | TCAAAGACCTCCAGATCCAGCTCCTGCTCCACTGGGAGCAC | |
| | CTGGACTGCCTAATGGACTGCTGTCTGGCGACGAGGACTTC | |
| | AGCTCTATCGCCGACATGGATTTCAGCGCCCTGCTGAGCCA | |
| | GATCAGCTCTGGAGGTAGTGGTGGCGGATCCGGAATGGTG | |
| | GGCTCTCTGAATTGCATTGTGGCCGTGTCTCAGAACATGGG | |
| | CATCGGCAAGAACGGCGACCTTCCTTGGCCTCCTCTGCGGA | |
| | ACGAGCTGCGGTACTTCCACAGAATGACCACCACCAGCAG | |
| | CGTGGAAGGCAAGCAGAACCTGGTCATCATGGGCAAGAAA | |
| | ACCTGGTTCAGCATCGGCAGACCCCTGAAGGGCAGAATCA | |
| | ACCTGGTGCTGAGCAGAGAGCTGAAAGAGCCTCCTCAGGG | |
| | CGCCCACTTTCTGAGCAGATCTCTGGACGATGCCCTGAAGC | |
| | TGACCGAGCAGCCTGAACTGGCTAACAAGGTGGACATGGT | |
| | CTGGATCGTCGGCGGCAGCTCCGTGATCAAAGAAGCCATG | |
| | AATCACCCCGGCCACCTGAAACTGTTCGTGACCAGAATCAT | |
| | GCAGGACTTCGAGTCCGACACATTCTTCCCAGAGATCGACC | |
| | TGGAAAAGTACAAGCTGCTGCCAGAGTACCCCGGCGTGCT | |
| | GTCCGATGTGCAAGAGGAAAAGGGCATCAAGTACAAGTTC | |
| | GAGGTGTACGAGAAGAACGATTAAGCTAGCGAACAGAGA | |
| | AACAGGAGAATATGGGCCAAACAGGATATCTGTGGTAAGC | |
| | AGTTCCTGCCCCGGCTCAGGGCCAAGAACAGTTGGAACAG | |
| | CAGAATATGGGCCAAACAGGATATCTGTGGTAAGCAGTTC | |
| | CTGCCCCGGCTCAGGGCCAAGAACAGATGGTCCCCAGATG | |
| | CGGTCCCGCCCTCAGCAGTTTCTAGAGAACCATCAGATGTT | |
| | TCCAGGGTGCCCCAAGGACCTGAAATGACCCTGTGCCTTAT | |
| | TTGAACTAACCAATCAGTTCGCTTCTCGCTTCTGTTCGCGC | |
| | GCTTCTGCTCCCCGAGCTCTATATAAGCAGAGCTCGTTTAG | |
| | TGAACCGTCAGATCGCCACCACTAGTGCCACCATGGCTCTG | |
| | CCTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |
| TXN-078 | TAATGATGGGCGCTCGAGTAATGATGGGCGGTCGACTAAT | 27 |
| (12xZFHD1 YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| ZAP70-EFS- | AGTAATGATGGGCGGTCGACTAATGATGGGCGCTCGAGTA | |
| ZFHD1-hDHFR- | ATGATGGGCGTCTAGAATCTAGAGGGTATATAATGGGGGC | |
| MND-CD19 CAR) | CAACCGGTCCTGCAGCGGAGGATCCGCCACCATGTTGAGC | |
| | AAGGGCGAGGAGGACAACATGGCCATCATCAAGGAGTTCA | |
| | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGCGCAACGTCCTGCTGGTTAAC | |
| | TAGTGAAGCCACAGATGTAGTTAACCAGCAGGACGTTGCG | |
| | TGCCTACTGCCTCGGACTTCAAGGGACGCGTTGGCTTACCT | |
| | CCCATTGACCTTATGTACTGGGCAAAACCCATTGGAAAGTC | |
| | CCTATTGACTCAATGTACTTGGCTCCAATGGGACTTTCCTG | |
| | TTGACTCACCCCCTATTGACCTTATGTACTGGGCAAAACCC | |
| | AATGGAAAGTCCCTATTGAGTCAGTGTACTTGGCTCCAAAG | |
| | GGTTTTTCCCATTGACTAGTCGAGGAAGAGCATGCGTGAG | |
| | GCTCCGGTGCCCGTCAGTGGGCAGAGCGCACATCGCCCAC | |
| | AGTCCCCGAGAAGTTGGGGGGAGGGGTCGGCAATTGAACC | |
| | GGTGCCTAGAGAAGGTGGCGCGGGGTAAACTGGGAAAGTG | |
| | ATGTCGTGTACTGGCTCCGCCTTTTTCCCGAGGGTGGGGGA | |
| | GAACCGTATATAAGTGCAGTAGTCGCCGTGAACGTTCTTTT | |
| | TCGCAACGGGTTTGCCGCCAGAACACAGGCTAGACTAGTG | |
| | CCACCATGGATTATCCTGCCGCCAAGAGAGTGAAGCTGGA | |
| | CAGCAGAGAGAGGCCCTACGCCTGTCCTGTGGAAAGCTGC | |
| | GACAGACGGTTCAGCAGAAGCGACGAGCTGACCCGGCACA | |
| | TCAGAATCCACACCGGCCAGAAACCTTTCCAGTGCCGGAT | |
| | CTGCATGCGGAACTTCAGCAGATCCGACCACCTGACCACA | |
| | CACATCAGGACACACACAGGCGGAGGCAGACGGCGGAAG | |
| | AAGAGAACCAGCATCGAGACAAACATCCGCGTGGCCCTGG | |
| | AAAAGAGCTTCCTGGAAAACCAGAAGCCTACCAGCGAAGA | |
| | GATCACCATGATCGCCGACCAGCTGAACATGGAAAAAGAA | |
| | GTGATCCGCGTCTGGTTCTGCAACCGGCGGCAGAAAGAGA | |
| | AGCGGATCAACGGCAGCCGGAAGAGATGTGCTGCTGGTGT | |
| | TGGCGGAGGACCTGCTGGATGTCCTGCTCCTGGAAGCACC | |
| | CCTCTGAAGAAGCCTAGAAGAGGCAGCCTGTCTGAGGCCC | |
| | TGCTGCAGCTCCAGTTCGACGATGAAGATCTGGGAGCCCT | |
| | GCTGGGCAACAGCACAGATCCTGCCGTGTTTACCGATCTGG | |
| | CCAGCGTGGACAACAGCGAGTTTCAGCAGCTCCTGAACCA | |
| | GGGCATCCCTGTGGCTCCTCACACCACAGAGCCCATGCTGA | |
| | TGGAATACCCCGAGGCCATCACCAGACTGGTCACCGGTGC | |
| | TCAAAGACCTCCAGATCCAGCTCCTGCTCCACTGGGAGCAC | |
| | CTGGACTGCCTAATGGACTGCTGTCTGGCGACGAGGACTTC | |
| | AGCTCTATCGCCGACATGGATTTCAGCGCCCTGCTGAGCCA | |
| | GATCAGCTCTGGAGGTAGTGGTGGCGGATCCGGAATGGTG | |
| | GGCTCTCTGAATTGCATTGTGGCCGTGTCTCAGAACATGGG | |
| | CATCGGCAAGAACGGCGACCTTCCTTGGCCTCCTCTGCGGA | |
| | ACGAGCTGCGGTACTTCCACAGAATGACCACCACCAGCAG | |
| | CGTGGAAGGCAAGCAGAACCTGGTCATCATGGGCAAGAAA | |
| | ACCTGGTTCAGCATCGGCAGACCCCTGAAGGGCAGAATCA | |
| | ACCTGGTGCTGAGCAGAGAGCTGAAAGAGCCTCCTCAGGG | |
| | CGCCCACTTTCTGAGCAGATCTCTGGACGATGCCCTGAAGC | |
| | TGACCGAGCAGCCTGAACTGGCTAACAAGGTGGACATGGT | |
| | CTGGATCGTCGGCGGCAGCTCCGTGATCAAAGAAGCCATG | |
| | AATCACCCCGGCCACCTGAAACTGTTCGTGACCAGAATCAT | |
| | GCAGGACTTCGAGTCCGACACATTCTTCCCAGAGATCGACC | |
| | TGGAAAAGTACAAGCTGCTGCCAGAGTACCCCGGCGTGCT | |
| | GTCCGATGTGCAAGAGGAAAAGGGCATCAAGTACAAGTTC | |
| | GAGGTGTACGAGAAGAACGATTAAGCTAGCGAACAGAGA | |
| | AACAGGAGAATATGGGCCAAACAGGATATCTGTGGTAAGC | |
| | AGTTCCTGCCCCGGCTCAGGGCCAAGAACAGTTGGAACAG | |
| | CAGAATATGGGCCAAACAGGATATCTGTGGTAAGCAGTTC | |
| | CTGCCCCGGCTCAGGGCCAAGAACAGATGGTCCCCAGATG | |
| | CGGTCCCGCCCTCAGCAGTTTCTAGAGAACCATCAGATGTT | |
| | TCCAGGGTGCCCCAAGGACCTGAAATGACCCTGTGCCTTAT | |
| | TTGAACTAACCAATCAGTTCGCTTCTCGCTTCTGTTCGCGC | |
| | GCTTCTGCTCCCCGAGCTCTATATAAGCAGAGCTCGTTTAG | |
| | TGAACCGTCAGATCGCCACCACTAGTGCCACCATGGCTCTG | |
| | CCTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |
| TXN-079 | TAATGATGGGCGCTCGAGTAATGATGGGCGGTCGACTAAT | 28 |
| (12xZFHD1 YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| ZAP70-EFS- | AGTAATGATGGGCGGTCGACTAATGATGGGCGCTCGAGTA | |
| ZFHD1-hDHFR- | ATGATGGGCGTCTAGAATCTAGAGGGTATATAATGGGGGC | |
| MND-CD19 CAR) | CAACCGGTCCTGCAGCGGAGGATCCGCCACCATGTTGAGC | |
| | AAGGGCGAGGAGGACAACATGGCCATCATCAAGGAGTTCA | |
| | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGGAACTTGGCTGCGGCAACTTT | |
| | TAGTGAAGCCACAGATGTAAAAGTTGCCGCAGCCAAGTTC | |
| | TGCCTACTGCCTCGGACTTCAAGGGACGCGTTGGCTTACCT | |
| | CCCATTGACCTTATGTACTGGGCAAAACCCATTGGAAAGTC | |
| | CCTATTGACTCAATGTACTTGGCTCCAATGGGACTTTCCTG | |
| | TTGACTCACCCCCTATTGACCTTATGTACTGGGCAAAACCC | |
| | AATGGAAAGTCCCTATTGAGTCAGTGTACTTGGCTCCAAAG | |
| | GGTTTTTCCCATTGACTAGTCGAGGAAGAGCATGCGTGAG | |
| | GCTCCGGTGCCCGTCAGTGGGCAGAGCGCACATCGCCCAC | |
| | AGTCCCCGAGAAGTTGGGGGGAGGGGTCGGCAATTGAACC | |
| | GGTGCCTAGAGAAGGTGGCGCGGGGTAAACTGGGAAAGTG | |
| | ATGTCGTGTACTGGCTCCGCCTTTTTCCCGAGGGTGGGGGA | |
| | GAACCGTATATAAGTGCAGTAGTCGCCGTGAACGTTCTTTT | |
| | TCGCAACGGGTTTGCCGCCAGAACACAGGCTAGACTAGTG | |
| | CCACCATGGATTATCCTGCCGCCAAGAGAGTGAAGCTGGA | |
| | CAGCAGAGAGAGGCCCTACGCCTGTCCTGTGGAAAGCTGC | |
| | GACAGACGGTTCAGCAGAAGCGACGAGCTGACCCGGCACA | |
| | TCAGAATCCACACCGGCCAGAAACCTTTCCAGTGCCGGAT | |
| | CTGCATGCGGAACTTCAGCAGATCCGACCACCTGACCACA | |
| | CACATCAGGACACACACAGGCGGAGGCAGACGGCGGAAG | |
| | AAGAGAACCAGCATCGAGACAAACATCCGCGTGGCCCTGG | |
| | AAAAGAGCTTCCTGGAAAACCAGAAGCCTACCAGCGAAGA | |
| | GATCACCATGATCGCCGACCAGCTGAACATGGAAAAAGAA | |
| | GTGATCCGCGTCTGGTTCTGCAACCGGCGGCAGAAAGAGA | |
| | AGCGGATCAACGGCAGCCGGAAGAGATGTGCTGCTGGTGT | |
| | TGGCGGAGGACCTGCTGGATGTCCTGCTCCTGGAAGCACC | |
| | CCTCTGAAGAAGCCTAGAAGAGGCAGCCTGTCTGAGGCCC | |
| | TGCTGCAGCTCCAGTTCGACGATGAAGATCTGGGAGCCCT | |
| | GCTGGGCAACAGCACAGATCCTGCCGTGTTTACCGATCTGG | |
| | CCAGCGTGGACAACAGCGAGTTTCAGCAGCTCCTGAACCA | |
| | GGGCATCCCTGTGGCTCCTCACACCACAGAGCCCATGCTGA | |
| | TGGAATACCCCGAGGCCATCACCAGACTGGTCACCGGTGC | |
| | TCAAAGACCTCCAGATCCAGCTCCTGCTCCACTGGGAGCAC | |
| | CTGGACTGCCTAATGGACTGCTGTCTGGCGACGAGGACTTC | |
| | AGCTCTATCGCCGACATGGATTTCAGCGCCCTGCTGAGCCA | |
| | GATCAGCTCTGGAGGTAGTGGTGGCGGATCCGGAATGGTG | |
| | GGCTCTCTGAATTGCATTGTGGCCGTGTCTCAGAACATGGG | |
| | CATCGGCAAGAACGGCGACCTTCCTTGGCCTCCTCTGCGGA | |
| | ACGAGCTGCGGTACTTCCACAGAATGACCACCACCAGCAG | |
| | CGTGGAAGGCAAGCAGAACCTGGTCATCATGGGCAAGAAA | |
| | ACCTGGTTCAGCATCGGCAGACCCCTGAAGGGCAGAATCA | |
| | ACCTGGTGCTGAGCAGAGAGCTGAAAGAGCCTCCTCAGGG | |
| | CGCCCACTTTCTGAGCAGATCTCTGGACGATGCCCTGAAGC | |
| | TGACCGAGCAGCCTGAACTGGCTAACAAGGTGGACATGGT | |
| | CTGGATCGTCGGCGGCAGCTCCGTGATCAAAGAAGCCATG | |
| | AATCACCCCGGCCACCTGAAACTGTTCGTGACCAGAATCAT | |
| | GCAGGACTTCGAGTCCGACACATTCTTCCCAGAGATCGACC | |
| | TGGAAAAGTACAAGCTGCTGCCAGAGTACCCCGGCGTGCT | |
| | GTCCGATGTGCAAGAGGAAAAGGGCATCAAGTACAAGTTC | |
| | GAGGTGTACGAGAAGAACGATTAAGCTAGCGAACAGAGA | |
| | AACAGGAGAATATGGGCCAAACAGGATATCTGTGGTAAGC | |
| | AGTTCCTGCCCCGGCTCAGGGCCAAGAACAGTTGGAACAG | |
| | CAGAATATGGGCCAAACAGGATATCTGTGGTAAGCAGTTC | |
| | CTGCCCCGGCTCAGGGCCAAGAACAGATGGTCCCCAGATG | |
| | CGGTCCCGCCCTCAGCAGTTTCTAGAGAACCATCAGATGTT | |
| | TCCAGGGTGCCCCAAGGACCTGAAATGACCCTGTGCCTTAT | |
| | TTGAACTAACCAATCAGTTCGCTTCTCGCTTCTGTTCGCGC | |
| | GCTTCTGCTCCCCGAGCTCTATATAAGCAGAGCTCGTTTAG | |
| | TGAACCGTCAGATCGCCACCACTAGTGCCACCATGGCTCTG | |
| | CCTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |
| TCR-004 (EF1a- | CGTGAGGCTCCGGTGCCCGTCAGTGGGCAGAGCGCACATC | 29 |
| TCR control C128) | GCCCACAGTCCCCGAGAAGTTGGGGGGAGGGGTCGGCAAT | |
| | TGAACCGGTGCCTAGAGAAGGTGGCGCGGGGTAAACTGGG | |
| | AAAGTGATGTCGTGTACTGGCTCCGCCTTTTTCCCGAGGGT | |
| | GGGGGAGAACCGTATATAAGTGCAGTAGTCGCCGTGAACG | |
| | TTCTTTTTCGCAACGGGTTTGCCGCCAGAACACAGGTAAGT | |
| | GCCGTGTGTGGTTCCCGCGGGCCTGGCCTCTTTACGGGTTA | |
| | TGGCCCTTGCGTGCCTTGAATTACTTCCACCTGGCTGCAGT | |
| | ACGTGATTCTTGATCCCGAGCTTCGGGTTGGAAGTGGGTGG | |
| | GAGAGTTCGAGGCCTTGCGCTTAAGGAGCCCCTTCGCCTCG | |
| | TGCTTGAGTTGAGGCCTGGCCTGGGCGCTGGGGCCGCCGC | |
| | GTGCGAATCTGGTGGCACCTTCGCGCCTGTCTCGCTGCTTT | |
| | CGATAAGTCTCTAGCCATTTAAAATTTTTGATGACCTGCTG | |
| | CGACGCTTTTTTTCTGGCAAGATAGTCTTGTAAATGCGGGC | |
| | CAAGATCTGCACACTGGTATTTCGGTTTTTGGGGCCGCGGG | |
| | CGGCGACGGGGCCCGTGCGTCCCAGCGCACATGTTCGGCG | |
| | AGGCGGGGCCTGCGAGCGCGGCCACCGAGAATCGGACGG | |
| | GGGTAGTCTCAAGCTGGCCGGCCTGCTCTGGTGCCTGGCCT | |
| | CGCGCCGCCGTGTATCGCCCCGCCCTGGGCGGCAAGGCTG | |
| | GCCCGGTCGGCACCAGTTGCGTGAGCGGAAAGATGGCCGC | |
| | TTCCCGGCCCTGCTGCAGGGAGCTCAAAATGGAGGACGCG | |
| | GCGCTCGGGAGAGCGGGCGGGTGAGTCACCCACACAAAGG | |
| | AAAAGGGCCTTTCCGTCCTCAGCCGTCGCTTCATGTGACTC | |
| | CACTGAGTACCGGGCGCCGTCCAGGCACCTCGATTAGTTCT | |
| | CGTGCTTTTGGAGTACGTCGTCTTTAGGTTGGGGGGAGGGG | |
| | TTTTATGCGATGGAGTTTCCCCACACTGAGTGGGTGGAGAC | |
| | TGAAGTTAGGCCAGCTTGGCACTTGATGTAATTCTCCTTGG | |
| | AATTTGCCCTTTTTGAGTTTGGATCTTGGTTCATTCTCAAGC | |
| | CTCAGACAGTGGTTCAAAGTTTTTTTCTTCCATTTCAGGTGT | |
| | CGTGAGCTAGACTAGTGGCCACCATGGGTTGTAGACTCCTG | |
| | TGCTGTGCAGTTTTGTGCCTTCTCGGGGCCGTGCCAATAGA | |
| | TACTGAAGTGACGCAAACCCCAAAGCACTTGGTAATGGGA | |
| | ATGACTAATAAAAAAAGCCTGAAGTGTGAGCAGCACATGG | |
| | GTCACCGAGCAATGTATTGGTACAAACAAAAAGCGAAGAA | |
| | GCCACCGGAGCTTATGTTTGTATATTCTTATGAGAAGTTGT | |
| | CAATCAATGAGAGCGTCCCCTCCCGGTTCTCACCGGAGTGT | |
| | CCTAACAGTAGCTTGCTCAATCTCCATCTCCATGCTCTCCA | |
| | GCCGGAAGACAGCGCTCTCTACCTTTGTGCAAGTTCTCCCA | |
| | CGAGCGGAATTTATGAACAGTACTTTGGGCCTGGTACGCG | |
| | CCTCACGGTCACTGAGGACTTGAAGAATGTCTTCCCTCCAG | |
| | AGGTTGCCGTGTTTGAGCCCTCAGAGGCTGAAATTTCTCAC | |
| | ACCCAAAAGGCCACACTCGTCTGTTTGGCTACGGGTTTCTA | |
| | TCCCGATCACGTGGAACTTTCTTGGTGGGTCAATGGAAAAG | |
| | AAGTTCACAGTGGTGTCAGTACAGACCCTCAGCCACTTAA | |
| | GGAGCAACCAGCTCTGAACGATTCTCGATACTGCTTGTCAT | |
| | CTCGCCTCAGAGTCAGCGCTACTTTCTGGCAAAACCCAAGA | |
| | AACCATTTCAGATGCCAAGTTCAGTTCTATGGCTTGTCCGA | |
| | GAACGATGAGTGGACCCAAGACCGCGCGAAGCCAGTTACA | |
| | CAGATAGTAAGCGCCGAAGCGTGGGGTCGCGCCGACTGTG | |
| | GCTTCACATCTGAGAGTTATCAGCAAGGAGTACTGAGTGC | |
| | AACTATTTTGTATGAGATATTGCTGGGAAAAGCGACGTTGT | |
| | ACGCCGTGCTGGTGTCCGCTCTCGTGCTCATGGCCATGGTA | |
| | AAGCGAAAGGATAGTAGGGGAGCTACGAACTTTTCACTCC | |
| | TGAAGCAAGCAGGTGACGTGGAGGAGAACCCAGGTCCGAT | |
| | GTTGTTGGAACTGATTCCTCTCCTTGGTATCCATTTCGTCCT | |
| | CCGAACCGCGCGGGCTCAGTCTGTCACTCAGCCAGATATA | |
| | CACATAACAGTTTCTGAGGGCGCTAGCTTGGAGCTTCGGTG | |
| | CAACTATAGTTACGGTGCCACACCGTACCTTTTCTGGTACG | |
| | TGCAGAGCCCCGGACAGGGGCTTCAACTCCTCCTGAAGTA | |
| | CTTTTCAGGGGACACTTTGGTTCAGGGAATTAAGGGTTTTG | |
| | AGGCTGAATTTAAGAGGTCACAGTCCAGCTTCAACTTGCG | |
| | AAAACCCTCAGTTCATTGGAGTGATGCCGCAGAGTATTTCT | |
| | GTGCAGTAGGAGCGCCATCTGGCGCTGGCTCATATCAATTG | |
| | ACCTTTGGGAAGGGAACCAAACTGAGCGTTATTCCCAATA | |
| | TCCAAAACCCAGACCCAGCGGTTTATCAATTGAGGGATTCC | |
| | AAATCTAGTGATAAATCAGTGTGCCTTTTTACTGACTTTGA | |
| | TAGTCAGACGAACGTATCCCAAAGCAAGGATAGTGACGTC | |
| | TACATAACAGATAAGACAGTTCTCGATATGCGCTCAATGG | |
| | ATTTTAAGTCTAACAGCGCGGTGGCATGGAGTAACAAGTC | |
| | CGACTTTGCCTGCGCGAATGCTTTCAATAACAGTATTATAC | |
| | CCGAAGACACGTTCTTTCCTTCACCCGAAAGTTCTTGTGAC | |
| | GTCAAGCTGGTCGAGAAAAGTTTCGAAACTGATACGAACC | |
| | TGAATTTCCAAAATTTGTCCGTTATTGGATTTCGAATACTTC | |
| | TTCTGAAAGTGGCGGGCTTCAATTTGTTGATGACATTGCGA | |
| | CTTTGGTCATCCTAA | |
| Carbonic anhydrase | MSHHWGYGKHNGPEHWHKDFPIAKGERQSPVDIDTHTAKYD | 30 |
| II (CA2) | PSLKPLSVSYDQATSLRILNNGHAFNVEFDDSQDKAVLKGGP | |
| | LDGTYRLIQFHFHWGSLDGQGSEHTVDKKKYAAELHLVHWN | |
| | TKYGDFGKAVQQPDGLAVLGIFLKVGSAKPGLQKVVDVLDSI | |
| | KTKGKSADFTNFDPRGLLPESLDYWTYPGSLTTPPLLECVTWI | |
| | VLKEPISVSSEQVLKFRKLNFNGEGEPEELMVDNWRPAQPLK | |
| | NRQIKASFK | |
| Human FKBP | GVQVETISPGDGRTFPKRGQTCVVHYTGMLEDGKKFDSSRDR | 31 |
| (FK506 binding | NKPFKFMLGKQEVIRGWEEGVAQMSVGQRAKLTISPDYAYG | |
| protein) | ATGHPGIIPPHATLVFDVELLKLE | |
| E. coli dihydrofolate | MISLIAALAVDRVIGMENAMPWNLPADLAWFKRNTLNKPVI | 32 |
| reductase (ecDHFR) | MGRHTWESIGRPLPGRKNIILSSQPGTDDRVTWVKSVDEAIAA | |
| | CGDVPEIMVIGGGRVYEQFLPKAQKLYLTHIDAEVEGDTHFP | |
| | DYEPDDWESVFSEFHDADAQNSHSYCFEILERR | |
| Human | MVGSLNCIVAVSQNMGIGKNGDLPWPPLRNEFRYFQRMTTTS | 33 |
| dihydrofolate | SVEGKQNLVIMGKKTWFSIPEKNRPLKGRINLVLSRELKEPPQ | |
| reductase (hDHFR) | GAHFLSRSLDDALKLTEQPELANKVDMVWIVGGSSVYKEAM | |
| | NHPGHLKLFVTRIMQDFESDTFFPEIDLEKYKLLPEYPGVLSD | |
| | VQEEKGIKYKFEVYEKND | |
| Human estrogen | MTMTLHTKASGMALLHQIQGNELEPLNRPQLKIPLERPLGEV | 34 |
| receptor (ER) | YLDSSKPAVYNYPEGAAYEFNAAAAANAQVYGQTGLPYGPG | |
| | SEAAAFGSNGLGGFPPLNSVSPSPLMLLHPPPQLSPFLQPHGQ | |
| | QVPYYLENEPSGYTVREAGPPAFYRPNSDNRRQGGRERLAST | |
| | NDKGSMAMESAKETRYCAVCNDYASGYHYGVWSCEGCKAF | |
| | FKRSIQGHNDYMCPATNQCTIDKNRRKSCQACRLRKCYEVG | |
| | MMKGGIRKDRRGGRMLKHKRQRDDGEGRGEVGSAGDMRA | |
| | ANLWPSPLMIKRSKKNSLALSLTADQMVSALLDAEPPILYSEY | |
| | DPTRPFSEASMMGLLTNLADRELVHMINWAKRVPGFVDLTL | |
| | HDQVHLLECAWLEILMIGLVWRSMEHPGKLLFAPNLLLDRN | |
| | QGKCVEGMVEIFDMLLATSSRFRMMNLQGEEFVCLKSIILLNS | |
| | GVYTFLSSTLKSLEEKDHIHRVLDKITDTLIHLMAKAGLTLQQ | |
| | QHQRLAQLLLILSHIRHMSNKGMEHLYSMKCKNVVPLYDLL | |
| | LEMLDAHRLHAPTSRGGASVEETDQSHLATAGSTSSHSLQKY | |
| | YITGEAEGFPATV | |
| Phosphodiesterase 5 | MERAGPSFGQQRQQQQPQQQKQQQRDQDSVEAWLDDHWD | 35 |
| (PDE5), full-length | FTFSYFVRKATREMVNAWFAERVHTIPVCKEGIRGHTESCSCP | |
| | LQQSPRADNSAPGTPTRKISASEFDRPLRPIVVKDSEGTVSFLS | |
| | DSEKKEQMPLTPPRFDHDEGDQCSRLLELVKDISSHLDVTALC | |
| | HKIFLHIHGLISADRYSLFLVCEDSSNDKFLISRLFDVAEGSTLE | |
| | EVSNNCIRLEWNKGIVGHVAALGEPLNIKDAYEDPRFNAEVD | |
| | QITGYKTQSILCMPIKNHREEVVGVAQAINKKSGNGGTFTEK | |
| | DEKDFAAYLAFCGIVLHNAQLYETSLLENKRNQVLLDLASLIF | |
| | EEQQSLEVILKKIAATIISFMQVQKCTIFIVDEDCSDSFSSVFHM | |
| | ECEELEKSSDTLTREHDANKINYMYAQYVKNTMEPLNIPDVS | |
| | KDKRFPWTTENTGNVNQQCIRSLLCTPIKNGKKNKVIGVCQL | |
| | VNKMEENTGKVKPFNRNDEQFLEAFVIFCGLGIQNTQMYEAV | |
| | ERAMAKQMVTLEVLSYHASAAEEETRELQSLAAAVVPSAQT | |
| | LKITDFSFSDFELSDLETALCTIRMFTDLNLVQNFQMKHEVLC | |
| | RWILSVKKNYRKNVAYHNWRHAFNTAQCMFAALKAGKIQN | |
| | KLTDLEILALLIAALSHDLDHRGVNNSYIQRSEHPLAQLYCHSI | |
| | MEHHHFDQCLMILNSPGNQILSGLSIEEYKTTLKIIKQAILATD | |
| | LALYIKRRGEFFELIRKNQFNLEDPHQKELFLAMLMTACDLSA | |
| | ITKPWPIQQRIAELVATEFFDQGDRERKELNIEPTDLMNREKK | |
| | NKIPSMQVGFIDAICLQLYEALTHVSEDCFPLLDGCRKNRQK | |
| | WQALAEQQEKMLINGESGQAKRN | |
| Phosphodiesterase 5 | MEETRELQSLAAAVVPSAQTLKITDFSFSDFELSDLETALCTIR | 36 |
| (PDE5), ligand | MFTDLNLVQNFQMKHEVLCRWILSVKKNYRKNVAYHNWRH | |
| binding domain | AFNTAQCMFAALKAGKIQNKLTDLEILALLIAALSHDLDHRG | |
| | VNNSYIQRSEHPLAQLYCHSIMEHHHFDQCLMILNSPGNQILS | |
| | GLSIEEYKTTLKIIKQAILATDLALYIKRRGEFFELIRKNQFNLE | |
| | DPHQKELFLAMLMTACDLSAITKPWPIQQRIAELVATEFFDQG | |
| | DRERKELNIEPTDLMNREKKNKIPSMQVGFIDAICLQLYEALT | |
| | HVSEDCFPLLDGCRKNRQKWQALAEQQ | |
| P65 transcription | CTGTCTGAGGCCCTGCTGCAGCTCCAGTTCGACGATGAAGA | 37 |
| factor activation | TCTGGGAGCCCTGCTGGGCAACAGCACAGATCCTGCCGTG | |
| domain | TTTACCGATCTGGCCAGCGTGGACAACAGCGAGTTTCAGC | |
| | AGCTCCTGAACCAGGGCATCCCTGTGGCTCCTCACACCACA | |
| | GAGCCCATGCTGATGGAATACCCCGAGGCCATCACCAGAC | |
| | TGGTCACCGGTGCTCAAAGACCTCCAGATCCAGCTCCTGCT | |
| | CCACTGGGAGCACCTGGACTGCCTAATGGACTGCTGTCTGG | |
| | CGACGAGGACTTCAGCTCTATCGCCGACATGGATTTCAGCG | |
| | CCCTGCTGAGCCAGATCAGCTCT | |
| ZFHD1 transcription | GATTATCCTGCCGCCAAGAGAGTGAAGCTGGACAGCAGAG | 38 |
| factor DNA binding | AGAGGCCCTACGCCTGTCCTGTGGAAAGCTGCGACAGACG | |
| domain | GTTCAGCAGAAGCGACGAGCTGACCCGGCACATCAGAATC | |
| | CACACCGGCCAGAAACCTTTCCAGTGCCGGATCTGCATGC | |
| | GGAACTTCAGCAGATCCGACCACCTGACCACACACATCAG | |
| | GACACACACAGGCGGAGGCAGACGGCGGAAGAAGAGAAC | |
| | CAGCATCGAGACAAACATCCGCGTGGCCCTGGAAAAGAGC | |
| | TTCCTGGAAAACCAGAAGCCTACCAGCGAAGAGATCACCA | |
| | TGATCGCCGACCAGCTGAACATGGAAAAAGAAGTGATCCG | |
| | CGTCTGGTTCTGCAACCGGCGGCAGAAAGAGAAGCGGATC | |
| | AACGGCAGCCGGAAGAGATGTGCTGCTGGTGTTGGCGGAG | |
| | GACCTGCTGGATGTCCTGCTCCTGGAAGCACCCCTCTGAAG | |
| | AAGCCTAGAAGA | |
| EGFP-038 | CGTGAGGCTCCGGTGCCCGTCAGTGGGCAGAGCGCACATC | 39 |
| (EF1a-EGFP-PEST) | GCCCACAGTCCCCGAGAAGTTGGGGGGAGGGGTCGGCAAT | |
| | TGAACCGGTGCCTAGAGAAGGTGGCGCGGGGTAAACTGGG | |
| | AAAGTGATGTCGTGTACTGGCTCCGCCTTTTTCCCGAGGGT | |
| | GGGGGAGAACCGTATATAAGTGCAGTAGTCGCCGTGAACG | |
| | TTCTTTTTCGCAACGGGTTTGCCGCCAGAACACAGGTAAGT | |
| | GCCGTGTGTGGTTCCCGCGGGCCTGGCCTCTTTACGGGTTA | |
| | TGGCCCTTGCGTGCCTTGAATTACTTCCACCTGGCTGCAGT | |
| | ACGTGATTCTTGATCCCGAGCTTCGGGTTGGAAGTGGGTGG | |
| | GAGAGTTCGAGGCCTTGCGCTTAAGGAGCCCCTTCGCCTCG | |
| | TGCTTGAGTTGAGGCCTGGCCTGGGCGCTGGGGCCGCCGC | |
| | GTGCGAATCTGGTGGCACCTTCGCGCCTGTCTCGCTGCTTT | |
| | CGATAAGTCTCTAGCCATTTAAAATTTTTGATGACCTGCTG | |
| | CGACGCTTTTTTTCTGGCAAGATAGTCTTGTAAATGCGGGC | |
| | CAAGATCTGCACACTGGTATTTCGGTTTTTGGGGCCGCGGG | |
| | CGGCGACGGGGCCCGTGCGTCCCAGCGCACATGTTCGGCG | |
| | AGGCGGGGCCTGCGAGCGCGGCCACCGAGAATCGGACGG | |
| | GGGTAGTCTCAAGCTGGCCGGCCTGCTCTGGTGCCTGGCCT | |
| | CGCGCCGCCGTGTATCGCCCCGCCCTGGGCGGCAAGGCTG | |
| | GCCCGGTCGGCACCAGTTGCGTGAGCGGAAAGATGGCCGC | |
| | TTCCCGGCCCTGCTGCAGGGAGCTCAAAATGGAGGACGCG | |
| | GCGCTCGGGAGAGCGGGCGGGTGAGTCACCCACACAAAGG | |
| | AAAAGGGCCTTTCCGTCCTCAGCCGTCGCTTCATGTGACTC | |
| | CACTGAGTACCGGGCGCCGTCCAGGCACCTCGATTAGTTCT | |
| | CGAGCTTTTGGAGTACGTCGTCTTTAGGTTGGGGGGAGGG | |
| | GTTTTATGCGATGGAGTTTCCCCACACTGAGTGGGTGGAGA | |
| | CTGAAGTTAGGCCAGCTTGGCACTTGATGTAATTCTCCTTG | |
| | GAATTTGCCCTTTTTGAGTTTGGATCTTGGTTCATTCTCAAG | |
| | CCTCAGACAGTGGTTCAAAGTTTTTTTCTTCCATTTCAGGTG | |
| | TCGTGATCTAGAGGATCACTAGTGCCACCATGGTGAGCAA | |
| | GGGCGAGGAGCTGTTCACCGGGGTGGTGCCCATCCTGGTC | |
| | GAGCTGGACGGCGACGTAAACGGCCACAAGTTCAGCGTGT | |
| | CCGGCGAGGGCGAGGGCGATGCCACCTACGGCAAGCTGAC | |
| | CCTGAAGTTCATCTGCACCACCGGCAAGCTGCCCGTGCCCT | |
| | GGCCCACCCTCGTGACCACCCTGACCTACGGCGTGCAGTGC | |
| | TTCAGCCGCTACCCCGACCACATGAAGCAGCACGACTTCTT | |
| | CAAGTCCGCCATGCCCGAAGGCTACGTCCAGGAGCGCACC | |
| | ATCTTCTTCAAGGACGACGGCAACTACAAGACCCGCGCCG | |
| | AGGTGAAGTTCGAGGGCGACACCCTGGTGAACCGCATCGA | |
| | GCTGAAGGGCATCGACTTCAAGGAGGACGGCAACATCCTG | |
| | GGGCACAAGCTGGAGTACAACTACAACAGCCACAACGTCT | |
| | ATATCATGGCCGACAAGCAGAAGAACGGCATCAAGGTGAA | |
| | CTTCAAGATCCGCCACAACATCGAGGACGGCAGCGTGCAG | |
| | CTCGCCGACCACTACCAGCAGAACACCCCCATCGGCGACG | |
| | GCCCCGTGCTGCTGCCCGACAACCACTACCTGAGCACCCA | |
| | GTCCAAGCTGAGCAAAGACCCCAACGAGAAGCGCGATCAC | |
| | ATGGTCCTGCTGGAGTTCGTGACCGCCGCCGGGATCACTCT | |
| | CGGCATGGACGAGCTGTACAAGAAGCTTAGCCATGGCTTC | |
| | CCGCCGGAGGTGGAGGAGCAGGATGATGGCACGCTGCCCA | |
| | TGTCTTGTGCCCAGGAGAGCGGGATGGACCGTCACCCTGC | |
| | AGCCTGTGCTTCTGCTAGGATCAATGTG | |
| ZFHD1 binding | TAATGATGGGCG | 40 |
| sequence | | |
| LCK shRNA | GCATGAACTGGTCCGCCATTA | 41 |
| (TXN-123) | | |
| LCK shRNA | GCCATTAACTACGGGACATTC | 42 |
| (TXN-124) | | |
| LCK shRNA | TTCATTGAAGAGCGGAATTAT | 43 |
| (TXN-125) | | |
| TXN-076 | TAATGATGGGCGCTCGAGTAATGATGGGCGGTCGACTAAT | 44 |
| (12xZHFD1-YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| EGFP-EFS-Nls- | AGTAATGATGGGCGGTCGACTAATGATGGGCGCTCGAGTA | |
| Zfhd1-ierNLS-p65- | ATGATGGGCGTCTAGAATCTAGAGGGTATATAATGGGGGC | |
| GGSGGGSG- | CAACCGGTCCTGCAGCGGAGGATCCGCCACCATGTTGAGC | |
| hDHFR(134)-MND- | AAGGGCGAGGAGGACAACATGGCCATCATCAAGGAGTTCA | |
| CD19CAR) | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGTCAAGCTGACCCTGAAGTTCA | |
| | TTAGTGAAGCCACAGATGTAATGAACTTCAGGGTCAGCTT | |
| | GCTGCCTACTGCCTCGGACTTCAAGGGACGCGTTGGCTTAC | |
| | CTCCCATTGACCTTATGTACTGGGCAAAACCCATTGGAAAG | |
| | TCCCTATTGACTCAATGTACTTGGCTCCAATGGGACTTTCC | |
| | TGTTGACTCACCCCCTATTGACCTTATGTACTGGGCAAAAC | |
| | CCAATGGAAAGTCCCTATTGAGTCAGTGTACTTGGCTCCAA | |
| | AGGGTTTTTCCCATTGACTAGTCGAGGAAGAGCATGCGTG | |
| | AGGCTCCGGTGCCCGTCAGTGGGCAGAGCGCACATCGCCC | |
| | ACAGTCCCCGAGAAGTTGGGGGGAGGGGTCGGCAATTGAA | |
| | CCGGTGCCTAGAGAAGGTGGCGCGGGGTAAACTGGGAAAG | |
| | TGATGTCGTGTACTGGCTCCGCCTTTTTCCCGAGGGTGGGG | |
| | GAGAACCGTATATAAGTGCAGTAGTCGCCGTGAACGTTCTT | |
| | TTTCGCAACGGGTTTGCCGCCAGAACACAGGCTAGACTAG | |
| | TGCCACCATGGATTATCCTGCCGCCAAGAGAGTGAAGCTG | |
| | GACAGCAGAGAGAGGCCCTACGCCTGTCCTGTGGAAAGCT | |
| | GCGACAGACGGTTCAGCAGAAGCGACGAGCTGACCCGGCA | |
| | CATCAGAATCCACACCGGCCAGAAACCTTTCCAGTGCCGG | |
| | ATCTGCATGCGGAACTTCAGCAGATCCGACCACCTGACCA | |
| | CACACATCAGGACACACACAGGCGGAGGCAGACGGCGGA | |
| | AGAAGAGAACCAGCATCGAGACAAACATCCGCGTGGCCCT | |
| | GGAAAAGAGCTTCCTGGAAAACCAGAAGCCTACCAGCGAA | |
| | GAGATCACCATGATCGCCGACCAGCTGAACATGGAAAAAG | |
| | AAGTGATCCGCGTCTGGTTCTGCAACCGGCGGCAGAAAGA | |
| | GAAGCGGATCAACGGCAGCCGGAAGAGATGTGCTGCTGGT | |
| | GTTGGCGGAGGACCTGCTGGATGTCCTGCTCCTGGAAGCA | |
| | CCCCTCTGAAGAAGCCTAGAAGAGGCAGCCTGTCTGAGGC | |
| | CCTGCTGCAGCTCCAGTTCGACGATGAAGATCTGGGAGCC | |
| | CTGCTGGGCAACAGCACAGATCCTGCCGTGTTTACCGATCT | |
| | GGCCAGCGTGGACAACAGCGAGTTTCAGCAGCTCCTGAAC | |
| | CAGGGCATCCCTGTGGCTCCTCACACCACAGAGCCCATGCT | |
| | GATGGAATACCCCGAGGCCATCACCAGACTGGTCACCGGT | |
| | GCTCAAAGACCTCCAGATCCAGCTCCTGCTCCACTGGGAGC | |
| | ACCTGGACTGCCTAATGGACTGCTGTCTGGCGACGAGGAC | |
| | TTCAGCTCTATCGCCGACATGGATTTCAGCGCCCTGCTGAG | |
| | CCAGATCAGCTCTGGAGGTAGTGGTGGCGGATCCGGAATG | |
| | GTGGGCTCTCTGAATTGCATTGTGGCCGTGTCTCAGAACAT | |
| | GGGCATCGGCAAGAACGGCGACCTTCCTTGGCCTCCTCTGC | |
| | GGAACGAGCTGCGGTACTTCCACAGAATGACCACCACCAG | |
| | CAGCGTGGAAGGCAAGCAGAACCTGGTCATCATGGGCAAG | |
| | AAAACCTGGTTCAGCATCGGCAGACCCCTGAAGGGCAGAA | |
| | TCAACCTGGTGCTGAGCAGAGAGCTGAAAGAGCCTCCTCA | |
| | GGGCGCCCACTTTCTGAGCAGATCTCTGGACGATGCCCTGA | |
| | AGCTGACCGAGCAGCCTGAACTGGCTAACAAGGTGGACAT | |
| | GGTCTGGATCGTCGGCGGCAGCTCCGTGATCAAAGAAGCC | |
| | ATGAATCACCCCGGCCACCTGAAACTGTTCGTGACCAGAA | |
| | TCATGCAGGACTTCGAGTCCGACACATTCTTCCCAGAGATC | |
| | GACCTGGAAAAGTACAAGCTGCTGCCAGAGTACCCCGGCG | |
| | TGCTGTCCGATGTGCAAGAGGAAAAGGGCATCAAGTACAA | |
| | GTTCGAGGTGTACGAGAAGAACGATTAAGCTAGCGAACAG | |
| | AGAAACAGGAGAATATGGGCCAAACAGGATATCTGTGGTA | |
| | AGCAGTTCCTGCCCCGGCTCAGGGCCAAGAACAGTTGGAA | |
| | CAGCAGAATATGGGCCAAACAGGATATCTGTGGTAAGCAG | |
| | TTCCTGCCCCGGCTCAGGGCCAAGAACAGATGGTCCCCAG | |
| | ATGCGGTCCCGCCCTCAGCAGTTTCTAGAGAACCATCAGAT | |
| | GTTTCCAGGGTGCCCCAAGGACCTGAAATGACCCTGTGCCT | |
| | TATTTGAACTAACCAATCAGTTCGCTTCTCGCTTCTGTTCGC | |
| | GCGCTTCTGCTCCCCGAGCTCTATATAAGCAGAGCTCGTTT | |
| | AGTGAACCGTCAGATCGCCACCACTAGTGCCACCATGGCT | |
| | CTGCCTGTGACAGCTCTGCTGCTGCCTCTGGCTCTGCTTCTG | |
| | CATGCCGCCAGACCTGACATCCAGATGACCCAGACAACCA | |
| | GCAGCCTGTCTGCCAGCCTGGGCGATAGAGTGACCATCAG | |
| | CTGTAGAGCCAGCCAGGACATCAGCAAGTACCTGAACTGG | |
| | TATCAGCAGAAACCCGACGGCACCGTGAAGCTGCTGATCT | |
| | ACCACACCAGCAGACTGCACAGCGGCGTGCCAAGCAGATT | |
| | TTCTGGCAGCGGCTCTGGCACCGACTACAGCCTGACAATCA | |
| | GCAACCTGGAACAAGAGGATATCGCTACCTACTTCTGCCA | |
| | GCAAGGCAACACCCTGCCTTACACCTTTGGCGGAGGCACC | |
| | AAGCTGGAAATCACAGGCGGCGGAGGAAGCGGAGGCGGA | |
| | GGATCTGGTGGTGGTGGATCTGAAGTGAAACTGCAAGAGT | |
| | CTGGCCCTGGCCTGGTGGCCCCATCTCAATCTCTGAGCGTG | |
| | ACCTGTACCGTCAGCGGAGTGTCCCTGCCTGATTATGGCGT | |
| | GTCCTGGATTCGGCAGCCTCCTAGAAAAGGCCTGGAATGG | |
| | CTGGGCGTGATCTGGGGCAGCGAGACAACCTACTACAACA | |
| | GCGCCCTGAAGTCCCGGCTGACCATCATCAAGGACAACTC | |
| | CAAGAGCCAGGTGTTCCTGAAGATGAACAGCCTGCAGACC | |
| | GACGACACCGCCATCTACTATTGCGCCAAGCACTACTACTA | |
| | CGGCGGCAGCTACGCCATGGATTATTGGGGCCAGGGCACC | |
| | AGCGTGACCGTGTCTAGTACAACAACCCCTGCTCCTCGGCC | |
| | TCCTACACCAGCTCCTACAATTGCCAGCCAGCCACTGTCTC | |
| | TGAGGCCCGAAGCTTGTAGACCTGCTGCTGGCGGAGCCGT | |
| | GCATACAAGAGGACTGGATTTCGCCTGCGACATCTACATCT | |
| | GGGCCCCTCTGGCTGGAACATGTGGCGTGCTGCTGCTGAGC | |
| | CTGGTCATCACCCTGTATTGCAAGCGGGGCAGAAAGAAAC | |
| | TGCTCTACATCTTCAAGCAGCCCTTCATGCGGCCCGTGCAG | |
| | ACCACACAAGAGGAAGATGGCTGCTCCTGCAGATTCCCCG | |
| | AGGAAGAAGAAGGCGGCTGCGAGCTGAGAGTGAAGTTCA | |
| | GCAGATCCGCCGACGCTCCCGCCTATAAGCAGGGACAGAA | |
| | CCAGCTGTACAACGAGCTGAACCTGGGGAGAAGAGAAGA | |
| | GTACGACGTGCTGGACAAGCGGAGAGGCAGAGATCCTGAA | |
| | ATGGGCGGCAAGCCCAGACGGAAGAATCCTCAAGAGGGCC | |
| | TGTATAATGAGCTGCAGAAAGACAAGATGGCCGAGGCCTA | |
| | CAGCGAGATCGGAATGAAGGGCGAGCGCAGAAGAGGCAA | |
| | GGGACACGATGGACTGTACCAGGGCCTGAGCACCGCCACC | |
| | AAGGATACCTATGATGCCCTGCACATGCAGGCCCTGCCTCC | |
| | AAGATAA | |
| TXN-080 | TAATGATGGGCGCTCGAGTAATGATGGGCGGTCGACTAAT | 45 |
| (12xZHFD1-YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| LAT-EFS-Nls- | AGTAATGATGGGCGGTCGACTAATGATGGGCGCTCGAGTA | |
| Zfhd1-ierNLS-p65- | ATGATGGGCGTCTAGAATCTAGAGGGTATATAATGGGGGC | |
| GGSGGGSG- | CAACCGGTCCTGCAGCGGAGGATCCGCCACCATGTTGAGC | |
| hDHFR(134)-MND- | AAGGGCGAGGAGGACAACATGGCCATCATCAAGGAGTTCA | |
| CD19CAR) | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGCCTACTCTGTGTAATAGAATA | |
| | TAGTGAAGCCACAGATGTATATTCTATTACACAGAGTAGGT | |
| | GCCTACTGCCTCGGACTTCAAGGGACGCGTTGGCTTACCTC | |
| | CCATTGACCTTATGTACTGGGCAAAACCCATTGGAAAGTCC | |
| | CTATTGACTCAATGTACTTGGCTCCAATGGGACTTTCCTGT | |
| | TGACTCACCCCCTATTGACCTTATGTACTGGGCAAAACCCA | |
| | ATGGAAAGTCCCTATTGAGTCAGTGTACTTGGCTCCAAAGG | |
| | GTTTTTCCCATTGACTAGTCGAGGAAGAGCATGCGTGAGGC | |
| | TCCGGTGCCCGTCAGTGGGCAGAGCGCACATCGCCCACAG | |
| | TCCCCGAGAAGTTGGGGGGAGGGGTCGGCAATTGAACCGG | |
| | TGCCTAGAGAAGGTGGCGCGGGGTAAACTGGGAAAGTGAT | |
| | GTCGTGTACTGGCTCCGCCTTTTTCCCGAGGGTGGGGGAGA | |
| | ACCGTATATAAGTGCAGTAGTCGCCGTGAACGTTCTTTTTC | |
| | GCAACGGGTTTGCCGCCAGAACACAGGCTAGACTAGTGCC | |
| | ACCATGGATTATCCTGCCGCCAAGAGAGTGAAGCTGGACA | |
| | GCAGAGAGAGGCCCTACGCCTGTCCTGTGGAAAGCTGCGA | |
| | CAGACGGTTCAGCAGAAGCGACGAGCTGACCCGGCACATC | |
| | AGAATCCACACCGGCCAGAAACCTTTCCAGTGCCGGATCT | |
| | GCATGCGGAACTTCAGCAGATCCGACCACCTGACCACACA | |
| | CATCAGGACACACACAGGCGGAGGCAGACGGCGGAAGAA | |
| | GAGAACCAGCATCGAGACAAACATCCGCGTGGCCCTGGAA | |
| | AAGAGCTTCCTGGAAAACCAGAAGCCTACCAGCGAAGAGA | |
| | TCACCATGATCGCCGACCAGCTGAACATGGAAAAAGAAGT | |
| | GATCCGCGTCTGGTTCTGCAACCGGCGGCAGAAAGAGAAG | |
| | CGGATCAACGGCAGCCGGAAGAGATGTGCTGCTGGTGTTG | |
| | GCGGAGGACCTGCTGGATGTCCTGCTCCTGGAAGCACCCCT | |
| | CTGAAGAAGCCTAGAAGAGGCAGCCTGTCTGAGGCCCTGC | |
| | TGCAGCTCCAGTTCGACGATGAAGATCTGGGAGCCCTGCT | |
| | GGGCAACAGCACAGATCCTGCCGTGTTTACCGATCTGGCC | |
| | AGCGTGGACAACAGCGAGTTTCAGCAGCTCCTGAACCAGG | |
| | GCATCCCTGTGGCTCCTCACACCACAGAGCCCATGCTGATG | |
| | GAATACCCCGAGGCCATCACCAGACTGGTCACCGGTGCTC | |
| | AAAGACCTCCAGATCCAGCTCCTGCTCCACTGGGAGCACCT | |
| | GGACTGCCTAATGGACTGCTGTCTGGCGACGAGGACTTCA | |
| | GCTCTATCGCCGACATGGATTTCAGCGCCCTGCTGAGCCAG | |
| | ATCAGCTCTGGAGGTAGTGGTGGCGGATCCGGAATGGTGG | |
| | GCTCTCTGAATTGCATTGTGGCCGTGTCTCAGAACATGGGC | |
| | ATCGGCAAGAACGGCGACCTTCCTTGGCCTCCTCTGCGGAA | |
| | CGAGCTGCGGTACTTCCACAGAATGACCACCACCAGCAGC | |
| | GTGGAAGGCAAGCAGAACCTGGTCATCATGGGCAAGAAAA | |
| | CCTGGTTCAGCATCGGCAGACCCCTGAAGGGCAGAATCAA | |
| | CCTGGTGCTGAGCAGAGAGCTGAAAGAGCCTCCTCAGGGC | |
| | GCCCACTTTCTGAGCAGATCTCTGGACGATGCCCTGAAGCT | |
| | GACCGAGCAGCCTGAACTGGCTAACAAGGTGGACATGGTC | |
| | TGGATCGTCGGCGGCAGCTCCGTGATCAAAGAAGCCATGA | |
| | ATCACCCCGGCCACCTGAAACTGTTCGTGACCAGAATCATG | |
| | CAGGACTTCGAGTCCGACACATTCTTCCCAGAGATCGACCT | |
| | GGAAAAGTACAAGCTGCTGCCAGAGTACCCCGGCGTGCTG | |
| | TCCGATGTGCAAGAGGAAAAGGGCATCAAGTACAAGTTCG | |
| | AGGTGTACGAGAAGAACGATTAAGCTAGCGAACAGAGAA | |
| | ACAGGAGAATATGGGCCAAACAGGATATCTGTGGTAAGCA | |
| | GTTCCTGCCCCGGCTCAGGGCCAAGAACAGTTGGAACAGC | |
| | AGAATATGGGCCAAACAGGATATCTGTGGTAAGCAGTTCC | |
| | TGCCCCGGCTCAGGGCCAAGAACAGATGGTCCCCAGATGC | |
| | GGTCCCGCCCTCAGCAGTTTCTAGAGAACCATCAGATGTTT | |
| | CCAGGGTGCCCCAAGGACCTGAAATGACCCTGTGCCTTATT | |
| | TGAACTAACCAATCAGTTCGCTTCTCGCTTCTGTTCGCGCG | |
| | CTTCTGCTCCCCGAGCTCTATATAAGCAGAGCTCGTTTAGT | |
| | GAACCGTCAGATCGCCACCACTAGTGCCACCATGGCTCTGC | |
| | CTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |
| TXN-081 | TAATGATGGGCGCTCGAGTAATGATGGGCGGTCGACTAAT | 46 |
| (12xZHFD1-YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| LAT-EFS-Nls- | AGTAATGATGGGCGGTCGACTAATGATGGGCGCTCGAGTA | |
| Zfhd1-ierNLS-p65- | ATGATGGGCGTCTAGAATCTAGAGGGTATATAATGGGGGC | |
| GGSGGGSG- | CAACCGGTCCTGCAGCGGAGGATCCGCCACCATGTTGAGC | |
| hDHFR(134)-MND- | AAGGGCGAGGAGGACAACATGGCCATCATCAAGGAGTTCA | |
| CD19CAR) | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGGGGCTCCAGATTACGAGAAT | |
| | CTAGTGAAGCCACAGATGTAGATTCTCGTAATCTGGAGCCC | |
| | TGCCTACTGCCTCGGACTTCAAGGGACGCGTTGGCTTACCT | |
| | CCCATTGACCTTATGTACTGGGCAAAACCCATTGGAAAGTC | |
| | CCTATTGACTCAATGTACTTGGCTCCAATGGGACTTTCCTG | |
| | TTGACTCACCCCCTATTGACCTTATGTACTGGGCAAAACCC | |
| | AATGGAAAGTCCCTATTGAGTCAGTGTACTTGGCTCCAAAG | |
| | GGTTTTTCCCATTGACTAGTCGAGGAAGAGCATGCGTGAG | |
| | GCTCCGGTGCCCGTCAGTGGGCAGAGCGCACATCGCCCAC | |
| | AGTCCCCGAGAAGTTGGGGGGAGGGGTCGGCAATTGAACC | |
| | GGTGCCTAGAGAAGGTGGCGCGGGGTAAACTGGGAAAGTG | |
| | ATGTCGTGTACTGGCTCCGCCTTTTTCCCGAGGGTGGGGGA | |
| | GAACCGTATATAAGTGCAGTAGTCGCCGTGAACGTTCTTTT | |
| | TCGCAACGGGTTTGCCGCCAGAACACAGGCTAGACTAGTG | |
| | CCACCATGGATTATCCTGCCGCCAAGAGAGTGAAGCTGGA | |
| | CAGCAGAGAGAGGCCCTACGCCTGTCCTGTGGAAAGCTGC | |
| | GACAGACGGTTCAGCAGAAGCGACGAGCTGACCCGGCACA | |
| | TCAGAATCCACACCGGCCAGAAACCTTTCCAGTGCCGGAT | |
| | CTGCATGCGGAACTTCAGCAGATCCGACCACCTGACCACA | |
| | CACATCAGGACACACACAGGCGGAGGCAGACGGCGGAAG | |
| | AAGAGAACCAGCATCGAGACAAACATCCGCGTGGCCCTGG | |
| | AAAAGAGCTTCCTGGAAAACCAGAAGCCTACCAGCGAAGA | |
| | GATCACCATGATCGCCGACCAGCTGAACATGGAAAAAGAA | |
| | GTGATCCGCGTCTGGTTCTGCAACCGGCGGCAGAAAGAGA | |
| | AGCGGATCAACGGCAGCCGGAAGAGATGTGCTGCTGGTGT | |
| | TGGCGGAGGACCTGCTGGATGTCCTGCTCCTGGAAGCACC | |
| | CCTCTGAAGAAGCCTAGAAGAGGCAGCCTGTCTGAGGCCC | |
| | TGCTGCAGCTCCAGTTCGACGATGAAGATCTGGGAGCCCT | |
| | GCTGGGCAACAGCACAGATCCTGCCGTGTTTACCGATCTGG | |
| | CCAGCGTGGACAACAGCGAGTTTCAGCAGCTCCTGAACCA | |
| | GGGCATCCCTGTGGCTCCTCACACCACAGAGCCCATGCTGA | |
| | TGGAATACCCCGAGGCCATCACCAGACTGGTCACCGGTGC | |
| | TCAAAGACCTCCAGATCCAGCTCCTGCTCCACTGGGAGCAC | |
| | CTGGACTGCCTAATGGACTGCTGTCTGGCGACGAGGACTTC | |
| | AGCTCTATCGCCGACATGGATTTCAGCGCCCTGCTGAGCCA | |
| | GATCAGCTCTGGAGGTAGTGGTGGCGGATCCGGAATGGTG | |
| | GGCTCTCTGAATTGCATTGTGGCCGTGTCTCAGAACATGGG | |
| | CATCGGCAAGAACGGCGACCTTCCTTGGCCTCCTCTGCGGA | |
| | ACGAGCTGCGGTACTTCCACAGAATGACCACCACCAGCAG | |
| | CGTGGAAGGCAAGCAGAACCTGGTCATCATGGGCAAGAAA | |
| | ACCTGGTTCAGCATCGGCAGACCCCTGAAGGGCAGAATCA | |
| | ACCTGGTGCTGAGCAGAGAGCTGAAAGAGCCTCCTCAGGG | |
| | CGCCCACTTTCTGAGCAGATCTCTGGACGATGCCCTGAAGC | |
| | TGACCGAGCAGCCTGAACTGGCTAACAAGGTGGACATGGT | |
| | CTGGATCGTCGGCGGCAGCTCCGTGATCAAAGAAGCCATG | |
| | AATCACCCCGGCCACCTGAAACTGTTCGTGACCAGAATCAT | |
| | GCAGGACTTCGAGTCCGACACATTCTTCCCAGAGATCGACC | |
| | TGGAAAAGTACAAGCTGCTGCCAGAGTACCCCGGCGTGCT | |
| | GTCCGATGTGCAAGAGGAAAAGGGCATCAAGTACAAGTTC | |
| | GAGGTGTACGAGAAGAACGATTAAGCTAGCGAACAGAGA | |
| | AACAGGAGAATATGGGCCAAACAGGATATCTGTGGTAAGC | |
| | AGTTCCTGCCCCGGCTCAGGGCCAAGAACAGTTGGAACAG | |
| | CAGAATATGGGCCAAACAGGATATCTGTGGTAAGCAGTTC | |
| | CTGCCCCGGCTCAGGGCCAAGAACAGATGGTCCCCAGATG | |
| | CGGTCCCGCCCTCAGCAGTTTCTAGAGAACCATCAGATGTT | |
| | TCCAGGGTGCCCCAAGGACCTGAAATGACCCTGTGCCTTAT | |
| | TTGAACTAACCAATCAGTTCGCTTCTCGCTTCTGTTCGCGC | |
| | GCTTCTGCTCCCCGAGCTCTATATAAGCAGAGCTCGTTTAG | |
| | TGAACCGTCAGATCGCCACCACTAGTGCCACCATGGCTCTG | |
| | CCTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |
| TXN-082 | TAATGATGGGCGCTCGAGTAATGATGGGCGGTCGACTAAT | 47 |
| (12xZHFD1-YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| LAT-EFS-Nls- | AGTAATGATGGGCGGTCGACTAATGATGGGCGCTCGAGTA | |
| Zfhd1-ierNLS-p65- | ATGATGGGCGTCTAGAATCTAGAGGGTATATAATGGGGGC | |
| GGSGGGSG- | CAACCGGTCCTGCAGCGGAGGATCCGCCACCATGTTGAGC | |
| hDHFR(134)-MND- | AAGGGCGAGGAGGACAACATGGCCATCATCAAGGAGTTCA | |
| CD19CAR) | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGGTCCATTGATGATTACGTGAA | |
| | TAGTGAAGCCACAGATGTATTCACGTAATCATCAATGGACT | |
| | GCCTACTGCCTCGGACTTCAAGGGACGCGTTGGCTTACCTC | |
| | CCATTGACCTTATGTACTGGGCAAAACCCATTGGAAAGTCC | |
| | CTATTGACTCAATGTACTTGGCTCCAATGGGACTTTCCTGT | |
| | TGACTCACCCCCTATTGACCTTATGTACTGGGCAAAACCCA | |
| | ATGGAAAGTCCCTATTGAGTCAGTGTACTTGGCTCCAAAGG | |
| | GTTTTTCCCATTGACTAGTCGAGGAAGAGCATGCGTGAGGC | |
| | TCCGGTGCCCGTCAGTGGGCAGAGCGCACATCGCCCACAG | |
| | TCCCCGAGAAGTTGGGGGGAGGGGTCGGCAATTGAACCGG | |
| | TGCCTAGAGAAGGTGGCGCGGGGTAAACTGGGAAAGTGAT | |
| | GTCGTGTACTGGCTCCGCCTTTTTCCCGAGGGTGGGGGAGA | |
| | ACCGTATATAAGTGCAGTAGTCGCCGTGAACGTTCTTTTTC | |
| | GCAACGGGTTTGCCGCCAGAACACAGGCTAGACTAGTGCC | |
| | ACCATGGATTATCCTGCCGCCAAGAGAGTGAAGCTGGACA | |
| | GCAGAGAGAGGCCCTACGCCTGTCCTGTGGAAAGCTGCGA | |
| | CAGACGGTTCAGCAGAAGCGACGAGCTGACCCGGCACATC | |
| | AGAATCCACACCGGCCAGAAACCTTTCCAGTGCCGGATCT | |
| | GCATGCGGAACTTCAGCAGATCCGACCACCTGACCACACA | |
| | CATCAGGACACACACAGGCGGAGGCAGACGGCGGAAGAA | |
| | GAGAACCAGCATCGAGACAAACATCCGCGTGGCCCTGGAA | |
| | AAGAGCTTCCTGGAAAACCAGAAGCCTACCAGCGAAGAGA | |
| | TCACCATGATCGCCGACCAGCTGAACATGGAAAAAGAAGT | |
| | GATCCGCGTCTGGTTCTGCAACCGGCGGCAGAAAGAGAAG | |
| | CGGATCAACGGCAGCCGGAAGAGATGTGCTGCTGGTGTTG | |
| | GCGGAGGACCTGCTGGATGTCCTGCTCCTGGAAGCACCCCT | |
| | CTGAAGAAGCCTAGAAGAGGCAGCCTGTCTGAGGCCCTGC | |
| | TGCAGCTCCAGTTCGACGATGAAGATCTGGGAGCCCTGCT | |
| | GGGCAACAGCACAGATCCTGCCGTGTTTACCGATCTGGCC | |
| | AGCGTGGACAACAGCGAGTTTCAGCAGCTCCTGAACCAGG | |
| | GCATCCCTGTGGCTCCTCACACCACAGAGCCCATGCTGATG | |
| | GAATACCCCGAGGCCATCACCAGACTGGTCACCGGTGCTC | |
| | AAAGACCTCCAGATCCAGCTCCTGCTCCACTGGGAGCACCT | |
| | GGACTGCCTAATGGACTGCTGTCTGGCGACGAGGACTTCA | |
| | GCTCTATCGCCGACATGGATTTCAGCGCCCTGCTGAGCCAG | |
| | ATCAGCTCTGGAGGTAGTGGTGGCGGATCCGGAATGGTGG | |
| | GCTCTCTGAATTGCATTGTGGCCGTGTCTCAGAACATGGGC | |
| | ATCGGCAAGAACGGCGACCTTCCTTGGCCTCCTCTGCGGAA | |
| | CGAGCTGCGGTACTTCCACAGAATGACCACCACCAGCAGC | |
| | GTGGAAGGCAAGCAGAACCTGGTCATCATGGGCAAGAAAA | |
| | CCTGGTTCAGCATCGGCAGACCCCTGAAGGGCAGAATCAA | |
| | CCTGGTGCTGAGCAGAGAGCTGAAAGAGCCTCCTCAGGGC | |
| | GCCCACTTTCTGAGCAGATCTCTGGACGATGCCCTGAAGCT | |
| | GACCGAGCAGCCTGAACTGGCTAACAAGGTGGACATGGTC | |
| | TGGATCGTCGGCGGCAGCTCCGTGATCAAAGAAGCCATGA | |
| | ATCACCCCGGCCACCTGAAACTGTTCGTGACCAGAATCATG | |
| | CAGGACTTCGAGTCCGACACATTCTTCCCAGAGATCGACCT | |
| | GGAAAAGTACAAGCTGCTGCCAGAGTACCCCGGCGTGCTG | |
| | TCCGATGTGCAAGAGGAAAAGGGCATCAAGTACAAGTTCG | |
| | AGGTGTACGAGAAGAACGATTAAGCTAGCGAACAGAGAA | |
| | ACAGGAGAATATGGGCCAAACAGGATATCTGTGGTAAGCA | |
| | GTTCCTGCCCCGGCTCAGGGCCAAGAACAGTTGGAACAGC | |
| | AGAATATGGGCCAAACAGGATATCTGTGGTAAGCAGTTCC | |
| | TGCCCCGGCTCAGGGCCAAGAACAGATGGTCCCCAGATGC | |
| | GGTCCCGCCCTCAGCAGTTTCTAGAGAACCATCAGATGTTT | |
| | CCAGGGTGCCCCAAGGACCTGAAATGACCCTGTGCCTTATT | |
| | TGAACTAACCAATCAGTTCGCTTCTCGCTTCTGTTCGCGCG | |
| | CTTCTGCTCCCCGAGCTCTATATAAGCAGAGCTCGTTTAGT | |
| | GAACCGTCAGATCGCCACCACTAGTGCCACCATGGCTCTGC | |
| | CTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |
| TXN-123 | TAATGATGGGCGCTCGAGTAATGATGGGCGGTCGACTAAT | 48 |
| (12xZHFD1-YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| Lck-EFS-Nls-Zfhd1- | AGTAATGATGGGCGGTCGACTAATGATGGGCGCTCGAGTA | |
| ierNLS-p65- | ATGATGGGCGTCTAGAATCTAGAGGGTATATAATGGGGGC | |
| GGSGGGSG- | CAACCGGTCCTGCAGCGGAGGATCCGCCACCATGTTGAGC | |
| hDHFR(134)-MND- | AAGGGCGAGGAGGACAACATGGCCATCATCAAGGAGTTCA | |
| CD19CAR) | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGGCATGAACTGGTCCGCCATTA | |
| | TAGTGAAGCCACAGATGTATAATGGCGGACCAGTTCATGC | |
| | TGCCTACTGCCTCGGACTTCAAGGGACGCGTTGGCTTACCT | |
| | CCCATTGACCTTATGTACTGGGCAAAACCCATTGGAAAGTC | |
| | CCTATTGACTCAATGTACTTGGCTCCAATGGGACTTTCCTG | |
| | TTGACTCACCCCCTATTGACCTTATGTACTGGGCAAAACCC | |
| | AATGGAAAGTCCCTATTGAGTCAGTGTACTTGGCTCCAAAG | |
| | GGTTTTTCCCATTGACTAGTCGAGGAAGAGCATGCGTGAG | |
| | GCTCCGGTGCCCGTCAGTGGGCAGAGCGCACATCGCCCAC | |
| | AGTCCCCGAGAAGTTGGGGGGAGGGGTCGGCAATTGAACC | |
| | GGTGCCTAGAGAAGGTGGCGCGGGGTAAACTGGGAAAGTG | |
| | ATGTCGTGTACTGGCTCCGCCTTTTTCCCGAGGGTGGGGGA | |
| | GAACCGTATATAAGTGCAGTAGTCGCCGTGAACGTTCTTTT | |
| | TCGCAACGGGTTTGCCGCCAGAACACAGGCTAGACTAGTG | |
| | CCACCATGGATTATCCTGCCGCCAAGAGAGTGAAGCTGGA | |
| | CAGCAGAGAGAGGCCCTACGCCTGTCCTGTGGAAAGCTGC | |
| | GACAGACGGTTCAGCAGAAGCGACGAGCTGACCCGGCACA | |
| | TCAGAATCCACACCGGCCAGAAACCTTTCCAGTGCCGGAT | |
| | CTGCATGCGGAACTTCAGCAGATCCGACCACCTGACCACA | |
| | CACATCAGGACACACACAGGCGGAGGCAGACGGCGGAAG | |
| | AAGAGAACCAGCATCGAGACAAACATCCGCGTGGCCCTGG | |
| | AAAAGAGCTTCCTGGAAAACCAGAAGCCTACCAGCGAAGA | |
| | GATCACCATGATCGCCGACCAGCTGAACATGGAAAAAGAA | |
| | GTGATCCGCGTCTGGTTCTGCAACCGGCGGCAGAAAGAGA | |
| | AGCGGATCAACGGCAGCCGGAAGAGATGTGCTGCTGGTGT | |
| | TGGCGGAGGACCTGCTGGATGTCCTGCTCCTGGAAGCACC | |
| | CCTCTGAAGAAGCCTAGAAGAGGCAGCCTGTCTGAGGCCC | |
| | TGCTGCAGCTCCAGTTCGACGATGAAGATCTGGGAGCCCT | |
| | GCTGGGCAACAGCACAGATCCTGCCGTGTTTACCGATCTGG | |
| | CCAGCGTGGACAACAGCGAGTTTCAGCAGCTCCTGAACCA | |
| | GGGCATCCCTGTGGCTCCTCACACCACAGAGCCCATGCTGA | |
| | TGGAATACCCCGAGGCCATCACCAGACTGGTCACCGGTGC | |
| | TCAAAGACCTCCAGATCCAGCTCCTGCTCCACTGGGAGCAC | |
| | CTGGACTGCCTAATGGACTGCTGTCTGGCGACGAGGACTTC | |
| | AGCTCTATCGCCGACATGGATTTCAGCGCCCTGCTGAGCCA | |
| | GATCAGCTCTGGAGGTAGTGGTGGCGGATCCGGAATGGTG | |
| | GGCTCTCTGAATTGCATTGTGGCCGTGTCTCAGAACATGGG | |
| | CATCGGCAAGAACGGCGACCTTCCTTGGCCTCCTCTGCGGA | |
| | ACGAGCTGCGGTACTTCCACAGAATGACCACCACCAGCAG | |
| | CGTGGAAGGCAAGCAGAACCTGGTCATCATGGGCAAGAAA | |
| | ACCTGGTTCAGCATCGGCAGACCCCTGAAGGGCAGAATCA | |
| | ACCTGGTGCTGAGCAGAGAGCTGAAAGAGCCTCCTCAGGG | |
| | CGCCCACTTTCTGAGCAGATCTCTGGACGATGCCCTGAAGC | |
| | TGACCGAGCAGCCTGAACTGGCTAACAAGGTGGACATGGT | |
| | CTGGATCGTCGGCGGCAGCTCCGTGATCAAAGAAGCCATG | |
| | AATCACCCCGGCCACCTGAAACTGTTCGTGACCAGAATCAT | |
| | GCAGGACTTCGAGTCCGACACATTCTTCCCAGAGATCGACC | |
| | TGGAAAAGTACAAGCTGCTGCCAGAGTACCCCGGCGTGCT | |
| | GTCCGATGTGCAAGAGGAAAAGGGCATCAAGTACAAGTTC | |
| | GAGGTGTACGAGAAGAACGATTAAGCTAGCGAACAGAGA | |
| | AACAGGAGAATATGGGCCAAACAGGATATCTGTGGTAAGC | |
| | AGTTCCTGCCCCGGCTCAGGGCCAAGAACAGTTGGAACAG | |
| | CAGAATATGGGCCAAACAGGATATCTGTGGTAAGCAGTTC | |
| | CTGCCCCGGCTCAGGGCCAAGAACAGATGGTCCCCAGATG | |
| | CGGTCCCGCCCTCAGCAGTTTCTAGAGAACCATCAGATGTT | |
| | TCCAGGGTGCCCCAAGGACCTGAAATGACCCTGTGCCTTAT | |
| | TTGAACTAACCAATCAGTTCGCTTCTCGCTTCTGTTCGCGC | |
| | GCTTCTGCTCCCCGAGCTCTATATAAGCAGAGCTCGTTTAG | |
| | TGAACCGTCAGATCGCCACCACTAGTGCCACCATGGCTCTG | |
| | CCTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |
| TXN-124 | TAATGATGGGCGCTCGAGTAATGATGGGCGGTCGACTAAT | 49 |
| (12xZHFD1-YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| Lck-EFS-Nls-Zfhd1- | AGTAATGATGGGCGGTCGACTAATGATGGGCGCTCGAGTA | |
| ierNLS-p65- | ATGATGGGCGTCTAGAATCTAGAGGGTATATAATGGGGGC | |
| GGSGGGSG- | CAACCGGTCCTGCAGCGGAGGATCCGCCACCATGTTGAGC | |
| hDHFR(134)-MND- | AAGGGCGAGGAGGACAACATGGCCATCATCAAGGAGTTCA | |
| CD19CAR) | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGGCCATTAACTACGGGACATTC | |
| | TAGTGAAGCCACAGATGTAGAATGTCCCGTAGTTAATGGC | |
| | TGCCTACTGCCTCGGACTTCAAGGGACGCGTTGGCTTACCT | |
| | CCCATTGACCTTATGTACTGGGCAAAACCCATTGGAAAGTC | |
| | CCTATTGACTCAATGTACTTGGCTCCAATGGGACTTTCCTG | |
| | TTGACTCACCCCCTATTGACCTTATGTACTGGGCAAAACCC | |
| | AATGGAAAGTCCCTATTGAGTCAGTGTACTTGGCTCCAAAG | |
| | GGTTTTTCCCATTGACTAGTCGAGGAAGAGCATGCGTGAG | |
| | GCTCCGGTGCCCGTCAGTGGGCAGAGCGCACATCGCCCAC | |
| | AGTCCCCGAGAAGTTGGGGGGAGGGGTCGGCAATTGAACC | |
| | GGTGCCTAGAGAAGGTGGCGCGGGGTAAACTGGGAAAGTG | |
| | ATGTCGTGTACTGGCTCCGCCTTTTTCCCGAGGGTGGGGGA | |
| | GAACCGTATATAAGTGCAGTAGTCGCCGTGAACGTTCTTTT | |
| | TCGCAACGGGTTTGCCGCCAGAACACAGGCTAGACTAGTG | |
| | CCACCATGGATTATCCTGCCGCCAAGAGAGTGAAGCTGGA | |
| | CAGCAGAGAGAGGCCCTACGCCTGTCCTGTGGAAAGCTGC | |
| | GACAGACGGTTCAGCAGAAGCGACGAGCTGACCCGGCACA | |
| | TCAGAATCCACACCGGCCAGAAACCTTTCCAGTGCCGGAT | |
| | CTGCATGCGGAACTTCAGCAGATCCGACCACCTGACCACA | |
| | CACATCAGGACACACACAGGCGGAGGCAGACGGCGGAAG | |
| | AAGAGAACCAGCATCGAGACAAACATCCGCGTGGCCCTGG | |
| | AAAAGAGCTTCCTGGAAAACCAGAAGCCTACCAGCGAAGA | |
| | GATCACCATGATCGCCGACCAGCTGAACATGGAAAAAGAA | |
| | GTGATCCGCGTCTGGTTCTGCAACCGGCGGCAGAAAGAGA | |
| | AGCGGATCAACGGCAGCCGGAAGAGATGTGCTGCTGGTGT | |
| | TGGCGGAGGACCTGCTGGATGTCCTGCTCCTGGAAGCACC | |
| | CCTCTGAAGAAGCCTAGAAGAGGCAGCCTGTCTGAGGCCC | |
| | TGCTGCAGCTCCAGTTCGACGATGAAGATCTGGGAGCCCT | |
| | GCTGGGCAACAGCACAGATCCTGCCGTGTTTACCGATCTGG | |
| | CCAGCGTGGACAACAGCGAGTTTCAGCAGCTCCTGAACCA | |
| | GGGCATCCCTGTGGCTCCTCACACCACAGAGCCCATGCTGA | |
| | TGGAATACCCCGAGGCCATCACCAGACTGGTCACCGGTGC | |
| | TCAAAGACCTCCAGATCCAGCTCCTGCTCCACTGGGAGCAC | |
| | CTGGACTGCCTAATGGACTGCTGTCTGGCGACGAGGACTTC | |
| | AGCTCTATCGCCGACATGGATTTCAGCGCCCTGCTGAGCCA | |
| | GATCAGCTCTGGAGGTAGTGGTGGCGGATCCGGAATGGTG | |
| | GGCTCTCTGAATTGCATTGTGGCCGTGTCTCAGAACATGGG | |
| | CATCGGCAAGAACGGCGACCTTCCTTGGCCTCCTCTGCGGA | |
| | ACGAGCTGCGGTACTTCCACAGAATGACCACCACCAGCAG | |
| | CGTGGAAGGCAAGCAGAACCTGGTCATCATGGGCAAGAAA | |
| | ACCTGGTTCAGCATCGGCAGACCCCTGAAGGGCAGAATCA | |
| | ACCTGGTGCTGAGCAGAGAGCTGAAAGAGCCTCCTCAGGG | |
| | CGCCCACTTTCTGAGCAGATCTCTGGACGATGCCCTGAAGC | |
| | TGACCGAGCAGCCTGAACTGGCTAACAAGGTGGACATGGT | |
| | CTGGATCGTCGGCGGCAGCTCCGTGATCAAAGAAGCCATG | |
| | AATCACCCCGGCCACCTGAAACTGTTCGTGACCAGAATCAT | |
| | GCAGGACTTCGAGTCCGACACATTCTTCCCAGAGATCGACC | |
| | TGGAAAAGTACAAGCTGCTGCCAGAGTACCCCGGCGTGCT | |
| | GTCCGATGTGCAAGAGGAAAAGGGCATCAAGTACAAGTTC | |
| | GAGGTGTACGAGAAGAACGATTAAGCTAGCGAACAGAGA | |
| | AACAGGAGAATATGGGCCAAACAGGATATCTGTGGTAAGC | |
| | AGTTCCTGCCCCGGCTCAGGGCCAAGAACAGTTGGAACAG | |
| | CAGAATATGGGCCAAACAGGATATCTGTGGTAAGCAGTTC | |
| | CTGCCCCGGCTCAGGGCCAAGAACAGATGGTCCCCAGATG | |
| | CGGTCCCGCCCTCAGCAGTTTCTAGAGAACCATCAGATGTT | |
| | TCCAGGGTGCCCCAAGGACCTGAAATGACCCTGTGCCTTAT | |
| | TTGAACTAACCAATCAGTTCGCTTCTCGCTTCTGTTCGCGC | |
| | GCTTCTGCTCCCCGAGCTCTATATAAGCAGAGCTCGTTTAG | |
| | TGAACCGTCAGATCGCCACCACTAGTGCCACCATGGCTCTG | |
| | CCTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |
| TXN-125 | TAATGATGGGCGCTCGAGTAATGATGGGCGGTCGACTAAT | 50 |
| (12xZHFD1-YB- | GATGGGCGCTCGAGTAATGATGGGCGTCTAGCTAATGATG | |
| TATA-mCherry- | GGCGCTCGAGTAATGATGGGCGGTCGACTAATGATGGGCG | |
| shRNA targeting | CTCGAGTAATGATGGGCGTCTAGCTAATGATGGGCGCTCG | |
| Lck-EFS-Nls-Zfhd1- | AGTAATGATGGGCGGTCGACTAATGATGGGCGCTCGAGTA | |
| ierNLS-p65- | ATGATGGGCGTCTAGAATCTAGAGGGTATATAATGGGGGC | |
| GGSGGGSG- | CAACCGGTCCTGCAGCGGAGGATCCGCCACCATGTTGAGC | |
| hDHFR(134)-MND- | AAGGGCGAGGAGGACAACATGGCCATCATCAAGGAGTTCA | |
| CD19CAR) | TGCGCTTCAAGGTGCACATGGAGGGCTCCGTGAACGGCCA | |
| | CGAGTTCGAGATCGAGGGCGAGGGCGAGGGCCGCCCCTAC | |
| | GAGGGCACCCAGACCGCCAAGCTGAAGGTGACCAAGGGC | |
| | GGCCCCCTGCCCTTCGCCTGGGACATCCTGTCCCCTCAGTT | |
| | CATGTACGGCTCCAAGGCCTACGTGAAGCACCCCGCCGAC | |
| | ATCCCCGACTACTTGAAGCTGTCCTTCCCCGAGGGCTTCAA | |
| | GTGGGAGCGCGTGATGAACTTCGAGGACGGCGGCGTGGTG | |
| | ACCGTGACCCAGGACTCCTCCCTGCAGGACGGCGAGTTCA | |
| | TCTACAAGGTGAAGCTGCGCGGCACCAACTTCCCCTCCGAC | |
| | GGCCCCGTAATGCAGAAGAAGACCATGGGCTGGGAGGCCT | |
| | CCTCCGAGCGGATGTACCCCGAGGACGGCGCCCTGAAGGG | |
| | CGAGATCAAGCAGAGGCTGAAGCTGAAGGACGGCGGCCA | |
| | CTACGACGCCGAGGTCAAGACCACCTACAAGGCCAAGAAG | |
| | CCCGTGCAGCTGCCCGGCGCCTACAACGTCAACATCAAGC | |
| | TGGACATCACCTCCCACAACGAGGACTACACCATCGTGGA | |
| | ACAGTACGAGCGCGCCGAGGGCCGCCACTCCACCGGCGGC | |
| | ATGGACGAGCTGTACAAGTAATAGGAATTCAAGAAGGTAT | |
| | ATTGCTGTTGACAGTGAGCGTTCATTGAAGAGCGGAATTAT | |
| | TAGTGAAGCCACAGATGTAATAATTCCGCTCTTCAATGAAT | |
| | GCCTACTGCCTCGGACTTCAAGGGACGCGTTGGCTTACCTC | |
| | CCATTGACCTTATGTACTGGGCAAAACCCATTGGAAAGTCC | |
| | CTATTGACTCAATGTACTTGGCTCCAATGGGACTTTCCTGT | |
| | TGACTCACCCCCTATTGACCTTATGTACTGGGCAAAACCCA | |
| | ATGGAAAGTCCCTATTGAGTCAGTGTACTTGGCTCCAAAGG | |
| | GTTTTTCCCATTGACTAGTCGAGGAAGAGCATGCGTGAGGC | |
| | TCCGGTGCCCGTCAGTGGGCAGAGCGCACATCGCCCACAG | |
| | TCCCCGAGAAGTTGGGGGGAGGGGTCGGCAATTGAACCGG | |
| | TGCCTAGAGAAGGTGGCGCGGGGTAAACTGGGAAAGTGAT | |
| | GTCGTGTACTGGCTCCGCCTTTTTCCCGAGGGTGGGGGAGA | |
| | ACCGTATATAAGTGCAGTAGTCGCCGTGAACGTTCTTTTTC | |
| | GCAACGGGTTTGCCGCCAGAACACAGGCTAGACTAGTGCC | |
| | ACCATGGATTATCCTGCCGCCAAGAGAGTGAAGCTGGACA | |
| | GCAGAGAGAGGCCCTACGCCTGTCCTGTGGAAAGCTGCGA | |
| | CAGACGGTTCAGCAGAAGCGACGAGCTGACCCGGCACATC | |
| | AGAATCCACACCGGCCAGAAACCTTTCCAGTGCCGGATCT | |
| | GCATGCGGAACTTCAGCAGATCCGACCACCTGACCACACA | |
| | CATCAGGACACACACAGGCGGAGGCAGACGGCGGAAGAA | |
| | GAGAACCAGCATCGAGACAAACATCCGCGTGGCCCTGGAA | |
| | AAGAGCTTCCTGGAAAACCAGAAGCCTACCAGCGAAGAGA | |
| | TCACCATGATCGCCGACCAGCTGAACATGGAAAAAGAAGT | |
| | GATCCGCGTCTGGTTCTGCAACCGGCGGCAGAAAGAGAAG | |
| | CGGATCAACGGCAGCCGGAAGAGATGTGCTGCTGGTGTTG | |
| | GCGGAGGACCTGCTGGATGTCCTGCTCCTGGAAGCACCCCT | |
| | CTGAAGAAGCCTAGAAGAGGCAGCCTGTCTGAGGCCCTGC | |
| | TGCAGCTCCAGTTCGACGATGAAGATCTGGGAGCCCTGCT | |
| | GGGCAACAGCACAGATCCTGCCGTGTTTACCGATCTGGCC | |
| | AGCGTGGACAACAGCGAGTTTCAGCAGCTCCTGAACCAGG | |
| | GCATCCCTGTGGCTCCTCACACCACAGAGCCCATGCTGATG | |
| | GAATACCCCGAGGCCATCACCAGACTGGTCACCGGTGCTC | |
| | AAAGACCTCCAGATCCAGCTCCTGCTCCACTGGGAGCACCT | |
| | GGACTGCCTAATGGACTGCTGTCTGGCGACGAGGACTTCA | |
| | GCTCTATCGCCGACATGGATTTCAGCGCCCTGCTGAGCCAG | |
| | ATCAGCTCTGGAGGTAGTGGTGGCGGATCCGGAATGGTGG | |
| | GCTCTCTGAATTGCATTGTGGCCGTGTCTCAGAACATGGGC | |
| | ATCGGCAAGAACGGCGACCTTCCTTGGCCTCCTCTGCGGAA | |
| | CGAGCTGCGGTACTTCCACAGAATGACCACCACCAGCAGC | |
| | GTGGAAGGCAAGCAGAACCTGGTCATCATGGGCAAGAAAA | |
| | CCTGGTTCAGCATCGGCAGACCCCTGAAGGGCAGAATCAA | |
| | CCTGGTGCTGAGCAGAGAGCTGAAAGAGCCTCCTCAGGGC | |
| | GCCCACTTTCTGAGCAGATCTCTGGACGATGCCCTGAAGCT | |
| | GACCGAGCAGCCTGAACTGGCTAACAAGGTGGACATGGTC | |
| | TGGATCGTCGGCGGCAGCTCCGTGATCAAAGAAGCCATGA | |
| | ATCACCCCGGCCACCTGAAACTGTTCGTGACCAGAATCATG | |
| | CAGGACTTCGAGTCCGACACATTCTTCCCAGAGATCGACCT | |
| | GGAAAAGTACAAGCTGCTGCCAGAGTACCCCGGCGTGCTG | |
| | TCCGATGTGCAAGAGGAAAAGGGCATCAAGTACAAGTTCG | |
| | AGGTGTACGAGAAGAACGATTAAGCTAGCGAACAGAGAA | |
| | ACAGGAGAATATGGGCCAAACAGGATATCTGTGGTAAGCA | |
| | GTTCCTGCCCCGGCTCAGGGCCAAGAACAGTTGGAACAGC | |
| | AGAATATGGGCCAAACAGGATATCTGTGGTAAGCAGTTCC | |
| | TGCCCCGGCTCAGGGCCAAGAACAGATGGTCCCCAGATGC | |
| | GGTCCCGCCCTCAGCAGTTTCTAGAGAACCATCAGATGTTT | |
| | CCAGGGTGCCCCAAGGACCTGAAATGACCCTGTGCCTTATT | |
| | TGAACTAACCAATCAGTTCGCTTCTCGCTTCTGTTCGCGCG | |
| | CTTCTGCTCCCCGAGCTCTATATAAGCAGAGCTCGTTTAGT | |
| | GAACCGTCAGATCGCCACCACTAGTGCCACCATGGCTCTGC | |
| | CTGTGACAGCTCTGCTGCTGCCTCTGGCTCTGCTTCTGCAT | |
| | GCCGCCAGACCTGACATCCAGATGACCCAGACAACCAGCA | |
| | GCCTGTCTGCCAGCCTGGGCGATAGAGTGACCATCAGCTGT | |
| | AGAGCCAGCCAGGACATCAGCAAGTACCTGAACTGGTATC | |
| | AGCAGAAACCCGACGGCACCGTGAAGCTGCTGATCTACCA | |
| | CACCAGCAGACTGCACAGCGGCGTGCCAAGCAGATTTTCT | |
| | GGCAGCGGCTCTGGCACCGACTACAGCCTGACAATCAGCA | |
| | ACCTGGAACAAGAGGATATCGCTACCTACTTCTGCCAGCA | |
| | AGGCAACACCCTGCCTTACACCTTTGGCGGAGGCACCAAG | |
| | CTGGAAATCACAGGCGGCGGAGGAAGCGGAGGCGGAGGA | |
| | TCTGGTGGTGGTGGATCTGAAGTGAAACTGCAAGAGTCTG | |
| | GCCCTGGCCTGGTGGCCCCATCTCAATCTCTGAGCGTGACC | |
| | TGTACCGTCAGCGGAGTGTCCCTGCCTGATTATGGCGTGTC | |
| | CTGGATTCGGCAGCCTCCTAGAAAAGGCCTGGAATGGCTG | |
| | GGCGTGATCTGGGGCAGCGAGACAACCTACTACAACAGCG | |
| | CCCTGAAGTCCCGGCTGACCATCATCAAGGACAACTCCAA | |
| | GAGCCAGGTGTTCCTGAAGATGAACAGCCTGCAGACCGAC | |
| | GACACCGCCATCTACTATTGCGCCAAGCACTACTACTACGG | |
| | CGGCAGCTACGCCATGGATTATTGGGGCCAGGGCACCAGC | |
| | GTGACCGTGTCTAGTACAACAACCCCTGCTCCTCGGCCTCC | |
| | TACACCAGCTCCTACAATTGCCAGCCAGCCACTGTCTCTGA | |
| | GGCCCGAAGCTTGTAGACCTGCTGCTGGCGGAGCCGTGCA | |
| | TACAAGAGGACTGGATTTCGCCTGCGACATCTACATCTGGG | |
| | CCCCTCTGGCTGGAACATGTGGCGTGCTGCTGCTGAGCCTG | |
| | GTCATCACCCTGTATTGCAAGCGGGGCAGAAAGAAACTGC | |
| | TCTACATCTTCAAGCAGCCCTTCATGCGGCCCGTGCAGACC | |
| | ACACAAGAGGAAGATGGCTGCTCCTGCAGATTCCCCGAGG | |
| | AAGAAGAAGGCGGCTGCGAGCTGAGAGTGAAGTTCAGCA | |
| | GATCCGCCGACGCTCCCGCCTATAAGCAGGGACAGAACCA | |
| | GCTGTACAACGAGCTGAACCTGGGGAGAAGAGAAGAGTAC | |
| | GACGTGCTGGACAAGCGGAGAGGCAGAGATCCTGAAATGG | |
| | GCGGCAAGCCCAGACGGAAGAATCCTCAAGAGGGCCTGTA | |
| | TAATGAGCTGCAGAAAGACAAGATGGCCGAGGCCTACAGC | |
| | GAGATCGGAATGAAGGGCGAGCGCAGAAGAGGCAAGGGA | |
| | CACGATGGACTGTACCAGGGCCTGAGCACCGCCACCAAGG | |
| | ATACCTATGATGCCCTGCACATGCAGGCCCTGCCTCCAAGA | |
| | TAA | |

What is claimed is:

1. A set of nucleic acid sequences comprising:
a first nucleic acid sequence that encodes a transcription factor activation domain;
a second nucleic acid sequence that encodes a transcription factor DNA binding domain that binds to a specific nucleotide binding site;
a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and
a fourth nucleic acid sequence that encodes an inhibitory RNA molecule, the fourth nucleic acid sequence being operably linked to an inducible promoter comprising the specific nucleotide binding site;
wherein the transcription factor activation domain and the transcription factor DNA binding domain interact to form a transcription factor capable of activating transcription of the inhibitory RNA molecule upon binding to the specific nucleotide binding site.

2. The set of nucleic acid sequences of claim 1, wherein the first, second, third, and fourth nucleic acid sequences are each carried in a single vector, or wherein the first, second, third, and fourth nucleic acid sequences are each carried in separate vectors.

3. The set of nucleic acid sequences of claim 1, wherein the first, second, and third nucleic acid sequences are each carried in a single vector.

4. The set of nucleic acid sequences of claim 1, wherein the first and third nucleic acid sequences are each carried in a single vector.

5. The set of nucleic acid sequences of claim 1, wherein the second and third nucleic acid sequences are each carried in a single vector.

6. The set of nucleic acid sequences of claim 5, wherein the vector comprising the second and third nucleic acid sequences comprises a nucleic acid sequence of SEQ ID NO: 21.

7. The set of nucleic acid sequences of claim 1, wherein the vector is a viral vector or a non-viral vector.

8. The set of nucleic acid sequences of claim 7, wherein the viral vector is derived from adenovirus, adeno-associated virus (AAV), alphavirus, flavivirus, herpes virus, measles virus, rhabdovirus, retrovirus, lentivirus, Newcastle disease virus (NDV), poxvirus, and picornavirus.

9. The set of nucleic acid sequences of claim 1, wherein the transcription factor activation domain is selected from the group consisting of p65, VP16, NFAT1, VP64, p300, synergistic activation mediator (SAM), VPR.

10. The set of nucleic acid sequences of claim 9, wherein the transcription factor activation domain is p65.

11. The set of nucleic acid sequences of claim 1, wherein the transcription factor DNA binding domain is a zinc finger protein.

12. The set of nucleic acid sequences of claim 1, wherein the transcription factor DNA binding domain is a transcription activator-like (TAL) protein.

13. The set of nucleic acid sequences of any one of claim 1, wherein the transcription factor DNA binding domain is selected from the group consisting of zinc finger homeodomain 1 (ZFHD1), Cas9, Cas12.

14. The set of nucleic acid sequences of claim 13, wherein the transcription factor DNA binding domain is ZFHD1.

15. The set of nucleic acid sequences of claim 1, wherein the DRD is selected from the group consisting of human carbonic anhydrase 2 (CA2; SEQ ID NO: 30), FK506-binding protein (FKBP; SEQ ID NO: 31), E. coli DHFR (ecDHFR; SEQ ID NO: 32), human DHFR (hDHFR; SEQ ID NO: 33), human estrogen receptor (ER; SEQ ID NO: 34), phosphodiesterase 5 (PDE5; SEQ ID NO: 35), and PDE5 ligand binding domain (SEQ ID NO: 36) and variants thereof, wherein the variants of CA2, hDHFR, ecDHFR, ER, FKBP, PDE5, and PDE5 ligand binding domain have at least 85% amino acid sequence identity to SEQ ID NOs: 30-36.

16. The set of nucleic acid sequences of claim 15, wherein the DRD is hDHFR.

17. The set of nucleic acid sequences of claim 15, wherein the DRD is CA2.

18. The set of nucleic acid sequences of claim 1, wherein the DRD is responsive to a ligand selected from the group comprising: Acetazolamide (ACZ), Methotrexate (MTX), and Trimethoprim (TMP).

19. The set of nucleic acid sequences of claim 1, wherein the inhibitory RNA molecule is an shRNA molecule derived from a micro RNA (miRNA)-based scaffold.

20. The set of nucleic acid sequences of claim 1, wherein the inhibitory RNA molecule comprises one of SEQ ID NOs: 5-17 or SEQ ID NOs: 41-43.

21. The set of nucleic acid sequences of claim 1, wherein the inducible promoter comprises an RNA polymerase II promoter.

22. The set of nucleic acid sequences of claim 21, wherein the RNA polymerase II promoter is derived from ubiquitin C (UbiC), phosphoglycerate kinase (PGK), cytomegalovirus (CMV), beta-actin with an upstream CMV IV enhancer (CAGGS), or elongation factor 1 alpha (EF1A).

23. The set of nucleic acid sequences of claim 22, wherein the RNA polymerase II promoter is derived from ubiquitin C.

24. The set of nucleic acid sequences of claim 1 wherein the inducible promoter comprises a nucleic acid sequence of one of SEQ ID NOs: 1-3.

25. The set of nucleic acid sequences of claim 1, wherein the fourth nucleic acid sequence comprises a nucleic acid sequence of SEQ ID NO: 5-17 or 41-43.

26. An engineered cell comprising the set of nucleic acid sequences of claim 1.

27. The engineered cell of claim 26, wherein the cell is an immune cell.

28. The engineered cell of claim 27, wherein the immune cell is a T cell, a natural killer cell, or a tumor infiltrating lymphocyte.

29. The engineered cell of claim 26, wherein the cell is a cancer cell.

30. A pharmaceutical composition comprising the engineered cell of claim 25 and a pharmaceutically acceptable carrier.

31. A method of producing an engineered cell comprising introducing into the cell the set of nucleic acid sequences of claim 1.

32. The method of producing an engineered cell of claim 31, wherein the cell is an immune cell.

33. The method of producing an engineered cell of claim 32, wherein the immune cell is a T cell, a natural killer (NK) cell, or a tumor infiltrating lymphocyte (TIL).

34. The method of producing an engineered cell of claim 31, wherein the cell is a cancer cell.

35. The method of producing an engineered cell of claim 31, wherein the first, second, and fourth nucleic acid sequences are located on a single vector.

36. The method of producing an engineered cell of claim 31, wherein the first, second, and fourth nucleic acid sequences are located on separate vectors.

37. The method of producing an engineered cell of claim 31, wherein the first and third nucleic acid sequences are located on a single vector.

38. The method of producing an engineered cell of of claim 31, wherein the second and third nucleic acid sequences are located on a single vector.

39. The method of producing an engineered cell of claim 38, wherein the vector comprising the second and third nucleic acid sequences comprises a nucleic acid sequence of SEQ ID NO: 21.

40. The method of producing an engineered cell of claim 35, wherein the vector is a viral vector or a non-viral vector.

41. The method of producing an engineered cell of claim 40, wherein the viral vector is derived from adenovirus, adeno-associated virus (AAV), alphavirus, flavivirus, herpes virus, measles virus, rhabdovirus, retrovirus, lentivirus, Newcastle disease virus (NDV), poxvirus, and picornavirus.

42. A method of delivering a regulatable inhibitory RNA molecule to a subject comprising administering to the subject the set of nucleic acid sequences of claim 1, the engineered cell of claim 25, or the pharmaceutical composition of claim 30.

43. A method for treating a disease in a subject in need thereof comprising administering to the subject the set of nucleic acid sequences of claim 1, the engineered cell of claim 25, or the pharmaceutical composition of claim 30.

44. The method for treating a disease in a subject in need thereof of claim 43, wherein the disease is a neurodegenerative disease.

45. The method for treating a disease in a subject in need thereof of claim 44, wherein the neurodegenerative disease is Alzheimer's disease or Huntington's disease.

46. The method for treating a disease in a subject in need thereof of claim 43, wherein the disease is osteoarthritis.

47. The method for treating a disease in a subject in need thereof of claim 43, wherein the disease is age-related macular degeneration.

48. A method for regulation of an immune cell comprising administering to the immune cell the set of nucleic acid sequences of claim 1.

49. The method of claim 48, wherein the immune cell is a T cell, a natural killer (NK) cell, or a tumor infiltrating lymphocyte (TIL).

50. The method of claim 49, wherein the immune cell is a T cell.

51. The method of claim 48, wherein the inhibitory RNA molecule downregulates transcription of a T cell receptor signaling gene.

52. The method of claim 51, wherein the T cell receptor signaling gene is selected from the group consisting of Lck, zeta-chain-associated protein kinase-70 (ZAP70), unc119, or linker for activation of T cells (LAT).

53. The method of claim 48, wherein the immune cell expresses a chimeric antigen receptor.

54. A kit comprising the set of nucleic acid sequences of claim 1, the engineered cell of claim 25, or the pharmaceutical composition of claim 30, and a ligand, wherein the DRD is responsive to the ligand.

55. The kit of claim 54, wherein the ligand is selected from the group comprising: Acetazolamide (ACZ), Methotrexate (MTX), and Trimethoprim (TMP).


SEQUENCE LISTING (50 sequences)

SEQ ID NO: 1 - DNA, 400 bp, Homo sapiens
  source 1..400: mol_type = genomic DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggt

SEQ ID NO: 2 - DNA, 338 bp, Homo sapiens
  source 1..338: mol_type = genomic DNA
gatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagct

SEQ ID NO: 3 - DNA, 1319 bp, synthetic construct
  source 1..1319: mol_type = other DNA
cggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgtttgctgttgacagtgagcgatcaagctgaccctgaagttcattagtgaagccacagatgtaatgaacttcagggtcagcttgcctgcctactgcctcggacttcaagggggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttag

SEQ ID NO: 4 - DNA, 22 bp, synthetic construct
  source 1..22: mol_type = other DNA
tcaagctgaccctgaagttcat

SEQ ID NO: 5 - DNA, 21 bp, synthetic construct
  source 1..21: mol_type = other DNA
caggcgtagatcaccagaata

SEQ ID NO: 6 - DNA, 21 bp, synthetic construct
  source 1..21: mol_type = other DNA
cgcaacgtcctgctggttaac

SEQ ID NO: 7 - DNA, 21 bp, synthetic construct
  source 1..21: mol_type = other DNA
gaacttggctgcggcaacttt

SEQ ID NO: 8 - DNA, 21 bp, synthetic construct
  source 1..21: mol_type = other DNA
cctactctgtgtaatagaata

SEQ ID NO: 9 - DNA, 21 bp, synthetic construct
  source 1..21: mol_type = other DNA
gggctccagattacgagaatc

SEQ ID NO: 10 - DNA, 21 bp, synthetic construct
  source 1..21: mol_type = other DNA
gtccattgatgattacgtgaa

SEQ ID NO: 11 - DNA, 21 bp, synthetic construct
  source 1..21: mol_type = other DNA
gctgatggacgagaccatgaa

SEQ ID NO: 12 - DNA, 21 bp, synthetic construct
  source 1..21: mol_type = other DNA
gaaggagttgaaggcctacaa

SEQ ID NO: 13 - DNA, 21 bp, synthetic construct
  source 1..21: mol_type = other DNA
tgcccagcgacaatcactgaa

SEQ ID NO: 14 - DNA, 21 bp, synthetic construct
  source 1..21: mol_type = other DNA
gacaatcactgaacgccgaag

SEQ ID NO: 15 - DNA, 21 bp, synthetic construct
  source 1..21: mol_type = other DNA
tggttcagttacgggttaatt

SEQ ID NO: 16 - DNA, 21 bp, synthetic construct
  source 1..21: mol_type = other DNA
tgttgccgcagcatcactaat

SEQ ID NO: 17 - DNA, 21 bp, synthetic construct
  source 1..21: mol_type = other DNA
gcactcaagaaggacacaata

SEQ ID NO: 18 - DNA, 423 bp, synthetic construct
  source 1..423: mol_type = other DNA
gatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctcatatgggcaagctgaccctgaagttcatctgtcaagaggtctacttgaagtcccagtcgaacgggaattcgtttcaaaagtaga

SEQ ID NO: 19 - DNA, 1261 bp, synthetic construct
  source 1..1261: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtcgctagcgctaccggtcgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgtcaagctgaccctgaagttcattagtgaagccacagatgtaatgaacttcagggtcagcttgctgcctactgcctcggacttcaaggg

SEQ ID NO: 20 - DNA, 1319 bp, synthetic construct; identical to SEQ ID NO: 3
  source 1..1319: mol_type = other DNA

SEQ ID NO: 21 - DNA, 2283 bp, synthetic construct
  source 1..2283: mol_type = other DNA
atggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagactaggccaccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcaagtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctg

SEQ ID NO: 22 - DNA, 1121 bp, synthetic construct
  source 1..1121: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaaataggggttcaatataaaaagccggcagagagctgtccaagtccgctagcgctaccggtcgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgtcaagctgaccctgaagttcattagtgaagccacagatgtaatgaacttcagggtcagcttgctgcctactgcctcggacttcaaggg

SEQ ID NO: 23 - DNA, 1029 bp, synthetic construct
  source 1..1029: mol_type = other DNA
taatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagctctagagggtatataatgggggccacgctagcgctaccggtcgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcggacaatcactgaacgccgaagtagtgaagccacagatgtacttcggcgttcagtgattgtctgcctactgcctcggacttcaaggg

SEQ ID NO: 24 - DNA, 1119 bp, synthetic construct
  source 1..1119: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaaataggggttcaatataaaaagccggcagagagctgtccaagtccgctagcgctaccggtcgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgtggttcagttacgggttaatttagtgaagccacagatgtaaattaacccgtaactgaaccatgcctactgcctcggacttcaaggg

SEQ ID NO: 25 - DNA, 3859 bp, synthetic construct
  source 1..3859: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgtcaagctgaccctgaagttcattagtgaagccacagatgtaatgaacttcagggtcagcttgctgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagactaggccaccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcaagtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctg

SEQ ID NO: 26 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgcaggcgtagatcaccagaatatagtgaagccacagatgtatattctggtgatctacgcctgtgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 27 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgcgcaacgtcctgctggttaactagtgaagccacagatgtagttaaccagcaggacgttgcgtgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 28 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcggaacttggctgcggcaacttttagtgaagccacagatgtaaaagttgccgcagccaagttctgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 29 - DNA, 3023 bp, synthetic construct
  source 1..3023: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagactagtggccaccatgggttgtagactcctgtgctgtgcagttttgtgccttctcggggccgtgccaatagatactgaagtgacgcaaaccccaaagcacttggtaatgggaatgactaataaaaaaagcctgaagtgtgagcagcacatgggtcaccgagcaatgtattggtacaaacaaaaagcgaagaagccaccggagcttatgtttgtatattcttatgagaagttgtcaatcaatgagagcgtcccctcccggttctcaccggagtgtcctaacagtagcttgctcaatctccatctccatgctctccagccggaagacagcgctctctacctttgtgcaagttctcccacgagcggaatttatgaacagtactttgggcctggtacgcgcctcacggtcactgaggacttgaagaatgtcttccctccagaggttgccgtgtttgagccctcagaggctgaaatttctcacacccaaaaggccacactcgtctgtttggctacgggtttctatcccgatcacgtggaactttcttggtgggtcaatggaaaagaagttcacagtggtgtcagtacagaccctcagccacttaaggagcaaccagctctgaacgattctcgatactgcttgtcatctcgcctcagagtcagcgctactttctggcaaaacccaagaaaccatttcagatgccaagttcagttctatggcttgtccgagaacgatgagtggacccaagaccgcgcgaagccagttacacagatagtaagcgccgaagcgtggggtcgcgccgactgtggcttcacatctgagagttatcagcaaggagtactgagtgcaactattttgtatgagatattgctgggaaaagcgacgttgtacgccgtgctggtgtccgctctcgtgctcatggccatggtaaagcgaaaggatagtaggggagctacgaacttttcactcctgaagcaagcaggtgacgtggaggagaacccaggtccgatgttgttggaactgattcctctccttggtatccatttcgtcctccgaaccgcgcgggctcagtctgtcactcagccagatatacacataacagtttctgagggcgctagcttggagcttcggtgcaactatagttacggtgccacaccgtaccttttctggtacgtgcagagccccggacaggggcttcaactcctcctgaagtacttttcaggggacactttggttcagggaattaagggttttgaggctgaatttaagaggtcacagtccagcttcaacttgcgaaaaccctcagttcattggagtgatgccgcagagtatttctgtgcagtaggagcgccatctggcgctggctcatatcaattgacctttgggaagggaaccaaactgagcgttattcccaatatccaaaacccagacccagcggtttatcaattgagggattccaaatctagtgataaatcagtgtgcctttttactgactttgatagtcagacgaacgtatcccaaagcaaggatagtgacgtctacataacagataagacagttctcgatatgcgctcaatggattttaagtctaacagcgcggtggcatggagtaacaagtccgactttgcctgcgcgaatgctttcaataacagtattatacccgaagacacgttctttccttcacccgaaagttcttgtgacgtcaagctggtcgagaaaagtttcgaaactgatacgaacctgaatttccaaaatttgtccgttattggatttcgaatacttcttctgaaagtggcgggcttcaatttgttgatgacattgcgactttggtcatcctaa

SEQ ID NO: 30 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 31 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 32 - protein, 159 aa, Escherichia coli [sequence omitted]

SEQ ID NO: 33 - protein, 187 aa, Homo sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 34 - protein, 595 aa, Homo sapiens [sequence omitted]

SEQ ID NO: 35 - protein, 875 aa, Homo sapiens
MERAGPSFGQQRQQQQPQQQKQQQRDQDSVEAWLDDHWDFTFSYFVRKATREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 36 - protein, 327 aa, Homo sapiens
MEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 37 - DNA, 348 bp, Homo sapiens
  source 1..348: mol_type = genomic DNA
ctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctct

SEQ ID NO: 38 - DNA, 492 bp, synthetic construct
  source 1..492: mol_type = other DNA
gattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaaga

SEQ ID NO: 39 - DNA, 2050 bp, synthetic construct
  source 1..2050: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtg

SEQ ID NO: 40 - DNA, 12 bp, synthetic construct
  source 1..12: mol_type = other DNA
taatgatgggcg

SEQ ID NO: 41 - DNA, 21 bp, synthetic construct
  source 1..21: mol_type = other DNA
gcatgaactggtccgccatta

SEQ ID NO: 42 - DNA, 21 bp, synthetic construct
  source 1..21: mol_type = other DNA
gccattaactacgggacattc

SEQ ID NO: 43 - DNA, 21 bp, synthetic construct
  source 1..21: mol_type = other DNA
ttcattgaagagcggaattat

SEQ ID NO: 44 - DNA, 4836 bp, synthetic construct
  source 1..4836: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgtcaagctgaccctgaagttcattagtgaagccacagatgtaatgaacttcagggtcagcttgctgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 45 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgcctactctgtgtaatagaatatagtgaagccacagatgtatattctattacacagagtaggtgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 46 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcggggctccagattacgagaatctagtgaagccacagatgtagattctcgtaatctggagccctgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 47 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcggtccattgatgattacgtgaatagtgaagccacagatgtattcacgtaatcatcaatggactgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 48 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcggcatgaactggtccgccattatagtgaagccacagatgtataatggcggaccagttcatgctgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 49 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcggccattaactacgggacattctagtgaagccacagatgtagaatgtcccgtagttaatggctgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 50 - DNA, 4834 bp, synthetic construct
  source 1..4834: mol_type = other DNA
taatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaatctagagggtatataatgggggccaaccggtcctgcagcggaggatccgccaccatgttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaataggaattcaagaaggtatattgctgttgacagtgagcgttcattgaagagcggaattattagtgaagccacagatgtaataattccgctcttcaatgaatgcctactgcctcggacttcaagggacgcgttggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaaagggtttttcccattgactagtcgaggaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagactagtgccaccatggattatcctgccgccaagagagtgaagctggacagcagagagaggccctacgcctgtcctgtggaaagctgcgacagacggttcagcagaagcgacgagctgacccggcacatcagaatccacaccggccagaaacctttccagtgccggatctgcatgcggaacttcagcagatccgaccacctgaccacacacatcaggacacacacaggcggaggcagacggcggaagaagagaaccagcatcgagacaaacatccgcgtggccctggaaaagagcttcctggaaaaccagaagcctaccagcgaagagatcaccatgatcgccgaccagctgaacatggaaaaagaagtgatccgcgtctggttctgcaaccggcggcagaaagagaagcggatcaacggcagccggaagagatgtgctgctggtgttggcggaggacctgctggatgtcctgctcctggaagcacccctctgaagaagcctagaagaggcagcctgtctgaggccctgctgcagctccagttcgacgatgaagatctgggagccctgctgggcaacagcacagatcctgccgtgtttaccgatctggccagcgtggacaacagcgagtttcagcagctcctgaaccagggcatccctgtggctcctcacaccacagagcccatgctgatggaataccccgaggccatcaccagactggtcaccggtgctcaaagacctccagatccagctcctgctccactgggagcacctggactgcctaatggactgctgtctggcgacgaggacttcagctctatcgccgacatggatttcagcgccctgctgagccagatcagctctggaggtagtggtggcggatccggaatggtgggctctctgaattgcattgtggccgtgtctcagaacatgggcatcggcaagaacggcgaccttccttggcctcctctgcggaacgagctgcggtacttccacagaatgaccaccaccagcagcgtggaaggcaagcagaacctggtcatcatgggcaagaaaacctggttcagcatcggcagacccctgaagggcagaatcaacctggtgctgagcagagagctgaaagagcctcctcagggcgcccactttctgagcagatctctggacgatgccctgaagctgaccgagcagcctgaactggctaacaaggtggacatggtctggatcgtcggcggcagctccgtgatcaaagaagccatgaatcaccccggccacctgaaactgttcgtgaccagaatcatgcaggacttcgagtccgacacattcttcccagagatcgacctggaaaagtacaagctgctgccagagtaccccggcgtgctgtccgatgtgcaagaggaaaagggcatcaagtacaagttcgaggtgtacgagaagaacgattaagctagcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgccaccactagtgccaccatggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggattcggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa